SEQUENCE LISTING (106 sequences)

SEQ ID NO: 1 - protein, 17 aa, Homo sapiens
SHRLLAVCALVSLATAA

SEQ ID NO: 2 - protein, 22 aa, Homo sapiens
SHRLLAVCALVSLATAALLGHI

SEQ ID NO: 3 - protein, 16 aa, Artificial Sequence
  Synthetic Construct
AAALVSLATAALLGHI

SEQ ID NO: 4 - protein, 15 aa, Homo sapiens
KDILTLRLDVMMETE

SEQ ID NO: 5 - protein, 17 aa, Homo sapiens
AHGVFLLNSNAMDVVLQ

SEQ ID NO: 6 - protein, 22 aa, Homo sapiens
GAGLQLQKVTVLGVATAPQQVL

SEQ ID NO: 7 - protein, 15 aa, Homo sapiens
PRELSGSSPVLEETH

SEQ ID NO: 8 - protein, 20 aa, Homo sapiens
LGHILLHDFLLVPRELSGSS

SEQ ID NO: 9 - protein, 9 aa, Homo sapiens
SHRLLAVCA

SEQ ID NO: 10 - protein, 9 aa, Homo sapiens
HRLLAVCAL

SEQ ID NO: 11 - protein, 9 aa, Homo sapiens
RLLAVCALV

SEQ ID NO: 12 - protein, 9 aa, Homo sapiens
LLAVCALVS

SEQ ID NO: 13 - protein, 9 aa, Homo sapiens
LAVCALVSL

SEQ ID NO: 14 - protein, 9 aa, Homo sapiens
AVCALVSLA

SEQ ID NO: 15 - protein, 9 aa, Homo sapiens
VCALVSLAT

SEQ ID NO: 16 - protein, 9 aa, Homo sapiens
CALVSLATA

SEQ ID NO: 17 - protein, 9 aa, Homo sapiens
ALVSLATAA

SEQ ID NO: 18 - protein, 9 aa, Homo sapiens
LVSLATAAL

SEQ ID NO: 19 - protein, 9 aa, Homo sapiens
VSLATAALL

SEQ ID NO: 20 - protein, 9 aa, Homo sapiens
SLATAALLG

SEQ ID NO: 21 - protein, 9 aa, Homo sapiens
LATAALLGH

SEQ ID NO: 22 - protein, 9 aa, Homo sapiens
ATAALLGHI

SEQ ID NO: 23 - protein, 8 aa, Homo sapiens
KDILTLRD

SEQ ID NO: 24 - protein, 9 aa, Homo sapiens
DILTLRLDV

SEQ ID NO: 25 - protein, 9 aa, Homo sapiens
ILTLRLDVM

SEQ ID NO: 26 - protein, 9 aa, Homo sapiens
LTLRLDVMM

SEQ ID NO: 27 - protein, 9 aa, Homo sapiens
TLRLDVMME

SEQ ID NO: 28 - protein, 9 aa, Homo sapiens
LRLDVMMET

SEQ ID NO: 29 - protein, 9 aa, Homo sapiens
RLDVMMETE

SEQ ID NO: 30 - protein, 9 aa, Homo sapiens
AHGVFLLNS

SEQ ID NO: 31 - protein, 9 aa, Homo sapiens
HGVFLLNSN

SEQ ID NO: 32 - protein, 9 aa, Homo sapiens
GVFLLNSNA

SEQ ID NO: 33 - protein, 9 aa, Homo sapiens
VFLLNSNAM

SEQ ID NO: 34 - protein, 9 aa, Homo sapiens
FLLNSNAMD

SEQ ID NO: 35 - protein, 9 aa, Homo sapiens
LLSNNAMDV

SEQ ID NO: 36 - protein, 9 aa, Homo sapiens
LNSNAMDVV

SEQ ID NO: 37 - protein, 9 aa, Homo sapiens
NSNAMDVVL

SEQ ID NO: 38 - protein, 9 aa, Homo sapiens
SNAMDVVLQ

SEQ ID NO: 39 - protein, 9 aa, Homo sapiens
GAGLQLQKV

SEQ ID NO: 40 - protein, 9 aa, Homo sapiens
AGLQLQKVT

SEQ ID NO: 41 - protein, 9 aa, Homo sapiens
GLQLQKVTV

SEQ ID NO: 42 - protein, 9 aa, Homo sapiens
LQLQKVTVL

SEQ ID NO: 43 - protein, 9 aa, Homo sapiens
QLQKVTVLG

SEQ ID NO: 44 - protein, 9 aa, Homo sapiens
LQKVTVLGV

SEQ ID NO: 45 - protein, 9 aa, Homo sapiens
QKVTVLGVA

SEQ ID NO: 46 - protein, 9 aa, Homo sapiens
KVTVLGVAT

SEQ ID NO: 47 - protein, 9 aa, Homo sapiens
VTVLGVATA

SEQ ID NO: 48 - protein, 9 aa, Homo sapiens
TVLGVATAP

SEQ ID NO: 49 - protein, 9 aa, Homo sapiens
VLGVATAPQ

SEQ ID NO: 50 - protein, 9 aa, Homo sapiens
LGVATAPQQ

SEQ ID NO: 51 - protein, 9 aa, Homo sapiens
GVATAPQQV

SEQ ID NO: 52 - protein, 9 aa, Homo sapiens
VATAPQQVL

SEQ ID NO: 53 - protein, 9 aa, Artificial Sequence
  Synthetic Construct
AAALVSLAT

SEQ ID NO: 54 - protein, 9 aa, Artificial Sequence
  Synthetic Construct
AALVSLATA

SEQ ID NO: 55 - protein, 9 aa, Homo sapiens
PRELSGSSP

SEQ ID NO: 56 - protein, 9 aa, Homo sapiens
RELSGSSPV

SEQ ID NO: 57 - protein, 9 aa, Homo sapiens
ELSGSSPVL

SEQ ID NO: 58 - protein, 9 aa, Homo sapiens
LSGSSPVLE

SEQ ID NO: 59 - protein, 9 aa, Homo sapiens
SGSSPVLEE

SEQ ID NO: 60 - protein, 9 aa, Homo sapiens
GSSPVLEET

SEQ ID NO: 61 - protein, 9 aa, Homo sapiens
SSPVLEETH

SEQ ID NO: 62 - protein, 9 aa, Homo sapiens
LGHILLHDF

SEQ ID NO: 63 - protein, 9 aa, Homo sapiens
GHILLHDFL

SEQ ID NO: 64 - protein, 9 aa, Homo sapiens
HILLHDFLL

SEQ ID NO: 65 - protein, 9 aa, Homo sapiens
ILLHDFLLV

SEQ ID NO: 66 - protein, 10 aa, Homo sapiens
ILLHDFLLVP

SEQ ID NO: 67 - protein, 9 aa, Homo sapiens
LHDFLLVPR

SEQ ID NO: 68 - protein, 9 aa, Homo sapiens
HDFLLVPRE

SEQ ID NO: 69 - protein, 9 aa, Homo sapiens
DFLLVPREL

SEQ ID NO: 70 - protein, 9 aa, Homo sapiens
FLLVPRELS

SEQ ID NO: 71 - protein, 9 aa, Homo sapiens
LLVPRELSG

SEQ ID NO: 72 - protein, 9 aa, Homo sapiens
LVPRELSGS

SEQ ID NO: 73 - protein, 9 aa, Homo sapiens
VPRELSGSS

SEQ ID NO: 74 - protein, 15 aa, Homo sapiens
ILTIHFTGHSFIYGK

SEQ ID NO: 75 - protein, 18 aa, Homo sapiens
SYPSYKLENLSSSEMGYT

SEQ ID NO: 76 - protein, 19 aa, Homo sapiens
EMGYTATLTRTTPTFFPKD

SEQ ID NO: 77 - protein, 18 aa, Homo sapiens
ADQFLQLSTSLPSQYIGT

SEQ ID NO: 78 - protein, 17 aa, Homo sapiens
AMDVVLQPSPALSWRST

SEQ ID NO: 79 - protein, 20 aa, Homo sapiens
SHQFLSTHYNLHNLYGLTEA

SEQ ID NO: 80 - protein, 15 aa, Homo sapiens
TRPFVISRSTFAGHG

SEQ ID NO: 81 - protein, 19 aa, Homo sapiens
SVPEILQFNLLGVPLVGAD

SEQ ID NO: 82 - protein, 15 aa, Homo sapiens
VCGFLGNTSEELCVR

SEQ ID NO: 83 - protein, 22 aa, Homo sapiens
LGAFYPFMRNHNSLLSLPQEPY

SEQ ID NO: 84 - protein, 23 aa, Homo sapiens
QQAMRKALTLRYALLPHLYTLFH

SEQ ID NO: 85 - protein, 17 aa, Homo sapiens
LYTLFHQQAHVAGETVA

SEQ ID NO: 86 - protein, 25 aa, Homo sapiens
YTQVIFLARNNTIVNELVRVTSEGA

SEQ ID NO: 87 - protein, 18 aa, Homo sapiens
ATAPQQVLSNGVPVSNFT

SEQ ID NO: 88 - protein, 22 aa, Homo sapiens
GAFYPFMRNHNSLLSLPQEPYS

SEQ ID NO: 89 - protein, 17 aa, Homo sapiens
ADQFLQLSTSLPSQYIT

SEQ ID NO: 90 - protein, 19 aa, Homo sapiens
SVPEILQFNLLGVPLVGAD

SEQ ID NO: 91 - protein, 18 aa, Homo sapiens
EPAQQAMRKALTLRYALL

SEQ ID NO: 92 - protein, 18 aa, Homo sapiens
KKGRVLLNTTVAPLFFAD

SEQ ID NO: 93 - protein, 17 aa, Homo sapiens
GVIVRRQLDGRVLLNTT

SEQ ID NO: 94 - protein, 16 aa, Homo sapiens
LSPLMLSTSWTRITLW

SEQ ID NO: 95 - protein, 13 aa, Homo sapiens
VEALGSLPPPPAA

SEQ ID NO: 96 - protein, 20 aa, Homo sapiens
SHQFLSTHYNLHNLYGLTEA

SEQ ID NO: 97 - protein, 18 aa, Homo sapiens
NTIVNELVRVTSEGAGLQ

SEQ ID NO: 98 - protein, 14 aa, Homo sapiens
GDVWSSWEQLASSV

SEQ ID NO: 99 - protein, 15 aa, Homo sapiens
AAGFLGNTSEELCVR

SEQ ID NO: 100 - protein, 16 aa, Homo sapiens
VCALVSLATAALLGHI

SEQ ID NO: 101 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 102 - protein, 968 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWCAAALVSLATAALLGHI

SEQ ID NO: 103 - protein, 968 aa, Homo sapiens
AAALVSLATAALLGHIMGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 104 - protein, 952 aa, Homo sapiens
MGVRHPPCSHRLLAAAALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 105 - protein, 985 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWCAAALVSLATAALLGHIAHGVFLLNSNAMDVVLQ

SEQ ID NO: 106 - protein, 15 aa, Homo sapiens
VNELVRVTSEGAGLQ

(19) United States
(12) Patent Application Publication
(10) Pub. No.: US 2023/0321237 A1
(43) Pub. Date: Oct. 12, 2023

(54) REGULATORY T CELL EPITOPES

(71) Applicant: EPIVAX, INC., Providence, RI (US)

(72) Inventors: Anne De Groot, Providence, RI (US); William Martin, Providence, RI (US)

(21) Appl. No.: 18/040,226

(22) PCT Filed: Aug. 13, 2021
(86) PCT No.: PCT/US2021/045908

§ 371 Date: Feb. 1, 2023

Related U.S. Application Data
(60) Provisional application No. 63/065,029, filed on Aug. 13, 2020.

(51) Int. Cl. A61K 39/00; C07K 14/725; A61K 47/64; A61K 47/54; A61P 37/06; C12N 9/24; C12N 5/0783

(52) U.S. Cl. CPC A61K 39/4621; C07K 14/7051; A61K 39/4615; A61K 39/464839; A61K 47/643; A61K 47/545; A61P 37/06; C12N 9/2402; C12Y 302/0102; A61K 39/46434; C12N 5/0637; A61K 39/4611; C12N 2501/998; C07K 2319/33

(57) ABSTRACT

The present is directed to compositions comprising regulatory T cell epitopes, wherein said epitopes comprise a polypeptide comprising at least a portion of SEQ NOS: 1-73, fragments and/or variants thereof, as well as methods of producing and using the same.

### CROSS-REFERENCE TO RELATED APPLICATION

This application depends from and claims priority to U.S. Provisional Application No. 63/065,029 filed Aug. 13, 2020, the entire contents of which are incorporated herein by reference in their entirety.

### GOVERNMENT SUPPORT CLAUSE

The invention was made with government support under grant number TR002441 awarded by the National Institutes of Health (NIH). The government has certain rights in the invention.

### SEQUENCE LISTING

The instant application contains a Sequence Listing which has been submitted in ASCII format via EFS-Web and is hereby incorporated by reference in its entirety. Said ASCII copy, created on Mar. 26, 2020, is named EPIVAX GAA SEQS_ST25.txt and is 63 KB in size.

### FIELD

The present disclosure generally relates to a novel class of regulatory T cell epitopes (termed “Tregitopes”). The present disclosure provides Tregitope compounds and compositions (including one or more of e.g., polypeptides (which may be termed herein as “T_{reg} activating regulatory T-cell epitope”, “Tregitope”, or “T-cell epitope polypeptide”) having a sequence comprising, consisting of, or consisting essentially of one or more of SEQ ID NOS: 1-73 (in aspects, the polypeptides may be isolated, synthetic, or recombinant) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) as well as methods for their preparation and use.

### BACKGROUND

Artificial induction of tolerance to self or to foreign antigens is the goal of therapy for autoimmunity, transplantation allergy and other diseases. Immune response targeting autologous and non-autologous therapeutic proteins often limits clinical efficacy. Immune modulating treatments, inducing tolerance to therapeutic proteins compositions, may reduce the formation of anti-drug antibodies (ADA) improving clinical outcomes. Until recently, therapeutic tolerance induction relied on broad-based immune cell depleting therapies. These broad-based approaches weaken the immune system in general and leave many subjects vulnerable to opportunistic infections, autoimmune attack and cancer. There is a need in the art for less aggressive and more targeted approaches to the induction of immune tolerance.

Immune tolerance is regulated by a complex interplay between antigen presenting cells (APC), T cells, B cells, cytokines, chemokines, and surface receptors. Initial self/non-self discrimination occurs in the thymus during neonatal development where medullary epithelial cells express specific self protein epitopes to immature T cells. T cells recognizing self antigens with high affinity are deleted, but autoreactive T cells with moderate affinity sometimes avoid deletion and can be converted to so called ‘natural’ regulatory T cells (T_{Reg}) cells. These natural T_{Reg} cells are exported to the periphery and help to control latent autoimmune response.

A second form of tolerance develops in the periphery. In this case activated T cells are converted to an ‘adaptive’ T_{Reg} phenotype through the action of certain immune suppressive cytokines and chemokines such as IL-10, TGF-β and CCL19. The possible roles for these ‘adaptive’ T_{Reg} cells include dampening immune response following the successful clearance of an invading pathogen, controlling excessive inflammation caused by an allergic reaction, controlling excessive inflammation caused by low level or chronic infection, or possibly controlling inflammatory response targeting beneficial symbiotic bacteria.

Naturally occurring T_{Reg}s (including both natural T_{Reg}s and adaptive T_{Reg}s) are a critical component of immune regulation in the periphery. For example, upon activation of natural T_{Reg}s through their TCR, natural T_{Reg}s express immune modulating cytokines and chemokines. Activated natural T_{Reg}s may suppress nearby effector T cells through contact dependent and independent mechanisms. In addition, the cytokines released by these cells including, but not limited to, IL-10 and TGF-β, are capable of inducing antigen-specific adaptive T_{Reg}s. Despite extensive efforts, with few exceptions, the antigen specificity of natural T_{Reg}s, and more importantly natural T_{Reg}s circulating in clinically significant volumes, is still unknown.

There is need in the art for the identification of regulatory T cell epitopes contained in common autologous proteins (“Tregitopes”), compositions containing such Tregitopes, and for methods related to their preparation and use.

### SUMMARY

The aim of the present disclosure is to provide novel, therapeutic regulatory T cell epitope compositions. Such compositions include one or more of e.g., polypeptides (which may be termed herein as “Treg activating regulatory T-cell epitope”, “Tregitope”, “Tregitope peptide”, or “T-cell epitope polypeptide”) having a sequence comprising, consisting of, or consisting essentially of one or more of SEQ ID NOS: 1-73 (and/or fragments and variants thereof), and optionally 1 to 12 additional amino acids distributed in any ratio on the N terminus and/or C-terminus of the polypeptide of SEQ ID NOS: 1-73 (in aspects, the polypeptides may be isolated, synthetic, or recombinant) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein, and use of the same, e.g., to suppress an immune response in the body or more specifically to suppress an immune response in the body caused by the administration of a therapeutic agent to treat a medical condition.

The selective engagement and activation of naturally occurring T_{Regs} (in aspects, including natural T_{Regs} and/or adaptive T_{Regs}) through the use of Tregitope compounds and compositions and Tregitope-antigen compositions as disclosed herein, is therapeutically valuable as a means of treatment for any disease or condition marked by the presence of an unwanted immune response. Examples of such an unwanted immune response include the following: Autoimmune disease such as type 1 diabetes, MS, Lupus, and RA; Transplant related disorders such as Graft vs. Host disease (GVHD) and Host vs. Graft disease (HVGD); Allergic reactions; Immune rejection of biologic medicines such as monoclonal antibodies; the management of immune response targeting replacement proteins, e.g., but not limited to, Insulin, acid alpha glucosidase (“GAA” or lysosomal acid-glucosidase “LYAG”)) supplements; the management of immune response targeting therapeutic toxins such as Botulinum toxin; and the management of immune response to infectious disease whether acute or chronic.

The present disclosure harnesses the functions of naturally occurring T_{Regs} (in aspects, including natural T_{Regs} and/or adaptive T_{Regs}), and in particular aspects, those cells that already regulate immune responses to foreign and self-proteins in the periphery (pre-existing or natural T_{Regs}). In aspects, the present disclosure provides Tregitope compounds and compositions, with such compositions including one or more of, e.g., polypeptides having a sequence comprising, consisting of, or consisting essentially of one or more of SEQ ID NOS: 1-73 (and/or fragments and variants thereof), and optionally 1 to 12 additional amino acids distributed in any ratio on the N terminus and/or C-terminus of the polypeptide of SEQ ID NOS: 1-73 as disclosed herein; nucleic acids, expression cassettes, plasmids, expression vectors, recombinant viruses, or cells as disclosed herein; chimeric or fusion polypeptide compositions as disclosed herein; and/or pharmaceutical compositions or formulations as disclosed herein. T_{Reg} activating regulatory T-cell epitopes may be hereinafter referred to as “a” or “the” “Tregitope” or “Tregitopes”. In aspects, a Tregitope compound or composition of the present disclosure may be either covalently bound, non-covalently bound, or in admixture with a specific target antigen.

In aspects, the present disclosure is directed to a Tregitope polypeptide having a sequence comprising, consisting of, or consisting essentially of one or more of SEQ ID NOS: 1-73, or fragments and variants thereof. The phrase “consisting essentially of” is intended to mean that a polypeptide according to the present disclosure, in addition to the sequence according to any of SEQ ID NOS: 1-73 or a variant thereof, contains additional amino acids or residues that may be present at either terminus of the peptide and/or on a side chain that are not necessarily forming part of the peptide that functions as an MHC ligand and provided they do not substantially impair the activity of the peptide to function as a Tregitope. In aspects, an isolated, synthetic, or recombinant polypeptide (Treg activating regulatory T-cell epitope, Tregitope, Tregitope peptide, or T-cell epitope polypeptide) comprises, consists of, or consists essentially of one or more of SEQ ID NOS: 1-6. The polypeptides of the present disclosure may be isolated, synthetic, and/or recombinant, and may comprise post-transcriptional modifications such as glycosylation, added chemical groups, etc. In aspects, the peptides or polypeptides can be either in neutral (uncharged) or salt forms, and may be either free of or include modifications such as glycosylation, side chain oxidation, or phosphorylation. In certain aspects, the Tregitopes can be capped with an N-terminal acetyl and/or C-terminal amino group.

In aspects, the instant disclosure is directed to a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73. In aspects, the instant disclosure is directed to a peptide or polypeptide have a core amino acid sequence comprising, consisting of, or consisting essentially of one or more peptides or polypeptides having an amino acid sequence of SEQ ID NOS: 1-73, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide having a core sequence comprising, consisting of, or consisting essentially of one or more peptides or polypeptides having an amino acid sequence of SEQ ID NOS: 1-73 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to the same HLA molecule (i.e., retain MHC binding propensity) as said polypeptide core sequence without said flanking amino acids. In aspects, said polypeptide with the flanking amino acids is still able to bind to the same HLA molecule (i.e., retain MHC binding propensity) and retain the same TCR specificity as said polypeptide core sequence without said flanking amino acids. In aspects, said polypeptide with the flanking amino acids is still able to bind to a same HLA molecule (i.e., retain MHC binding propensity) and/or retain the same TCR specificity, and/or retain Tregitope activity, as said polypeptide core sequence without said flanking amino acids. In aspects, said flanking amino acid sequences are those that also flank the peptides or polypeptides included therein in the naturally occurring protein (e.g., as found in human acid alpha-glucosidase (“GAA”) or lysosomal alpha-glucosidase (“LYAG”)). In aspects, said flanking amino acid sequences as described herein may serve as a MHC stabilizing region. The use of a longer peptide may allow endogenous processing by patient cells and may lead to more effective antigen presentation and induction of T cell responses. In aspects, the peptides or polypeptides can be either in neutral (uncharged) or salt forms, and may be either free of or include modifications such as glycosylation, side chain oxidation, or phosphorylation. In certain aspects, the Tregitopes can be capped with an n-terminal acetyl and/or c-terminal amino group.

In aspects, the instant disclosure is directed to a polypeptide comprising an amino acid sequence having at least 75%, 80%, 85%, 90%, or 95% homology to any one of SEQ ID NOS: 1-73 (and/or fragments thereof), wherein said polypeptide is still able to bind to a same HLA molecule (i.e., retain MHC binding propensity) and/or retain the same TCR specificity, and/or retain regulatory T cell stimulating or suppressive activity.

In aspects, the present disclosure is directed to a concatemeric polypeptide or peptide that comprises at one or more of the instantly-disclosed polypeptides or peptides (e.g., but not limited to, a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects a concatemeric peptide is composed of 1 or more, 2 or more, 3 or more, 4 or more, 5 or more 6 or more 7 or more, 8 or more, 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage sensitive sites, adjacent to their N and/or C terminal end. In such a concatemeric peptide, two or more of the peptide epitopes may have a cleavage sensitive site between them. Alternatively two or more of the peptide epitopes may be connected directly to one another or through a linker that is not a cleavage sensitive site. In aspects, the instantly-disclosed concatemeric polypeptide or peptide sequences do not correspond to a naturally occurring sequence, i.e., each of the one or more of the instantly-disclosed polypeptides or peptides (e.g., but not limited to, a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) are linked, fused, or joined together (e.g., fused in-frame, chemically-linked, or otherwise bound) to an additional peptide or polypeptide (which may be one or more of the instantly-disclosed peptides) in such a fashion such that the overall concatemeric polypeptide does not correspond to a naturally occurring GAA or LYAG sequence. In aspects of above-described concatemeric peptides or polypeptides, the concatemeric peptides or polypeptides may be isolated, synthetic, or recombinant. In aspects, the concatemeric peptides or polypeptides can be either in neutral (uncharged) or salt forms, and may be either free of or include modifications such as glycosylation, side chain oxidation, or phosphorylation. In certain aspects, the concatemeric polypeptides can be capped with an n-terminal acetyl and/or c-terminal amino group.

In aspects, one or more peptides or polypeptides or concatemeric polypeptides of the instant disclosure (e.g., but not limited to, a peptide or polypeptide comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73) may be joined to, linked to (e.g., fused in-frame, chemically-linked, or otherwise bound), and/or inserted into a heterologous polypeptide. In aspects, the one or more peptides or polypeptides of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide.

In aspects, the present disclosure is directed to polypeptide (which, in aspects, may be an isolated, synthetic, or recombinant) having a sequence comprising one or more of SEQ ID NOS: 1-73 (and fragments or variants thereof), wherein said one or more of SEQ ID NOS: 1-73 is not naturally included in the polypeptide and/or said one or more of SEQ ID NOS: 1-73 is not located at its natural position in the polypeptide. In aspects, a polypeptide (which, in aspects, may be an isolated, synthetic, or recombinant) comprises one or more of SEQ ID NOS: 1-73, wherein said polypeptide does not comprise human acid alpha-glucosidase (“GAA”) or lysosomal alpha-glucosidase (“LYAG”) or a fragment thereof. In aspects, if a polypeptide does comprise human GAA/LYAG or a fragment thereof, then said one or more of SEQ ID NOS: 1-73 is not located in its natural position in the human GAA/LYAG or a fragment thereof. In aspects, one or more Tregitopes of the instant disclosure (which, in aspects, may be an isolated, synthetic, or recombinant) having a sequence comprising one or more of SEQ ID NOS: 1-73 (and fragments or variants thereof), and optionally 1 to 12 additional amino acids distributed in any ratio on the N terminus and/or C-terminus of the polypeptide of SEQ ID NOS: 1-73, may also be fused to or inserted internally within (e.g., but not limited to, site directed mutagenesis or other recombinant techniques) a GAA/LYAG molecule or a recombinant human GAA (“rhGAA” or alglucosidase alfa) supplement (or fragments thereof), such as in instances where the Tregitope is not located in its natural position within the GAA/LYAG molecule or wherein the GAA/LYAG molecule is missing such a Tregitope (e.g., if a particular the GAA/LYAG molecule has a mutated or missing corresponding section).

In aspects, the present disclosure is directed to a chimeric or fusion polypeptide composition (which in aspects may be isolated, synthetic, or recombinant) comprising one or more peptides, polypeptides, or concatemeric peptides of the present disclosure. In aspects, a chimeric or fusion polypeptide composition of the present disclosure comprises one or more peptides, polypeptides, or concatemeric peptides of the present disclosure joined to, linked to (e.g., fused in-frame, chemically-linked, or otherwise bound), and/or inserted into a heterologous polypeptide. In aspects, the one or more polypeptides (Treg activating regulatory T-cell epitope, Tregitope, Tregitope peptide, or T-cell epitope polypeptide) of the present disclosure may be inserted into the heterologous polypeptide, may be added to the C-terminus, and/or added to the N-terminus of the heterologous polypeptide. In aspects, a chimeric or fusion polypeptide composition of the present disclosure comprises a peptide, polypeptide, and/or concatemeric peptide of the present disclosure, wherein said peptide, polypeptide, and/or concatemeric peptide having a sequence that is not naturally included in the heterologous polypeptide and/or is not located at its natural position in the heterologous polypeptide. In aspects of the above chimeric or fusion polypeptide compositions, the one or more polypeptides (Treg activating regulatory T-cell epitope, Tregitope, Tregitope peptide, or T-cell epitope polypeptide) of the present disclosure have a sequence comprising, consisting of, or consisting essentially of one or more of SEQ ID NOS: 1-73. In aspects, the one or more isolated, synthetic, or recombinant polypeptides (Treg activating regulatory T-cell epitope, Tregitope, Tregitope peptide, or T-cell epitope polypeptide) of the present disclosure comprises, consists of, or consists essentially of a sequence one or more of SEQ ID NOS: 1-6, or 1-8. In aspects of the instantly-disclosed chimeric or fusion polypeptide compositions, the one or more polypeptides comprise, consist, or consist essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73. In aspects of the chimeric or fusion polypeptide compositions, the one or more of SEQ ID NOS: 1-73 may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition of the present disclosure comprises a polypeptide, said polypeptide having a sequence comprising one or more of SEQ ID NOS: 1-73 of the present disclosure, wherein said one or more of SEQ ID NOS: 1-73 is not naturally included in the polypeptide and/or said of one or more of SEQ ID NOS: 1-73 is not located at its natural position in the polypeptide. In aspects, the one or more of SEQ ID NOS: 1-73 of the present disclosure can be joined, linked to (e.g., fused in-frame, chemically-linked, or otherwise bound), and/or inserted into the polypeptide. In aspects of the instantly-disclosed chimeric or fusion polypeptide compositions, the heterologous polypeptide or polypeptide comprises a biologically active molecule. In aspects, the biologically active molecule is selected from the group consisting of an immunogenic molecule, a T cell epitope, a viral protein, and a bacterial protein. In aspects, the one or more of Tregitopes of the present disclosure can be joined or linked to (e.g., fused in-frame, chemically linked, or otherwise bound) to a small molecule, drug, or drug fragment. In aspects of above-described chimeric or fusion polypeptide compositions, the chimeric or fusion polypeptides may be isolated, synthetic, or recombinant. In aspects, the chimeric or fusion polypeptides can be either in neutral (uncharged) or salt forms, and may be either free of or include modifications such as glycosylation, side chain oxidation, or phosphorylation.

In aspects, the present disclosure is directed to a nucleic acid (e.g., DNAs, such as cDNA, or RNAs, such as mRNA), which in aspects may be isolated, synthetic, or recombinant, encoding one or more peptides, polypeptides, concatemeric peptides, and/or chimeric or fusion polypeptides as described herein. For example, in aspects, the instant disclosure is directed to a nucleic acid encoding a peptide or polypeptide comprising, consisting of, or consisting essentially of one or more peptides or polypeptides having an amino acid sequence of SEQ ID NOS: 1-73 (and/or fragments or variants thereof), and optionally 1 to 12 additional amino acids distributed in any ratio on the N-terminus and/or C-terminus of the polypeptide of SEQ ID NOS: 1-73. In aspects of the nucleic acids encoding one or more polypeptides (Treg activating regulatory T-cell epitope, Tregitope, Tregitope peptide, or T-cell epitope polypeptide) of the present disclosure or chimeric or fusion polypeptide compositions of the present disclosure, the one or more polypeptides or recombinant chimeric or fusion polypeptide compositions have a sequence comprising, consisting of, or consisting essentially of one or more of SEQ ID NOS: 1-6. In aspects, the present disclosure is directed to expression cassettes, plasmids, expression vectors, recombinant viruses, or cells comprising a nucleic acid as described herein. In aspects, the present disclosure is directed to a cell or vaccine comprising such a vector as described. In aspects, the present disclosure is directed to a cell comprising a vector of the present disclosure.

In aspects, the instant disclosure is directed to a pharmaceutical composition, the pharmaceutical composition comprising a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein) and a pharmaceutically acceptable carrier, excipient, and/or adjuvant. Another aspect is directed to a pharmaceutical composition, the pharmaceutical composition comprising one or more nucleic acids encoding one or more peptides, polypeptides, concatermic peptides, and/or chimeric or fusion polypeptides as disclosed herein, and a pharmaceutically acceptable carrier, excipient, and/or adjuvant. In aspects, the one or more nucleic acids encoding said peptides or polypeptides are DNA, RNA, or mRNA. In aspects of the above-described pharmaceutical compositions, the composition comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 100, at least 200, at least 300, at least 400, at least 500, at least 600, at least 700, at least 800, at least 900, or at least 1000, peptides, polypeptides, and/or concatemeric peptides, as disclosed herein, including every value or range therebetween.

In aspects, the present disclosure is directed to a method of stimulating, inducing, and/or expanding regulatory T-cells (in aspects, naturally occurring T_{Regs}, including natural T_{Regs} and/or adaptive T_{Regs}) in a subject in need thereof and/or suppressing an immune response in a subject in need thereof by administering to the subject a therapeutically effect amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein). In aspects, the subject is a human.

In aspects, the present disclosure is directed to a method of treating or preventing a medical condition in a subject in need thereof comprising administering a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein). In aspects, the medical condition is selected from the group consisting of: an allergy, an autoimmune disease, a transplant related disorder, graft versus host disease, a blood clotting disorder, an enzyme or protein deficiency disorder, a hemostatic disorder, cancer, infertility; and a viral, bacterial or parasitic infection. In another embodiment, the medical condition is hemophilia A, B, or C. In aspects, the subject is a human.

In aspects, the present disclosure is directed to a method of stimulating, inducing, and/or expanding regulatory T-cells (e.g., naturally occurring TRegs (in aspects, including natural TRegs and/or adaptive TRegs)) to suppress an immune response in a subject in need thereof by administering to the subject a therapeutically effect amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein). In aspects, the immune response is the result of one or more therapeutic treatments with at least one therapeutic protein, treatment with a vaccine or treatment with at least one antigen. In a particular embodiment, the immune response is the result of one or more therapeutic treatments with a recombinant human GAA (“rhGAA” or alglucosidase alfa) supplement. Thus, the administration of one or more Tregitope compounds and compositions of the present disclosure can be used to prevent the development of, or terminate, an already-established immune response to establish tolerance induction to rhGAA (and any GAA supplements) in patients suffering from Pompe Disease. In another aspect, the administration of a Tregitope compound or composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method for expanding a population of regulatory T cells, comprising: (a) providing a biological sample from a subject; and (b) isolating regulatory T-cells from the biological sample; (c) contacting the isolated regulatory T-cells with an effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), under conditions wherein the T-regulatory cells increase in number to yield an expanded regulatory T-cell composition, thereby expanding the regulatory T-cells in the biological sample; and, additionally, (d) returning the sample to the patient in need of treatment.

In aspects, the present disclosure is directed to a method for stimulating regulatory T cells in a biological sample, comprising: (a) providing a biological sample from a subject; (b) isolating regulatory T-cells from the biological sample; (c) contacting the isolated regulatory T-cells with an effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), under conditions wherein the T-regulatory cells are stimulated to alter one or more biological function, thereby stimulating the regulatory T-cells in the biological sample; and, additionally, (d) returning cells to the patient in need of treatment.

In aspects, the present disclosure is directed to a method for repressing/suppressing an immune response in a subject, comprising administering a therapeutically effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), wherein the Tregitope compound or composition represses/suppresses the immune response. In aspects, the Tregitope compound or composition represses/suppresses an innate immune response. In aspects, the Tregitope compound or composition represses/suppresses an adaptive immune response. In aspects, the Tregitope compound or composition represses/suppresses an effector T cell response. In aspects, the Tregitope compound or composition represses/suppresses a memory T cell response. In aspects, the Tregitope compound or composition represses/suppresses helper T cell response. In aspects, the Tregitope compound or composition represses/suppresses B cell response. In aspects, the Tregitope compound or composition represses/suppresses an NKT cell (natural killer T cell) response. In another aspect, the administration of a Tregitope compound or composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method of suppressing an immune response, specifically an antigen specific immune response in a subject, through the administration of a therapeutically effective amount of a Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein), wherein said Tregitope compound or composition activates naturally occurring T_{Regs} (in aspects, including natural T_{Regs} and/or adaptive T_{Regs}, and in aspects CD4⁺/CD25⁺/FoxP3⁺ regulatory T-cells) or suppresses the activation of CD4⁺ T-cells, the proliferation of CD4⁺ and/or CD8⁺ T-cells, and/or suppresses the activation or proliferation of β-cells or NKT Cells. In aspects, a Tregitope compound or composition of the present disclosure may be either covalently bound, non-covalently bound, or in admixture with a specific target antigen. In aspects, an administered Tregitope compound or composition of the present disclosure that is covalently bound, non-covalently bound, or in admixture with a specific target antigen results in the diminution of immune response against the target antigen.

In aspects, the target antigen may be an autologous protein or protein fragment. In aspects, the target antigen may be an allergen. In aspects, the target antigen may allogenic protein or protein fragments. In aspects, the target antigen may be a biologic medicine or fragments thereof. In aspects, the target antigen is GAA/LYAG or a recombinant GAA, such as a recombinant human GAA (“rhGAA” or alglucosidase alfa) supplement. In aspects, the suppressive effect is mediated by natural T_{Regs}. In aspects, the suppressive effect is mediated by adaptive T_{Regs}. In aspects, the one or more Tregitope included in the in the Tregitope compound or composition of the instant disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) suppresses an effector T cell response. In aspects, the one or more Tregitopes of the presently-disclosed Tregitope compounds or compositions suppresses an innate immune response. In aspects, the one or more Tregitopes of the presently-disclosed Tregitope compounds or compositions suppresses an adaptive immune response. In aspects, the one or more Tregitopes of the presently-disclosed Tregitope compounds and compositions suppresses helper T cell response. In aspects, the one or more Tregitopes of the presently-disclosed Tregitope compounds and compositions suppresses a memory T cell response. In aspects, the one or more Tregitopes of the presently-disclosed Tregitope compounds and compositions suppresses B cell response. In aspects, the one or more Tregitopes of the presently-disclosed Tregitope compounds and compositions suppresses NKT cell response.

In aspects, the present disclosure is directed to a kit for preventing or treating a medical condition, in particular, for the suppression of an immune response in a subject, wherein the kit comprises a Tregitope compound or composition of the instant disclosure. In aspects, the kit may further comprise an effective amount of an antigen or allergen or therapeutic agent, such as a replacement protein or peptide.

Additionally, the present disclosure is directed to a method for decreasing the immunogenicity and/or increasing tolerogenicity of a polypeptide, which may be particularly useful when a polypeptide (such as GAA/LYAG or a recombinant GAA, such as a recombinant human GAA (“rhGAA” or alglucosidase alfa) supplement, (or fragments thereof)) serves as a therapeutic protein. In aspects, said method comprises insertion of one or more regulatory T cell epitopes (e.g., a peptide or polypeptide comprising, consisting of, or consisting essentially of one or more peptides or polypeptides having an amino acid sequence of SEQ ID NOS: 1-73 (and/or fragments or variants thereof), and optionally 1 to 12 additional amino acids distributed in any ratio on the N-terminus and/or C-terminus of the polypeptide of SEQ ID NOS: 1-73) into said polypeptide (e.g., GAA/LYAG or a recombinant GAA supplement (or fragments thereof). In aspects, the one or more regulatory T cell epitopes inserted into the GAA/LYAG or a recombinant GAA supplement (or fragments thereof) can suppress an antigen-specific immune response against the human GAA/LYAG molecule or replacement protein/supplement (or fragments thereof). In aspects, said one or more regulatory T cell epitopes may be fused to or inserted internally within (e.g., but not limited to, site directed mutagenesis or other recombinant techniques) a human GAA/LYAG or a recombinant GAA supplement (or fragments thereof), such as in instances where the Tregitope is not located in its natural position within the GAA/LYAG molecule or replacement protein/supplement (or fragments thereof) or wherein the GAA/LYAG molecule or replacement protein/supplement (or fragments thereof) thereof is missing such a Tregitope (e.g., if a particular GAA/LYAG molecule or replacement protein/supplement (or fragments thereof) has a mutated or missing corresponding section). In aspects, said insertion of the one or more regulatory T cell epitopes into the GAG/LYAG molecule or replacement protein/supplement (or fragments thereof) thereof comprises insertion of all or some of the amino acids of the one or more regulatory T cell epitopes. In aspects, said insertion of the one or more regulatory T cell epitopes into the GAG/LYAG molecule or replacement protein/supplement (or fragments thereof) thereof comprises insertion of some or all of the amino acids of the one or more regulatory T cell epitopes and removing one or more amino acids at the site of insertion of the regulatory T cell epitope amino acids. In aspects, said insertion of the one or more regulatory T cell epitopes into the GAG/LYAG molecule or replacement protein/supplement (or fragments thereof) comprises mutating the sequence of the GAG/LYAG molecule or replacement protein/supplement (or fragments thereof) thereof to include the one or more regulatory T cell epitopes (for example, but not limited to, introduction one or more point mutations into the antibody or fragment thereof by site-directed mutagenesis or other recombinant techniques). In aspects, said insertion of the one or more regulatory T cell epitopes into the GAG/LYAG molecule or replacement protein/supplement (or fragments thereof), which in aspects will introduce the one or more regulatory T cell epitope sequences, such that the previous immunogenicity of the sequence is decreased and the tolerogenicity of the new sequence is enhanced. In aspects, the number of said added one or more amino acids at the site of insertion of the regulatory T cell epitope amino acids need not correspond to the number of amino acids deleted from the sequence of the GAG/LYAG molecule or replacement protein/supplement (or fragments thereof).

In aspects, said insertion of one or more regulatory T cell epitopes into the GAG/LYAG molecule or replacement protein/supplement (or fragments thereof) results in decreasing the immunogenicity of the antibody or fragment thereof. In aspects, said insertion of one or more regulatory T cell epitopes into the GAG/LYAG molecule or replacement protein/supplement (or fragments thereof) thereof results in a increasing the tolerogenicity of the GAG/LYAG molecule or replacement protein/supplement (or fragments thereof). In aspects, the one or more regulatory T cell epitopes have a sequence comprising, consisting of, or consisting essentially of one or more of SEQ ID NOS: 1-73.

### BRIEF DESCRIPTION OF THE DRAWINGS

The present disclosure may be better understood with reference to the following figures.

The patent or application file contains at least one drawing executed in color. Copies of this patent or patent application publication with color drawing(s) will be provided by the Office upon request and payment of the necessary fee.

FIG. 1 is the cluster report for the Tregitope of SEQ ID NO: 2 and the 9-mers contained within SEQ ID NO: 2, including SEQ ID NOS: 9-22.

FIG. 2 is the JanusMatrix report for the Tregitope of SEQ ID NO: 2 and the 9-mers contained within SEQ ID NO: 2, including SEQ ID NOS: 9-22.

FIG. 3 is the cluster report for the Tregitope of SEQ ID NO: 1 and the 9-mers contained within SEQ ID NO: 1, including SEQ ID NOS: 9-17.

FIG. 4 is the JanusMatrix report for the Tregitope of SEQ ID NO: 2 and the 9-mers contained within SEQ ID NO: 1, including SEQ ID NOS: 9-17.

FIG. 5 is the cluster report for the Tregitope of SEQ ID NO: 3 and the 9-mers contained within SEQ ID NO: 3, including SEQ ID NOS: 53, 54 and 17-22.

FIG. 6 is the JanusMatrix report for the Tregitope of SEQ ID NO: 3 and the 9-mers contained within SEQ ID NO: 3, including SEQ ID NOS: 53, 54 and 17-22.

FIG. 7 is the cluster report for the Tregitope of SEQ ID NO: 4 and the 9-mers contained within SEQ ID NO: 4, including SEQ ID NOS: 23-29.

FIG. 8 is the JanusMatrix report for the Tregitope of SEQ ID NO: 4 and the 9-mers contained within SEQ ID NO: 4, including SEQ ID NOS: 23-29.

FIG. 9 is the cluster report for the Tregitope of SEQ ID NO: 5 and the 9-mers contained within SEQ ID NO: 5, including SEQ ID NOS: 3-38.

FIG. 10 is the JanusMatrix report for the Tregitope of SEQ ID NO: 5 and the 9-mers contained within SEQ ID NO: 5, including SEQ ID NOS: 3-38.

FIG. 11 is the cluster report for the Tregitope of SEQ ID NO: 6 and the 9-mers contained within SEQ ID NO: 6, including SEQ ID NOS: 39-52.

FIG. 12 is the JanusMatrix report for the Tregitope of SEQ ID NO: 6 and the 9-mers contained within SEQ ID NO: 6, including SEQ ID NOS: 39-52. Also noted are six additional potential cross-reactive peptides from the human proteome that share conserved TCR-facing residues.

FIG. 13 is an overview of JanusMatrix results for identified the Tregitopes of the instant disclosure. To determine cross-conservation with the human proteome, each of the clusters were screened using JanusMatrix (search database is human). In general, when comparing peptide epitopes in proteins to the human genome, JanusMatrix Human Homology Scores above two are considered significant; indicating an elevated level of conservation between the TCR-facing features of the input peptide or protein and the TCR-facing features of the proteins resident within the human genome. For fully human proteins such as GAA, we can extend that threshold to three, since we know each TCR motif will be found at least once (because the human genome reference database contains a copy of the input sequence. The thresholds used in this analysis to select peptides are: JMX scores 0—<3=Low Conservation; JMX scores ≥3 and <5=Elevated Conservation; and, JMX scores ≥5=High Conservation. Epitopes that were predicted to bind across multiple HLA alleles and had a JanusMatrix score >2.5. This definition is a typical characteristic of other IgG-derived peptides. JM score was lowered to 2.5 to obtain more candidates in this category. 11 peptides were identified. These fell into a class referred to as Category 1. Category 2 contained restricted binding (non promiscuous) epitopes that met the JMX threshold >5 (i.e. meaning that each frame must have at least 5 matches). Category 3 was the same as 2, except that it included peptides with high JMX conservation for DRB1*0401 only.

FIG. 14 shows the synthesized peptide sequences that fell within Category 1.

FIG. 15 shows the synthesized peptide sequences that fell within Category 2 and 3.

FIG. 16 shows the final identified sequences with their corresponding predicted binding to DRB1*00101, *0301, *0401, *0701, *0801, *0901, *1101, *1301, and *1501.5 of the peptides fell within Category 1 with good cross-binding and 1 (SEQ ID NO: 4) demonstrated restricted HLA DR binding to DRB*1301.

FIG. 17 shows the location of the identified peptides with respect to their positions within full-length GAA. 3 of the peptides are overlapping within at least part of the amino-terminal signal sequence.

FIG. 18 shows initial HLA binding results for SEQ ID NOS: 2, 5, 6, and 4. The results showed an unexpected high false positive for SEQ ID NO: 2 and a high false negative for SEQ ID NO: 4. As a result, the peptides at the amino terminus were re-designed for further testing.

FIG. 19 shows HLA DR binding for the re-designed amino terminus peptides. For the sequence commencing at position 15, the initial valine and cysteine residues were mutated to alanines.

FIG. 20 shows an outline foe the TTBSA (Tetanus Toxoid Bystander Assay) with timelines for labelling and treating marked, as well as characterization markers for gating in subsequent flow cytometry to evaluated the response to the tetanus toxoid.

FIG. 21 shows the initial gating strategy to obtain CD4 cells from the collected PBMCs after the TTBSA is complete.

FIG. 22 shows a continuation of gating strategy from FIG. 21 to assess for Tcell proliferation (CFSE^{low}), T cell activation (CD25^{hi}), T effector cells (CD25^{hi} and FoxP3^{low}) and T regulatory cells (CD127^{low}, CD25^{hi} and FoxP3^{hi}) FIG. 23 shows a series of TTBSA performed on first 20 candidate regulatory peptide. Table summarizes results across 6 healthy donors evaluated. Also shaded triangles (representing each patient allele) indicate the IC50 as determined by the HLA Binding experiments for each peptide. Further evaluation of a subset of peptides was repeated in to identify the final set of validated peptides. The arrows indicate the selected six, with the amino terminus redesigned peptides (SEQ ID NOS: 1 and 3) being added to further testing.

FIG. 24 shows the sequences of the selected 8 peptides for a second round of TTBSA, as well as the positive control sequence.

FIG. 25 shows that Tetanus Toxoid caused CD4 proliferation in the cells obtained from 5 donors, as well as the haplotypes, ages and genders of the cell donors.

FIG. 26 shows that the positive control (SEQ ID NO: 74) was able to successfully inhibit CD4 proliferation in all five donor cell lines.

FIG. 27 shows effects of each of the six test peptides on CD4 proliferation in the TTBSA on cells from donor EV302. SEQ ID NOS: 7 and 8, as well as 4, showed little effect on CD4 proliferation in the cells from this donor.

FIG. 28 shows the ratio of T regulatory cells to T effector cells generated in donor EV302, with SEQ ID NOS: 1-3, 5 and 6 all showing good ratios as compared to the positive control.

FIG. 29 shows effects of each of the six test peptides on CD4 proliferation in the TTBSA on cells from donor EV256. SEQ ID NOS: 7 and 8, as well as 3 and 4, showed little effect on CD4 proliferation in the cells from this donor.

FIG. 30 shows the ratio of T regulatory cells to T effector cells generated in donor EV256, with SEQ ID NOS: 1 and 6 showing the best ratios as compared to the positive control.

FIG. 31 shows a summary table of the overall significance of response in the eight peptides on CD4 T cell proliferation. SEQ ID NOS: 7 and 8 showed little effect in all donors, while the other six produced good decreases.

FIG. 32 shows a dose response curve of inhibition of CD4 T cell proliferation with increasing concentrations of SEQ ID NO: 1 in a TTBSA across all five donor cells.

FIG. 33 shows a dose response curve of inhibition of CD4 T cell proliferation with increasing concentrations of SEQ ID NO: 2 in a TTBSA across all five donor cells.

FIG. 34 shows a dose response curve of inhibition of CD4 T cell proliferation with increasing concentrations of SEQ ID NO: 3 in a TTBSA across all five donor cells.

FIG. 35 shows a dose response curve of inhibition of CD4 T cell proliferation with increasing concentrations of SEQ ID NO: 4 in a TTBSA across all five donor cells.

FIG. 36 shows a dose response curve of inhibition of CD4 T cell proliferation with increasing concentrations of SEQ ID NO: 5 in a TTBSA across all five donor cells.

FIG. 37 shows a dose response curve of inhibition of CD4 T cell proliferation with increasing concentrations of SEQ ID NO: 6 in a TTBSA across all five donor cells.

### DETAILED DESCRIPTION OF THE INVENTION

### General

The adaptive immune cascade begins when soluble protein antigens are taken up by Antigen Presenting Cells (APCs) and processed through the Class II antigen presentation pathway. Protein antigens in the Class II presentation pathway are degraded by various proteases found in the Endoplasmic Reticulum. Some of the resulting protein fragments are bound to Class II MHC molecules. Peptide-loaded MHC molecules are trafficked to the cell surface where they are interrogated by CD4+ T cells. Peptide fragments that are capable of binding to an MHC molecule and mediating the cell to cell interaction between APC and circulating T cells are referred to as T cell epitopes. Recognition of these peptide-MHC complexes by CD4+ T cells can lead to either an immune activating or immune suppressive response based on the phenotype of the responding T cells and the local cytokine/chemokine milieu. In general, engagement between the MHC/peptide complex and the T cell receptor (TCR) of T effector cells leads to activation and the subsequent secretion of pro-inflammatory cytokines such as IL-4, and IFN-γ. On the other hand, the activation of natural T regulatory cells (T_{Regs}) leads to the expression of the immune suppressive cytokines IL-10 and TGF-β, among others (Shevach E, (2002), Nat Rev Immunol, 2(6):389-400). These cytokines act directly on nearby effector T cells leading in some cases to anergy or apoptosis. In other cases, regulatory cytokines and chemokines convert effector T cells to T regulatory phenotypes; this process is referred here as “induced” or “adaptive” tolerance. T cell epitopes that are capable of binding to MHC molecules and engaging and/or activating circulating naturally occurring T_{Regs} (in aspects, including natural T_{Regs} and/or adaptive T_{Regs}), are referred to as Tregitopes. In aspects, the instantly-disclosed Tregitopes are T cell epitope clusters, which are epitopes capable of binding to multiple MHC alleles and multiple TCRs.

Initial self/non-self discrimination occurs in the thymus during neonatal development where cortical and medullary epithelial cells express specific self-protein epitopes to immature T cells. T cells recognizing self-antigens with high affinity are deleted, but autoreactive T cells with moderate affinity sometimes avoid deletion and can be converted to so called natural regulatory T cells (T_{Reg}) cells. These natural T_{Reg} cells are exported to the periphery and help to control a latent autoimmune response. Natural regulatory T cells are a critical component of immune regulation and self-tolerance.

Self-tolerance is regulated by a complex interplay between T cells, B cells, cytokines and surface receptors. T regulatory immune responses counterbalance T effector immune response to protein antigens (whether self or foreign). A tilt of the balance toward the autoreactive side, either by increasing the number or function of autoreactive T effector cells or by diminishing the number or function of T regulatory cells, is manifested as autoimmunity.

A second form of tolerance occurs in the periphery where mature T cells are converted to an ‘adaptive’ T_{Reg} phenotype upon activation via their T cell receptor in the presence of IL-10 and TGF-β, usually supplied by bystander T regulatory cells. The possible roles for these ‘adaptive’ T_{Reg} cells include dampening immune response following the successful clearance of an invading pathogen, controlling excessive inflammation caused by an allergic reaction, controlling excessive inflammation caused by low level or chronic infection, or possibly controlling inflammatory response targeting beneficial symbiotic bacteria and viruses. ‘Adaptive’ T_{Regs} may also play a role in suppressing immune response targeting human antibodies that have undergone somatic hypermutation (Chaudhry A et al., (2011), Immunity, 34(4):566-78).

T_{Reg} cells are also instrumental in B cell tolerance. B cells express a single low affinity Fc receptor, FcγRIIB on their cell surface (Ravetch J V et al., (1986), Science, 234(4777):718-25). This receptor contains the immunoreceptor tyrosine-based inhibition motif sequence (ITIM) in its cytoplasmic domain. Co-ligation of FcγRIIB and the B-cell receptor (BCR) by immune complexes act to trigger the tyrosine phosphorylation of the ITIM leading to the recruitment of the inositol phosphatase, SHIP, which inhibits BCR-triggered proliferation by interfering with the activation of MAP kinases and blocks phagocytosis by the dissociation of Burton's tyrosine kinase (Btk) from the cell membrane, which inhibits calcium influx into the cell. FcγRIIB can also induce apoptosis independent of the ITIM. Upon homo-aggregation of FcγRIIB by ICs, the association of Btk with the cell membrane is enhanced, thereby triggering an apoptotic response (Pearse R, et al., (1999), Immunity, 10(6):753-60). Expression of FcγRIIB is highly variable and cytokine dependent. IL-4 and IL-10, which are expressed by activated Th2 and T_{Reg} cells, have been shown to act synergistically to enhance FcγRIIB expression (Joshi T et al., (2006), Mol Immuno., 43(7):839-50), thus aiding in the suppression of a humoral response.

It is possible to exploit Tregitope specific T_{Reg} cells to suppress unwanted immune responses and also to induce adaptive T_{Reg} to co-delivered proteins. This discovery has implications for the design of therapeutic regimens and antigen-specific therapies for transplantation, protein therapeutics, allergy, chronic infection, autoimmunity and vaccine design. Administration of a drug, a protein, or an allergen in conjunction with Tregitopes, including a Tregitope compound or composition of the present disclosure (including one or more of e.g., polypeptides (which may be termed herein as “T_{reg} activating regulatory T-cell epitope”, “Tregitope”, or “T-cell epitope polypeptide”) having a sequence comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) can suppress an effector immune response. Tregitopes, including Tregitope compounds and compositions of the present disclosure, can be used to deliberately manipulate the immune system toward tolerance.

The Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) are useful in the selective engagement and activation of regulatory T cells. It is demonstrated herein that certain naturally occurring T_{Reg}s (in aspects, including natural T_{Reg}s and/or adaptive T_{Regs}), can be engaged, activated, and/or applied to the suppression of unwanted immune responses in both systemic and limited, disease specific, contexts. In aspects, the Tregitope compounds and compositions of the present disclosure can be used to engage and activate pre-existing populations of regulatory T cells to suppress an immune response caused by enzyme replacement therapy (ERT) of human acid alpha-glucosidase (GAA) that is used to treat patients suffering from Pompe disease.

Despite extensive efforts, with few exceptions, the antigen specificity of natural T_{Reg}s, and more importantly natural T_{Reg}s circulating in clinically significant volumes, is unknown. Presented herein is a demonstration that certain human proteins circulating in the blood steam, such as immunoglobulins or the serum proteins GAA (or lysosomal alpha-glucosidase (LYGA)), contain T cell epitopes that relate to naturally occurring populations of regulatory T cells (in aspects, including natural T_{Regs} and/or adaptive T_{Regs}). In the course of normal immune surveillance, these proteins are taken up by professional APCs, such as dendritic cells or macrophages, and degraded. During the degradation process, some of the epitopes contained in these proteins are bound to MHC molecules, transported to the cell surface presented to regulatory T cells. Those cells, once activated by the APC, release cytokines and chemokines help to suppress autoimmune responses that would otherwise hinder the function of the extra cellular proteins.

By using the Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) to selectively activate naturally occurring T_{Regs} (in aspects, including natural T_{Regs} and/or adaptive T_{Regs}), it is herein shown that the Tregitope compound and compositions of the present disclosure can be used to suppress a variety of unwanted immune responses. In its simplest form, systemic application of the Tregitope compounds and compositions of the present disclosure can be used as a generalized immune suppressant useful for controlling severe autoimmune reactions such as, for example, MS flare-ups, allergic reactions, transplant reactions, or uncontrolled response to infection.

In a more controlled application, for example but not limited to, topically applied to joints affected by rheumatoid arthritis (RA), the Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) can be used to suppress localized autoimmune responses. In a targeted application, such as might be achieved through the fusion, bonding or admixture of the Tregitope compounds and compositions of the present disclosure to certain other T cell epitopes, the Tregitope compounds and compositions can suppress highly specific immune reactions to the fused, bonded, or admixed T cell epitopes while leaving the balance of the immune system intact. For example, through the delivery of a Tregitope compound or composition of the present disclosure fused to an autoimmune antigen such as insulin, an allergen such as Brazil nut antigen, or an antigenic protein such as an antibody (which can be IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody) or replacement enzyme, the immune system can be trained to “tolerate” the co-delivered antigen by, e.g., inducing naturally occurring T_{Regs} (in aspects, including natural T_{Regs} and/or adaptive T_{Regs}) and/or converting the phenotype of responding effector T cells to that of adaptive regulatory T cells.

In certain aspects, the Tregitope compounds and compositions of the present disclosure (including one or more of e.g., polypeptides (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) can be used to suppress an immune response caused by recombinant human GAA (rhGAA) or alglucosidase alfa as ERT to treat patients with Pompe disease. For example, rhGAA can be administered with the Tregitope compounds and compositions of the present disclosure (e.g. but not limited to, through fusion, bonding, or admixture of the rhGAA with the Tregitope compounds and compositions of the present disclosure, or insertion and/or linkage of the Tregitope(s) of the invention into the rhGAA sequence), with the Tregitope compounds and compositions of the present disclosure suppressing an immune response targeting the rhGAA. Such immune reprogramming could reduce or eliminate immune response targeting the rhGAA ERT used to treat Pompe disease, while leaving the balance of the immune system intact.

As stated above, the Tregitopes of the present disclosure are derived from circulating extracellular proteins. To be useful, these Tregitopes should be true T cell epitopes (i.e., capable of binding to both MHC molecules and TCRs). In aspects, the Tregitopes should be related to a pre-existing population of regulatory T cells that is sufficiently large to have a therapeutic effect. T cell epitope clusters, which are epitopes capable of binding to multiple MHC alleles and multiple TCRs, are key to satisfying this latter qualification.

More particularly, the Tregitopes of the present disclosure are components of human GAA or LYAG. In their natural state, the Tregitopes of the present disclosure are capable of engaging and activating naturally occurring T_{Reg}s (in aspects, including natural T_{Regs} and/or adaptive T_{Reg}s) which prevent or terminate immune responses. These GAA peptides are highly related to fragments of GAA. In aspects, exposure of patients with Pompe disease or that are CRIM-negative (cross-reactive immunologic material) to the Tregitope compounds and compositions of the present disclosure (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) can prevent an immune response to ERT with rhGAA allowing treated patients the full benefit of ERT therapy. Additionally, treatment with Tregitope compounds and compositions of the present disclosure can expand corresponding naturally occurring T_{Reg} populations (in aspects, including natural T_{Reg}s and/or adaptive T_{Regs}), making them available to be activated by homologous peptides derived from GAA, thereby suppressing effector response targeting rhGGA. The instantly-disclosed treatments provides the following advantages:
1. Treatment with the Tregitope compounds and compositions of the present disclosure is highly antigen specific (e.g., treatment with the Tregitope compounds and compositions can, e.g., expand and/or stimulate corresponding naturally occurring T_{Reg} populations (in aspects, including natural T_{Reg}s and/or adaptive T_{Reg}s) in a highly antigen specific manner);
2. An efficient and less expensive treatment regimen when compared to current antigen specific therapies wherein patients are treated over a prolonged period of time with frequent high dose rhGAA/GAA ERT; and
3. A second line of defense treatment when high dose rhGAA/GAA treatment fails to induce immune tolerance in the treated patient.

In aspects, the present disclosure is directed to therapeutic Tregitope compounds and compositions (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) that are safely administered to a patient experiencing an autoimmune response. The mechanism of action of the claimed Tregitopes (and Tregitopes within said Tregitope compounds and compositions) is natural, supporting their efficacy and safety. In aspects, the present disclosure is directed to therapeutic Tregitope compounds and compositions that are safely administered to a Pompe disease patient in need of treatment, given that the Tregitopes of the instant disclosure are natural components of GAA, and as such, are naturally present in all humans.

In aspects, the present is directed to Tregitope compounds and compositions (e.g., one or more of: peptides or polypeptides as disclosed herein; concatemeric peptides as disclosed herein; chimeric or fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, recombinant viruses, cells as disclosed herein; and/or pharmaceutical compositions or formulations as disclosed herein) that include one or more of the regulatory Tregitopes disclosed in Tables 1-8, as well as fragments thereof, variants thereof, and fragments of such variants thereof, provided said fragments and/or variants retain MHC binding propensity and/or TCR specificity and/or regulatory T cell stimulating or suppressive activity. In certain aspects, the Tregitopes can be capped with an n-terminal acetyl and/or c-terminal amino group.

TABLE 1
SEQ ID NO: 1 and 9-mers thereof

| SEQ ID NO | Position Range | Sequence |
|---|---|---|
| 1 | 9-25 | SHRLLAVCALVSLATAA |
| 9 | (9-17) | SHRLLAVCA |
| 10 | (10-18) | HRLLAVCAL |
| 11 | (11-19) | RLLAVCALV |
| 12 | (12-20) | LLAVCALVS |
| 13 | (13-21) | LAVCALVSL |
| 14 | (14-22) | AVCALVSLA |
| 15 | (15-23) | VCALVSLAT |
| 16 | (16-24) | CALVSLATA |
| 17 | (17-25) | ALVSLATAA |

TABLE 2
SEQ ID NO: 2 and 9-mers thereof

| SEQ ID NO | Position Range | Sequence |
|---|---|---|
| 2 | 9-30 | SHRLLAVCALVSLATAALLGHI |
| 9 | (9-17) | SHRLLAVCA |
| 10 | (10-18) | HRLLAVCAL |
| 11 | (11-19) | RLLAVCALV |
| 12 | (12-20) | LLAVCALVS |
| 13 | (13-21) | LAVCALVSL |
| 14 | (14-22) | AVCALVSLA |
| 15 | (15-23) | VCALVSLAT |
| 16 | (16-24) | CALVSLATA |
| 17 | (17-25) | ALVSLATAA |
| 18 | (18-26) | LVSLATAAL |
| 19 | (19-27) | VSLATAALL |
| 20 | (20-28) | SLATAALLG |
| 21 | (21-29) | LATAALLGH |
| 22 | (22-30) | ATAALLGHI |

TABLE 3
SEQ ID NO: 3 and 9-mers thereof

| SEQ ID NO | Position Range | Sequence |
|---|---|---|
| 3 | 15-30 | AAALVSLATAALLGHI |
| 53 | (15-23) | AAALVSLAT |
| 54 | (16-24) | AALVSLATA |
| 17 | (17-25) | ALVSLATAA |
| 18 | (18-26) | LVSLATAAL |
| 19 | (19-27) | VSLATAALL |
| 20 | (20-28) | SLATAALLG |
| 21 | (21-29) | LATAALLGH |
| 22 | (22-30) | ATAALLGHI |

TABLE 4
SEQ ID NO: 4 and 9-mers thereof

| SEQ ID NO | Position Range | Sequence |
|---|---|---|
| 4 | 162-176 | KDILTLRLDVMMETE |
| 23 | (162-170) | KDILTLRLD |
| 24 | (163-171 | DILTLRLDV |
| 25 | (164-172) | ILTLRLDVM |
| 26 | (165-173) | LTLRLDVMM |
| 27 | (166-174) | TLRLDVMME |
| 28 | (167-175) | LRLDVMMET |
| 29 | (168-176) | RLDVMMETE |

TABLE 5
SEQ ID NO: 5 and 9-mers thereof

| SEQ ID NO | Position Range | Sequence |
|---|---|---|
| 5 | 307-323 | AHGVFLLNSNAMDVVLQ |
| 30 | (307-315) | AHGVFLLNS |
| 31 | (308-316) | HGVFLLNSN |
| 32 | (309-317) | GVFLLNSNA |
| 33 | (310-318) | VFLLNSNAM |
| 34 | (311-319) | FLLNSNAMD |
| 35 | (312-320) | LLSNNAMDV |
| 36 | (313-321) | LNSNAMDVV |
| 37 | (314-322) | NSNAMDVVL |
| 38 | (315-323) | SNAMDVVLQ |

TABLE 6
SEQ ID NO: 6 and 9-mers thereof

| SEQ ID NO | Position Range | Sequence |
|---|---|---|
| 6 | 896-917 | GAGLQLQKVTVLGVATAPQQVL |
| 39 | (896-904) | GAGLQLQKV |
| 40 | (897-905) | AGLQLQKVT |
| 41 | (898-906) | GLQLQKVTV |
| 42 | (899-907) | LQLQKVTVL |
| 43 | (900-908) | QLQKVTVLG |
| 44 | (901-909) | LQKVTVLGV |
| 45 | (902-910) | QKVTVLGVA |
| 46 | (903-911) | KVTVLGVAT |
| 47 | (904-912) | VTVLGVATA |
| 48 | (905-913) | TVLGVATAP |
| 49 | (906-914) | VLGVATAPQ |
| 50 | (907-915) | LGVATAPQQ |
| 51 | (908-916) | GVATAPQQV |
| 52 | (909-917) | VATAPQQVL |

TABLE 7
SEQ ID NO: 7 and 9-mers thereof

| SEQ ID NO | Position Range | Sequence |
|---|---|---|
| 7 | (39-53) | PRELSGSSPVLEETH |
| 55 | (39-47) | PRELSGSSP |
| 56 | (40-48) | RELSGSSPV |
| 57 | (41-49) | ELSGSSPVL |
| 58 | (42-50) | LSGSSPVLE |
| 59 | (43-51) | SGSSPVLEE |
| 60 | (44-52) | GSSPVLEET |
| 61 | (45-53) | SSPVLEETH |

TABLE 8
SEQ ID NO: 8 and 9-mers thereof

| SEQ ID NO | Position Range | Sequence |
|---|---|---|
| 7 | (27-46) | LGHILLHDFLLVPRELSGSS |
| 62 | (27-35) | LGHILLHDF |
| 63 | (28-36) | GHILLHDFL |
| 64 | (29-37) | HILLHDFLL |
| 65 | (30-38) | ILLHDFLLV |
| 66 | (31-39) | LLHDFLLVP |
| 67 | (32-40) | LHDFLLVPR |
| 68 | (33-41) | HDFLLVPRE |
| 69 | (34-42) | DFLLVPREL |
| 70 | (35-43) | FLLVPRELS |
| 71 | (36-44) | LLVPRELSG |
| 72 | (37-45) | LVPRELSGS |
| 73 | (38-46) | VPRELSGSS |

### Definitions

To further facilitate an understanding of the present invention, a number of terms and phrases are defined below. Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the term “biological sample” as refers to any sample of tissue, cells, or secretions from an organism.

As used herein, the term “transplantation” refers to the process of taking a cell, tissue, or organ, called a “transplant” or “graft” from one subject and placing it or them into a (usually) different subject. The subject who provides the transplant is called the “donor”, and the subject who received the transplant is called the “recipient”. An organ or graft transplanted between two genetically different subjects of the same species is called an “allograft”. A graft transplanted between subjects of different species is called a “xenograft”.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues. In aspects, an immune response includes a measurable cytotoxic T lymphocyte (CTL) response (e.g., against a virus expressing an immunogenic polypeptide) or a measurable B cell response, such as the production of antibodies, (e.g., against an immunogenic polypeptide). One of ordinary skill would know various assays to determine whether an immune response against a peptide, polypeptide, or related composition was generated, including use of the experiments and assays as disclosed in the Examples herein. Various B lymphocyte and T lymphocyte assays are well known, such as ELISAs, EliSpot assays, cytotoxic T lymphocyte CTL assays, such as chromium release assays, proliferation assays using peripheral blood lymphocytes (PBL), tetramer assays, and other cytokine production assays. See Benjamini et al. (1991), hereby incorporated by reference.

As used herein, the term “effective amount”, “therapeutically effective amount”, or the like of a composition, including Tregitope compounds and compositions of the present disclosure (including one or more of e.g., polypeptides (which may be termed herein as “T_{reg} activating regulatory T-cell epitope”, “Tregitope”, or “T-cell epitope polypeptide”) having a sequence comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) is a quantity sufficient to achieve a desired therapeutic and/or prophylactic effect, e.g., an amount that results in the prevention of, or a decrease in, the symptoms associated with a disease that is being treated. The amount of a composition of the present disclosure administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present invention can also be administered in combination with each other or with one or more additional therapeutic compounds.

As used herein, the term “regulatory T cell”, “Treg” or the like, means a subpopulation of T cells that suppress immune effector function, including the suppression or down regulation of CD4+ and/or CD8+ effector T cell (Teff) induction, proliferation, and/or cytokine production, through a variety of different mechanisms including cell-cell contact and suppressive cytokine production. In aspects, CD4+ Tregs are characterized by the presence of certain cell surface markers including but not limited to CD4, CD25, and FoxP3. In aspects, upon activation, CD4+ regulatory T cells secrete immune suppressive cytokines and chemokines including but not limited to IL-10 and/or TGFβ. CD4+ Tregs may also exert immune suppressive effects through direct killing of target cells, characterized by the expression upon activation of effector molecules including but not limited to granzyme B and perforin. In aspects, CD8+ Tregs are characterized by the presence of certain cell surface markers including but not limited to CD8, CD25, and, upon activation, FoxP3. In aspects, upon activation, regulatory CD8+ T cells secrete immune suppressive cytokines and chemokines including but not limited to IFNγ, IL-10, and/or TGFβ. In aspects, CD8⁺ Tregs may also exert immune suppressive effects through direct killing of target cells, characterized by the expression upon activation of effector molecules including but not limited to granzyme B and/or perforin.

As used herein, the term “T cell epitope” means an MHC ligand or protein determinant, 7 to 30 amino acids in length, and capable of specific binding to human leukocyte antigen (HLA) molecules and interacting with specific T cell receptors (TCRs). As used herein, in the context of a T cell epitope that is known or determined (e.g. predicted) to engage a T cell, the terms “engage”, “engagement” or the like means that when bound to a MHC molecule (e.g. human leukocyte antigen (HLA) molecules), the T cell epitope is capable of interacting with the TCR of the T cell and activating the T cell. Generally, T cell epitopes are linear and do not express specific three-dimensional characteristics. T cell epitopes are not affected by the presence of denaturing solvents. The ability to interact with T cell epitopes can be predicted by in silico methods (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A R et al., (2003), Vaccine, 21(27-30):4486-504, all of which are herein incorporated by reference in their entirety).

As used herein, the term “T-cell epitope cluster” refers to polypeptide that contains between about 4 to about 40 MHC binding motifs. In particular embodiments, the T-cell epitope cluster contains between about 5 to about 35 MHC binding motifs, between about 8 and about 30 MHC binding motifs, or between about 10 and 20 MHC binding motifs.

As used herein, the term “regulatory T cell epitope” (“Tregitope”) refers to a “T cell epitope” that causes a tolerogenic response (Weber C A et al., (2009), Adv Drug Deliv, 61(11):965-76) and is capable of binding to MHC molecules and engaging (i.e. interacting with and activating) circulating naturally occurring Tregs (in aspects, including natural Tregs and/or adaptive Tregs). In aspects, upon activation, CD4+ regulatory T cells secrete immune suppressive cytokines and chemokines including but not limited to IL-10 and/or TGF-β and/or TNF-α. CD4+ Tregs may also exert immune suppressive effects through direct killing of target cells, characterized by the expression upon activation of effector molecules including but not limited to granzyme B and perforin. In aspects, upon activation, regulatory CD8+ T cells secrete immune suppressive cytokines and chemokines including but not limited to IFNγ, IL-10, and/or TGFβ. In aspects, CD8+ Tregs may also exert immune suppressive effects through direct killing of target cells, characterized by the expression upon activation of effector molecules including but not limited to granzyme B and/or perforin. In aspects, the instantly disclosed Tregitopes are T cell epitope clusters, which are epitopes capable of binding to multiple MHC alleles and multiple TCRs.

As used herein, the term “immune stimulating T-cell epitope polypeptide” refers to a molecule capable of inducing an immune response, e.g., e.g., a humoral, T cell-based, or innate immune response. In aspects, an immune stimulating T-cell epitope polypeptide is human GAA/LYAG molecule or a rhGAA replacement protein or supplement.

As used herein, the term “B cell epitope” means a protein determinant capable of specific binding to an antibody. B cell epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

The term “subject” as used herein refers to any living organism in which an immune response is elicited. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

As used herein, the terms “the major histocompatibility complex (MHC)”, “MHC molecules”, “MHC proteins” or “HLA proteins” are to be understood as meaning, in particular, proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles. MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin. MHC molecules of class II consist of an α-chain and a β-chain and are capable of binding a peptide of about 12 to 25 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

As used herein, the term “MHC Ligand” means a polypeptide capable of binding to one or more specific MHC alleles. The term “HLA ligand” is interchangeable with the term “MHC Ligand”. Cells expressing MHC/Ligand complexes on their surface are referred to as “Antigen Presenting Cells” (APCs). Similarly, as used herein, the term “MHC binding peptide” relates to a peptide which binds to an MHC class I and/or an MHC class II molecule. In the case of MHC class 1/peptide complexes, the binding peptides are typically 8-10 amino acids long although longer or shorter peptides may be effective. In the case of MHC class II/peptide complexes, the binding peptides are typically 10-25 amino acids long and are in particular 13-18 amino acids long, whereas longer and shorter peptides may also be effective.

As used herein, the term “T Cell Receptor” or “TCR” refers to a protein complex expressed by T cells that is capable of engaging a specific repertoire of MHC/Ligand complexes as presented on the surface of APCs.

As used herein, the term “MHC Binding Motif” refers to a pattern of amino acids in a protein sequence that predicts binding to a particular MHC allele.

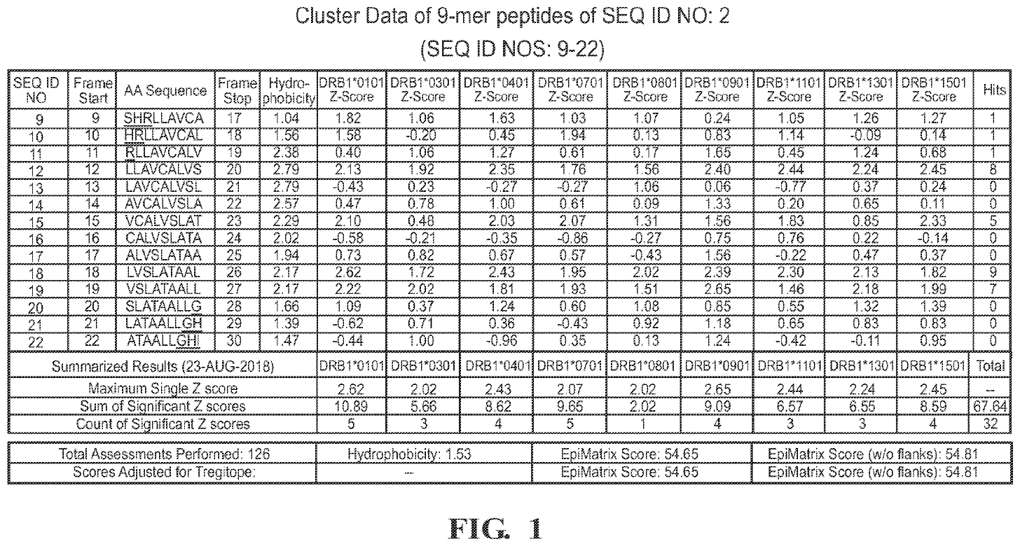

As used herein, the term “EpiBar™” refers to a single 9-mer frame that is predicted to bind to at least four different HLA alleles. A representative example of an immunogenic peptide that contains an EpiBar™ is shown below in FIG. 1. FIG. 1 depicts an example of an EpiBar and the EpiMatrix analysis of a SEQ ID NO: 2. It scores extremely high for all eight alleles in EpiMatrix. Its cluster score is 54.81. Cluster scores higher than 10 are considered to be significant. The band-like EpiBar pattern is characteristic of promiscuous epitopes. Z score indicates the potential of a 9-mer frame to bind to a given HLA allele. All scores in the top 5% are considered “hits”, while non hits (*) below 10% are masked in FIG. 1 for simplicity.

As used herein, the term “native Fc” refers to a molecule or sequence comprising the sequence of a non-antigen-binding fragment resulting from digestion of whole antibody, whether in monomeric or multimeric form, into which a peptide sequence may be added by insertion into or replacement of a loop region. The original immunoglobulin source of the native Fc is preferably of human origin and may be any of the immunoglobulins, although IgG1 and IgG2 are preferred Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

As used herein, the term “Immune Synapse” means the protein complex formed by the simultaneous engagement of a given T cell epitope to both a cell surface MHC complex and TCR.

The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide (e.g., a polypeptide comprising, consisting of, or consisting essentially of one or more of SEQ ID NOS: 1-73 or variants and fragments thereof, which in aspects may be isolated, synthetic, or recombinant) of the present disclosure, however, can be joined to, linked to, or inserted into another polypeptide (e.g., a heterologous polypeptide) with which it is not normally associated in a cell and still be “isolated” or “purified.” As used herein with respect to the one or more Tregitopes of the instant disclosure, the term “heterologous polypeptide” is intended to mean that the one or more Tregitopes is heterologous to, or not included naturally, in the heterologous polypeptide. For example, one or more Tregitopes of the instant disclosure may also be fused to or inserted internally within (e.g., but not limited to, site directed mutagenesis or other recombinant techniques) a human GAA sequence (SEQ ID NO: 101) or a variant thereof, such as in instances where the Tregitope is not located in its natural position within the human GAA sequence or where the subjects own GAA is missing such a Tregitope (e.g., if a particular patient has a mutated or missing corresponding section in their own expressed GAA)). Additionally, for example, one or more Tregitopes of the present disclosure (and/or one or more other Tregitopes, such as an IgG derived Tregitope as disclosed in U.S. Pat. No. 7,884,184, which is incorporated by reference in its entirety) can be linked to (e.g., fused in-frame, chemically-linked, or otherwise bound) and/or inserted into a heterologous polypeptide (e.g., a heterologous monoclonal antibody). Additionally, one or more Tregitopes of the present disclosure can be joined to, linked to, or inserted into another polypeptide wherein said one or more Tregitopes of the present disclosure is not naturally included in the polypeptide and/or said one or more Tregitopes of the present disclosure is not located at its natural position in the polypeptide. For example, in aspects, the one or more Tregitopes may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). In aspects, the one or more Tregitopes may be covalently bound to one or more internal conjugation site(s) in a Fc domain as disclosed in U.S. Pat. Nos. 8,008,453, 9,114,175, and/or 10,188,740 (each of which are herein incorporated by reference in their entirety). When a polypeptide is recombinantly produced, it can also be substantially free of culture medium, for example, culture medium represents less than about 20%, less than about 10%, or less than about 5% of the volume of the polypeptide preparation.

As used herein, a “concatemeric” peptide or polypeptide refers to a series of at least two peptides or polypeptides linked together. Such linkages may form of string-of-beads design. In aspects, each of the peptides or polypeptides of concatemeric polypeptide may optionally be spaced by one or more linkers, and in further aspects neutral linkers. The term “linker” may refer to a peptide added between two peptide domains such as epitopes or vaccine sequences to connect said peptide domains. In aspects, a linker sequence is used to reduce steric hindrance between each one or more identified peptides of the instant disclosure, is well translated, and supports or allows processing of the each one or more identified polypeptides of the instant disclosure. In aspects, the linker should have little or no immunogenic sequence elements. In aspects, each peptide or polypeptide of the concatemeric polypeptide may optionally have one or more linkers, which may optionally be cleavage sensitive sites, adjacent to their N and/or C terminal end. In such a concatemeric peptide, two or more of the peptides may have a cleavage sensitive site between them. Alternatively two or more of the peptides may be connected directly to one another or through a linker that is not a cleavage sensitive site.

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier, or diluent” or the like refer to an excipient, carrier, or diluent that can be administered to a subject, together with an agent, and which does not destroy the pharmacological activity thereof and is nontoxic when administered in doses sufficient to deliver a therapeutic amount of the agent.

As used herein, “GAA” refers to an enzyme acid alpha glucosidase or lysosomal alpha glucosidase (LYAG), including naturally expressed and recombinant forms thereof. In some instances, GAA may include a recombinant human GAA, rhGAA, that may be utilized in enzyme replacement therapy. In some aspects, GAA may include the amino acid sequence set forth in SEQ ID NO: 101, as well as variants thereof, including one or more of S46P, C103G, C103R, C108G, C127F, R190H, Y191C, L208P, P217L, G219RR224P, R224Q, R224W, T234K, T234R, A237V, S251 L, S254L, E262K, P266S, P285R, P285S, L291F, L291P, Y292C, G293R, L299R, H308L, H308P, G309R, L312R, N316I, M318K, M318T, P324L, W330G, G335E, G335R, P347R, L355P, P361L, C374R, R375L, G377R, P397L, Q401R, W402R, D404N, L405P, M408V, D419V, 431-433 missing, R437C, R437H, A445P, Y455F, PP457H, P457L, 459 missing, G478R, W481R, P482R, G483V, A486P, D489N, M519T, M519V, E521K, E521Q, P522A, P522S, S523Y, F525Y, S529V, P545L, G549R, L552P, 1557F, C558S, S566P, H568L, N570K, H572Q, Y575C, Y575S, G576R, E579K, R585M, R594H, R594P, S599Y, R600C, R600H, S601L, T602A, 607-612 missing, G607D, A610P, H612Y, H612Y, T614K, G615R, S619R, S627P, N635K, G638V, G638W, L641V, G643R, D645E, D645H, C647W, G648D, G648S, R660H, R672Q, R672T, R672W, 675 missing, R702C, R702L, L705P, R725W, T737N, Q743K, W746G, W746S, Y766C, P768R, R819P, A880D, L901Q, 903 missing, P913R, V916F, N925NGVPVSN, L935P, V949D and combinations thereof.

As used herein, a “free thiol” refers to a thiol side chain of an amino acid optionally in a polypeptide and/or protein, wherein the thiol contains a sulfhydryl group. For example, free thiols are not bound to the side chains of other amino acids through intramolecular or intermolecular disulfide bonds.

As used herein, “functionalities” are groups on blood components, including mobile and fixed proteins, to which reactive groups on modified therapeutic peptides react to form covalent bonds. Functionalities may include hydroxyl groups for bonding to ester reactive groups, thiol groups for bonding to maleimides, imidates and thioester groups; amino groups for bonding to activated carboxyl, phosphoryl or any other acyl groups on reactive groups.

As used herein, “blood components” may be either fixed or mobile. Fixed blood components are non-mobile blood components and include tissues, membrane receptors, interstitial proteins, fibrin proteins, collagens, platelets, endothelial cells, epithelial cells and their associated membrane and membranous receptors, somatic body cells, skeletal and smooth muscle cells, neuronal components, osteocytes and osteoclasts and all body tissues especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components may be at least about 12 hours.

As used herein, the term “purpose built computer program” refers to a computer program designed to fulfill a specific purpose; typically to analyze a specific set of raw data and answer a specific scientific question.

As used herein, the term “z-score” indicates how many standard deviations an element is from the mean. A z-score can be calculated from the following formula. z=(X−μ)/σ where z is the z-score, X is the value of the element, p is the population mean, and a is the standard deviation.

As used herein, the singular forms “a,” “an,” and “the” are intended to include the plural forms, including “at least one,” unless the content clearly indicates otherwise. “Or” means “and/or.” As used herein, the term “and/or” and “one or more” includes any and all combinations of the associated listed items. For example, the term “one or more” with respect to the “one or more of SEQ ID NOS: 1-73 of the present disclosure” includes any and all combinations of SEQ ID NOS: 1-73. The term “or a combination thereof” means a combination including at least one of the foregoing elements.

The following abbreviations and/or acronyms are used throughout this application:
APC antigen presenting cells
CEF cytomegalovirus, Epstein-Barr virus and influenza virus
CFSE dye carboxyfluorescein succinimidyl ester dye
DMSO dimethyl sulfoxide
DR antibody antigen D related antibody
ELISA enzyme-linked immunosorbent assay
FACS fluorescence-activated cell sortings
Fmoc 9-fluoronyl methoxy carbonyl
FV human coagulation Factor V
FVIII human coagulation Factor VIII
HLA human leukocyte antigen
HPLC high-performance liquid chromatography
IVIG intravenous purified Immunoglobulin G antibody
MFI mean fluorescence index
MHC major histocompatibility complex
PBMC peripheral blood mononuclear cell
PI proliferation index
RPMI Roswell Park Memorial Institute medium
T_{eff} effector T cell
T_{Reg} regulatory T cell
TT tetanus toxoid
UV ultraviolet

As used herein, a “variant” peptide or polypeptide (including a variant Tregitope) can differ in amino acid sequence by one or more substitutions, deletions, insertions, inversions, fusions, and truncations or a combination of any of these. In aspects, a variant peptide or polypeptide (including a variant T-cell epitope) can differ in amino acid sequence by one or more substitutions, deletions, insertions, inversions, fusions, and truncations or a combination of any of these provided said variants retain MHC binding propensity and/or TCR specificity, and/or regulatory T cell stimulating or suppressive activity.

As used herein, an “antibody” can take various forms, including, but not limited to, one or more of the following: monoclonal or polyclonal; mouse, human, or humanized; monospecific or bispecific; glycosylated; Fc-modified; antibody-drug conjugate; antibody of different class or subclass, such as IgG (e.g., IgG1, IgG2, IgG3, or IgG4), IgM, IgE, or IgA; and/or antibody fragments or derivatives thereof (e.g., Fab, scFv, diabody, sdAb, or tandem sccFv.

The present disclosure also includes polypeptide fragments of the Tregitopes of the invention. The present disclosure also encompasses fragments of the variants of the Tregitopes described herein, provided said fragments and/or variants retain MHC binding propensity and/or TCR specificity and/or regulatory T cell stimulating or suppressive activity.

The present disclosure also provides chimeric or fusion polypeptides (which in aspects may be isolated, synthetic, or recombinant) wherein one or more of the instantly-disclosed Tregitopes is a part thereof. In aspects, a chimeric or fusion polypeptide composition comprises one or more polypeptides (Treg activating regulatory T-cell epitope, Tregitope, Tregitope peptide, or T-cell epitope polypeptide) of the instant disclosure linked to a heterologous polypeptide (e.g. but not limited to, IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody)). As previously stated, the term “heterologous polypeptide” is intended to mean that the one or more Tregitope (e.g., one or more of SEQ ID NOS: 1-73) are heterologous to, or not included naturally, in the heterologous polypeptide. In aspects, the one or more Tregitope may be inserted into the heterologous polypeptide (e.g., through mutagenesis techniques or other known means in the art), may be added to the C-terminus, and/or added to the N-terminus of the heterologous polypeptide. In aspects, one or more Tregitopes of the instant disclosure may also be fused to or inserted internally within (e.g., but not limited to, site directed mutagenesis or other recombinant techniques) a human GAA sequence (SEQ ID NO: 101) or a variant thereof, such as in instances where the Tregitope is not located in its natural position within the human GAA sequence or where the subjects own GAA is missing such a Tregitope (e.g., if a particular patient has a mutated or missing corresponding section in their own expressed GAA)). In aspects, the one or more Tregitopes may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, chimeric or fusion polypeptides comprise one or more Tregitope of the present disclosure operatively linked to a heterologous polypeptide. “Operatively linked” indicates that the polypeptide (e.g., the one or more Treg activating regulatory T-cell epitope, Tregitope, Tregitope peptide, or T-cell epitope polypeptide of the present disclosure) and the heterologous protein are fused in-frame or chemically-linked or otherwise bound. For example, in aspects, the one or more Tregitopes may be covalently bound to one or more internal conjugation site(s) in a Fc domain as disclosed in U.S. Pat. Nos. 8,008,453, 9,114,175, and/or 10,188,740 (each of which are herein incorporated by reference in their entirety). In aspects, a chimeric or fusion polypeptide composition comprises a polypeptide, said polypeptide having a sequence comprising one or more of SEQ ID NOS: 1-73 of the present disclosure, wherein said one or more of SEQ ID NOS: 1-73 is not naturally included in the polypeptide and/or said of one or more of SEQ ID NOS: 1-73 is not located at its natural position in the polypeptide. In aspects, the one or more of SEQ ID NOS: 1-73 of the present disclosure can be joined, linked to (e.g., fused in-frame, chemically-linked, or otherwise bound), and/or inserted into the polypeptide. In aspects, the one or more of SEQ ID NOS: 1-73 of the present disclosure can be joined or linked to (e.g., fused in-frame, chemically-linked, or otherwise bound) to a small molecule, drug, or drug fragment, for example but not limited to, a drug or drug fragment that is binds with high affinity to defined HLAs. In aspects of the above chimeric, fusion polypeptide, and fusion product compositions, the one or more polypeptides (Treg activating regulatory T-cell epitope, Tregitope, Tregitope peptide, or T-cell epitope polypeptide) of the present disclosure comprises, consists of, or consists essentially of one or more of SEQ ID NOS: 1-73. In aspects, the one or more polypeptides (Treg activating regulatory T-cell epitope, Tregitope, Tregitope peptide, or T-cell epitope polypeptide) of the present disclosure comprises, consists of, or consists essentially of a sequence one or more of SEQ ID NOS: 1-6.

An “isolated” polypeptide (e.g., an isolated Treg activating regulatory T-cell epitope, Tregitope, Tregitope peptide, or T-cell epitope polypeptide) can be purified from cells that naturally express it, purified from cells that have been altered to express it (recombinant), or synthesized using known protein synthesis methods. In one embodiment, a Tregitope is produced by recombinant DNA or RNA techniques. For example, a nucleic acid molecule encoding the Tregitope is cloned into an expression vector, the expression vector introduced into a host cell and the polypeptide expressed in the host cell. The Tregitope can then be isolated from the cells by an appropriate purification scheme using standard protein purification techniques.

For the purposes of the present disclosure, peptides, polypeptides, concatemeric peptides, or chimeric or fusion polypeptides of the instant disclosure can include, for example, modified forms of naturally occurring amino acids such as D-stereoisomers, non-naturally occurring amino acids; amino acid analogs; and mimetics. Further, in aspects, peptides, polypeptides, concatemeric peptides, or chimeric or fusion polypeptides of the instant disclosure can include retro-inverso peptides of the instantly disclosed peptides, polypeptides, concatemeric peptides, or chimeric or fusion polypeptides of the instant disclosure, provided said retro-inverso peptides, polypeptides, concatemeric peptides, or chimeric or fusion polypeptides of the instant disclosure at least in part retain MHC binding propensity and/or TCR specificity.

Although any methods and materials similar or equivalent to those described herein can be used in the practice or testing of the present disclosure, the preferred methods and materials are described. Other features, objects, and advantages of the present disclosure will be apparent from the description and the Claims. In the Specification and the appended Claims, the singular forms include plural referents unless the context clearly dictates otherwise. Unless defined otherwise, all technical and scientific terms used herein have the same meaning as commonly understood by one of ordinary skill in the art to which this invention belongs. All references cited herein are incorporated herein by reference in their entirety and for all purposes to the same extent as if each individual publication, patent, or patent application was specifically and individually indicated to be incorporated by reference in its entirety for all purposes.

### Polypeptides, Concatemeric Polypeptides, and Chimeric or Fusion Polypeptides

In aspects, the present disclosure provides Tregitope compounds and compositions, including polypeptides (Treg activating regulatory T-cell epitope, Tregitope, Tregitope peptide, or T-cell epitope polypeptide, which in aspects may be isolated, synthetic, or recombinant) as disclosed herein; nucleic acids, expression cassettes, plasmids, expression vectors, recombinant viruses, or cells (all of which in aspects may be isolated, synthetic, or recombinant) as disclosed herein; isolated, synthetic, or recombinant chimeric or fusion polypeptide compositions as disclosed herein; and/or pharmaceutical compositions or formulations as disclosed herein. In aspects, the Tregitope compounds and compositions include one or more of the regulatory Tregitopes of Tables 1-8 (including fragments thereof, variants thereof, and fragments of such variants thereof, provided said fragments and/or variants retain MHC binding propensity and/or TCR specificity, and/or regulatory T cell stimulating or suppressive activity). In certain aspects, the Tregitopes can be capped with an n-terminal acetyl and/or c-terminal amino group.

In one aspect, the present disclosure provides a novel class of T cell epitopes (which may be isolated, synthetic, or recombinant), termed ‘Tregitopes’, which comprise a peptide or polypeptide chain derived from common human proteins. Tregitopes of the present disclosure are highly conserved among known variants of their source proteins (e.g., present in more than 10% of known variants). Tregitopes of the present disclosure comprise at least one putative T cell epitope as identified by EpiMatrix™ analysis. EpiMatrix™ is a proprietary computer algorithm developed by EpiVax (Providence, Rhode Island), which is used to screen protein sequences for the presence of putative T cell epitopes. Input sequences are parsed into overlapping 9-mer frames where each frame overlaps the last by 8 amino acids. Each of the resulting frames is then scored for predicted binding affinity with respect to a panel of eight common Class II HLA alleles (DRB1*0101, DRB1*0301, DRB1*0401, DRB1*0701, DRB1*0801, DRB1*1101, DRB1*1301, and DRB1*1501). Raw scores are normalized against the scores of a large sample of randomly generated peptides. The resulting “Z” score is reported. In aspects, any 9-mer peptide with an allele-specific EpiMatrix™ Z-score in excess of 1.64, theoretically the top 5% of any given sample, is considered a putative T cell epitope.

Peptides containing clusters of putative T cell epitopes are more likely to test positive in validating in vitro and in vivo assays. The results of the initial EpiMatrix™ analysis are further screened for the presence of putative T cell epitope “clusters” using a second proprietary algorithm known as Clustimer™ algorithm. The Clustimer™ algorithm identifies sub-regions contained within any given amino acid sequence that contains a statistically unusually high number of putative T cell epitopes. Typical T-cell epitope “clusters” range from about 9 to roughly 30 amino acids in length and, considering their affinity to multiple alleles and across multiple 9-mer frames, can contain anywhere from about 4 to about 40 putative T cell epitopes. Each epitope cluster identified an aggregate EpiMatrix™ score is calculated by summing the scores of the putative T cell epitopes and subtracting a correcting factor based on the length of the candidate epitope cluster and the expected score of a randomly generated cluster of the same length. EpiMatrix™ cluster scores in excess of +10 are considered significant. In aspects, the Tregitopes of the instant disclosure contain several putative T cell epitopes forming a pattern known as a T cell epitope cluster.

Many of the most reactive T cell epitope clusters contain a feature referred to as an “EpiBar™”. As previously described, an EpiBar™ is a single 9-mer frame that is predicted to be reactive to at least four different HLA alleles. In aspects, the Tregitopes of the present disclosure can comprise one or more EpiBars™.

The JanusMatrix system (EpiVax, Providence, Rhode Island) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and as such may be very useful Tregitopes of the present disclosure.

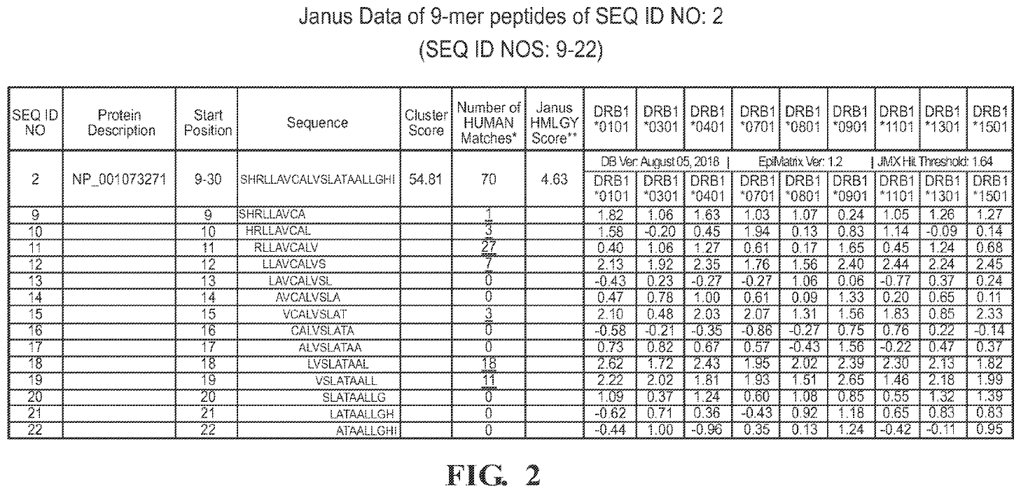

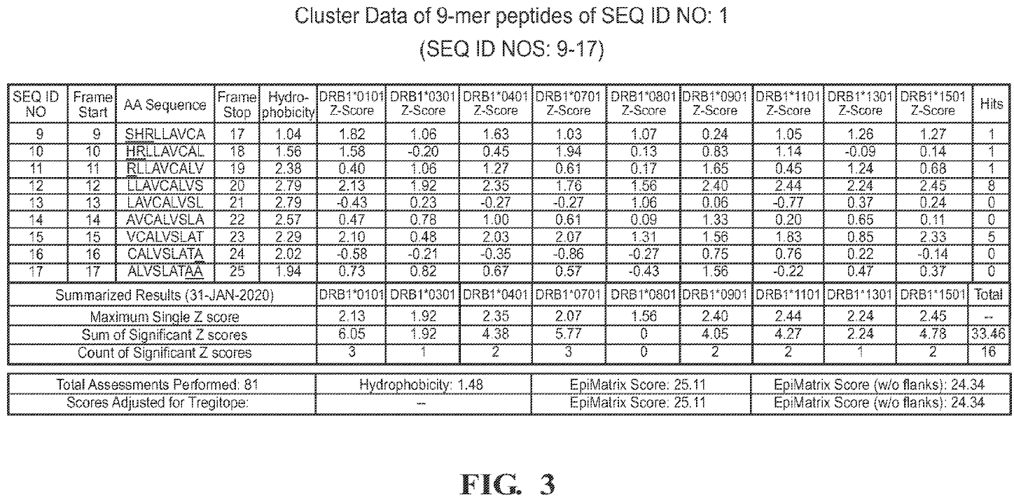

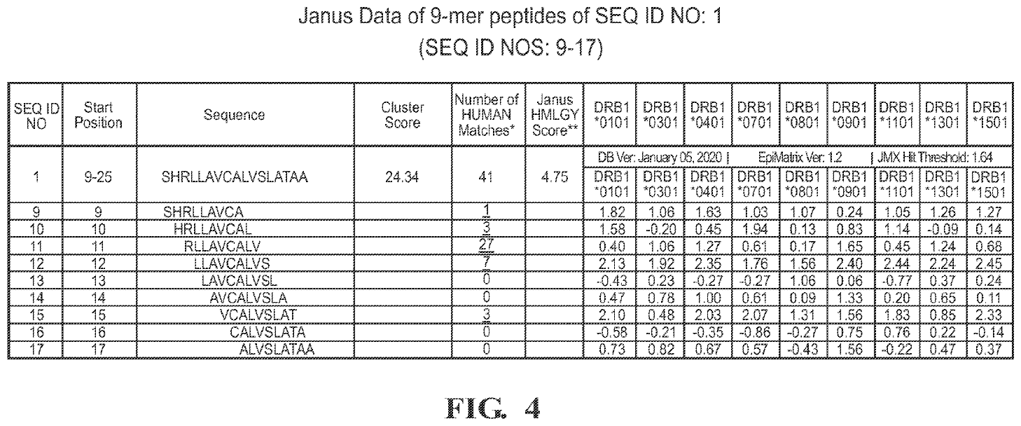

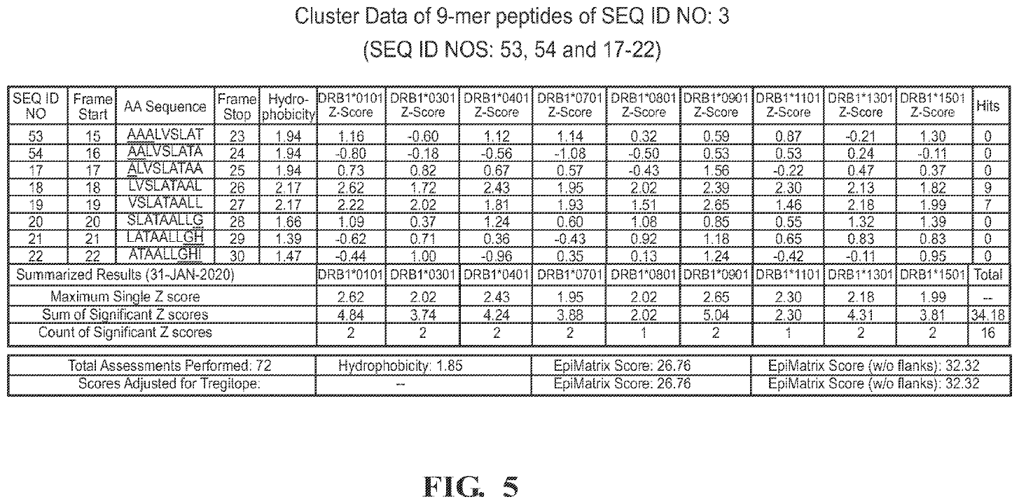

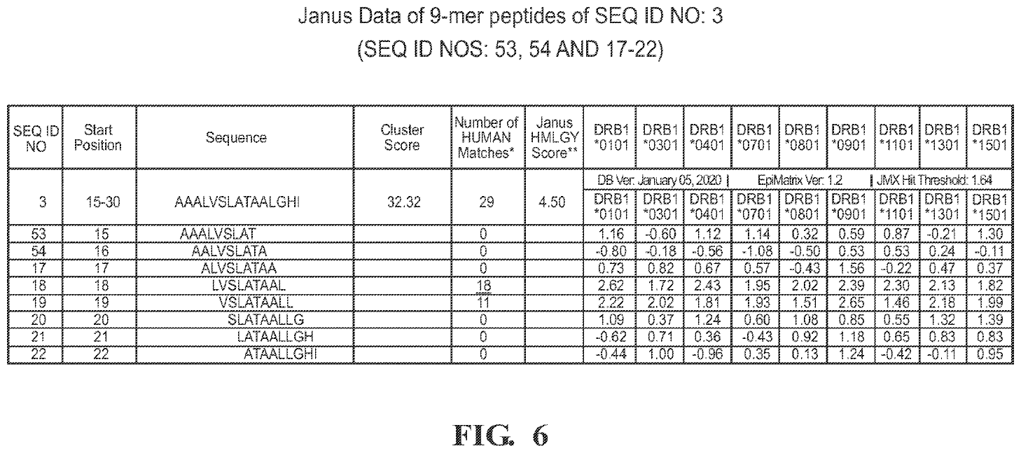

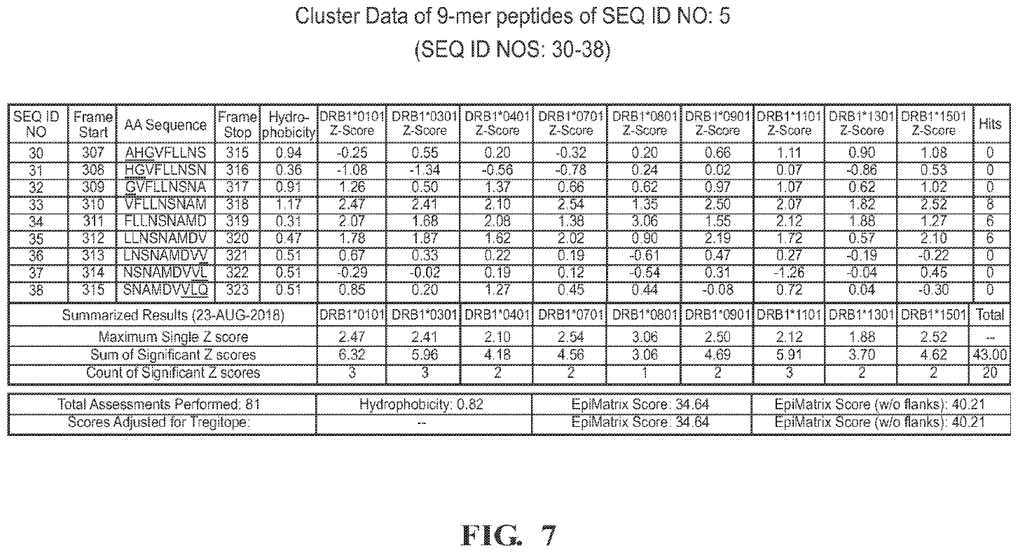

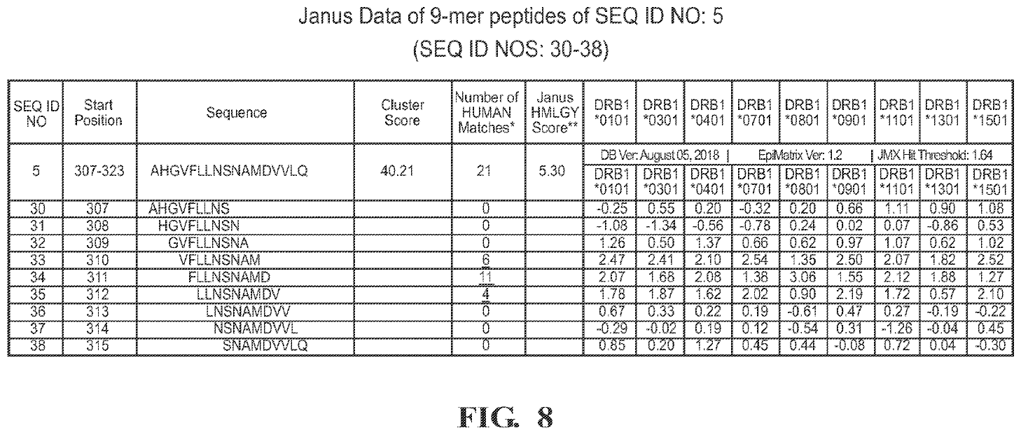

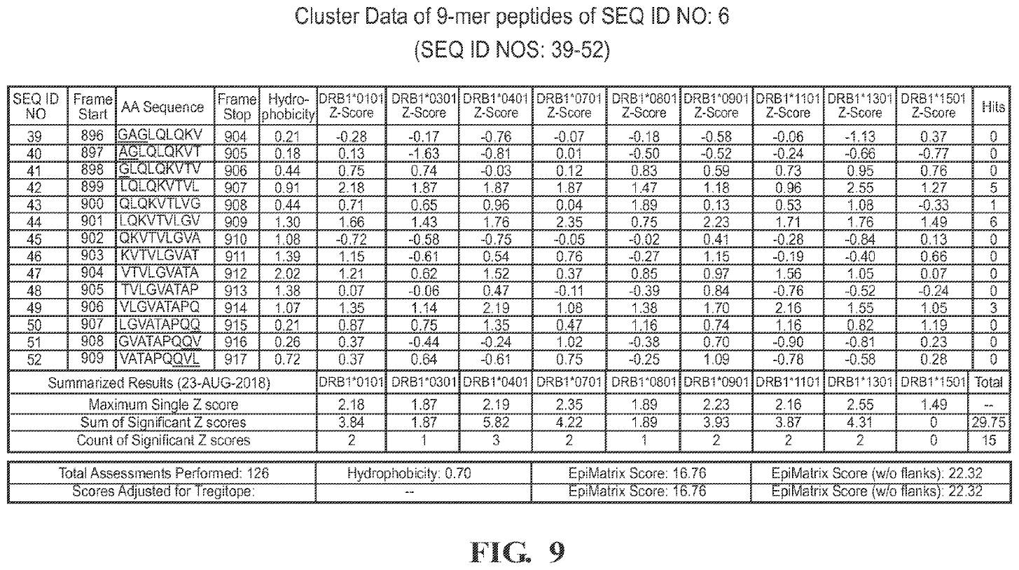

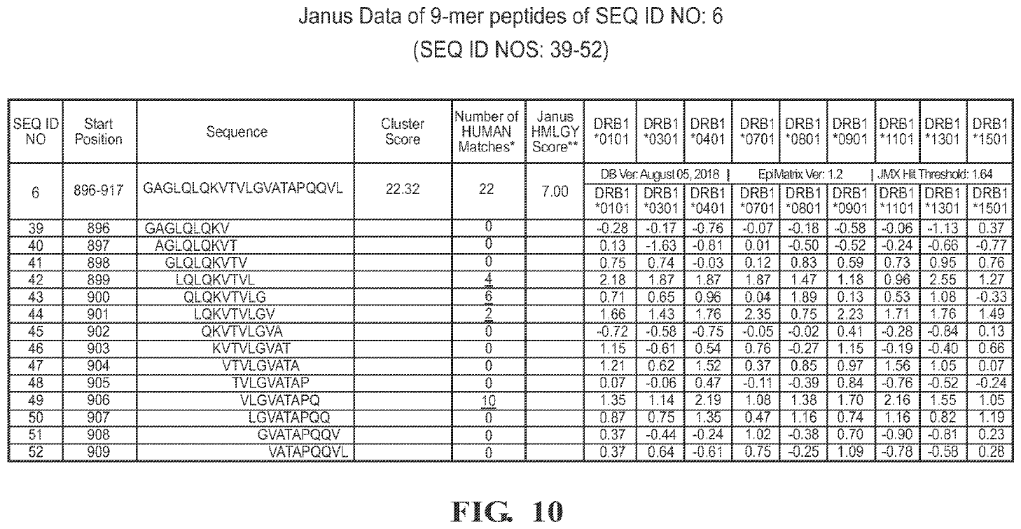

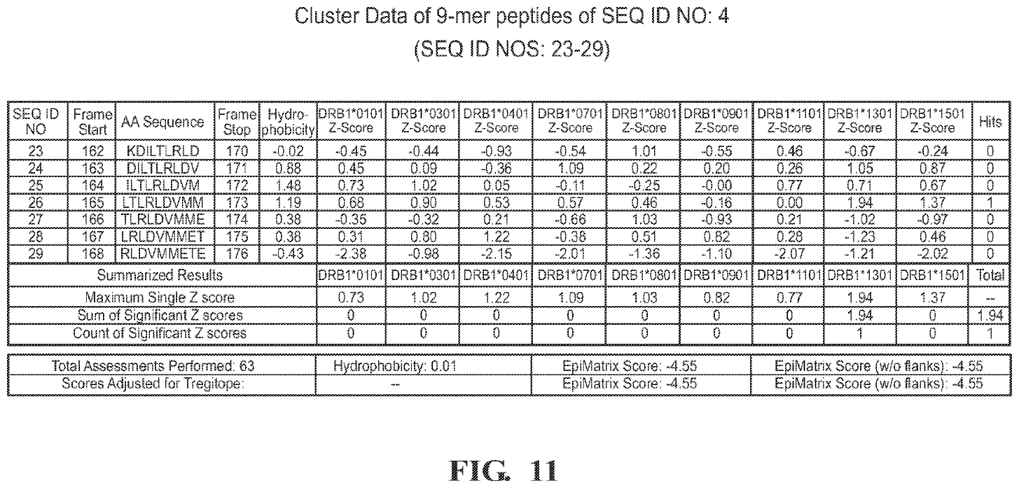

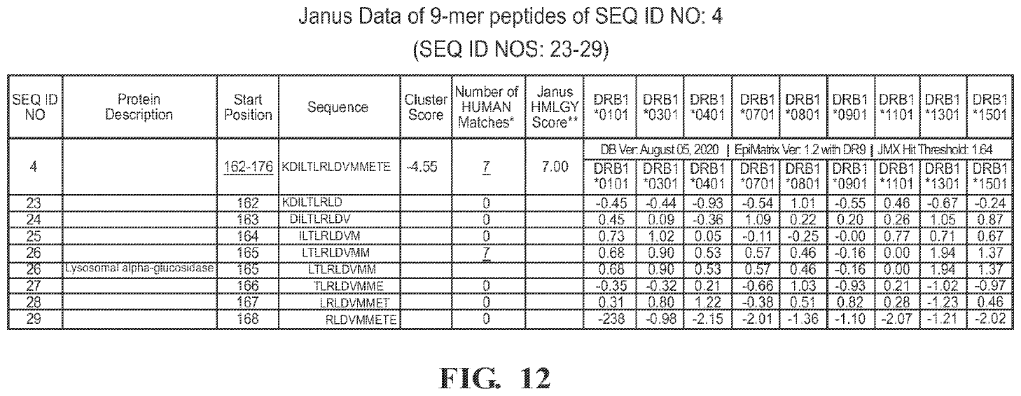

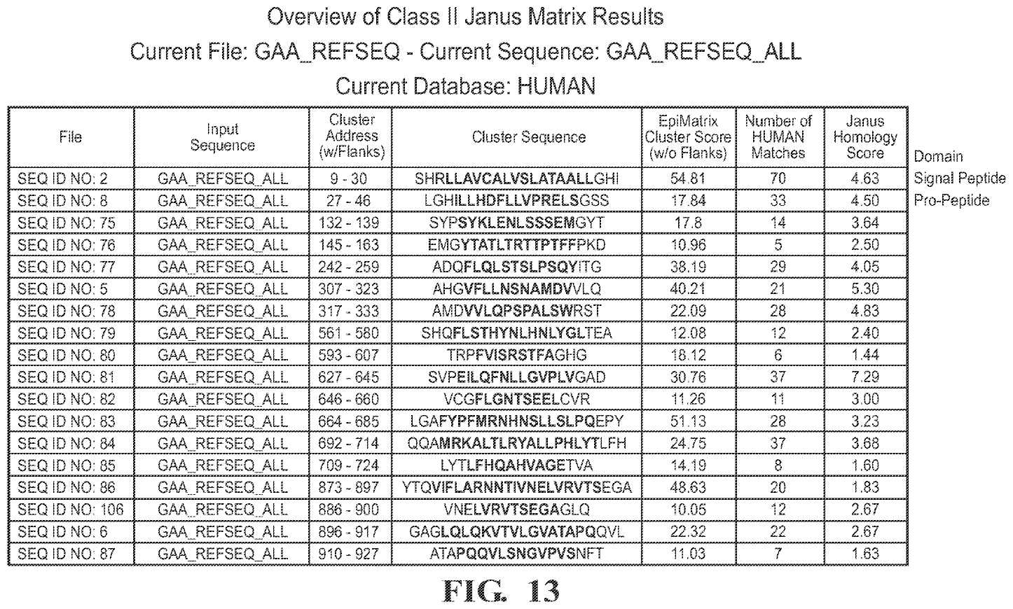

FIG. 13 is the overview of JanusMatrix results for identified Tregitopes of the instant disclosure. FIG. 1 is the Cluster report and FIG. 2 is the JanusMatrix report for the Tregitope of SEQ ID NO: 2 and the 9-mers contained within SEQ ID NO: 2, including SEQ ID NOS: 9-17. FIG. 3 is the Cluster report and FIG. 4 is the JanusMatrix report for the Tregitope of SEQ ID NO: 1 and the 9-mers contained within SEQ ID NO: 1, including SEQ ID NOS: 9-22. FIG. 5 is the Cluster report and FIG. 6 is the JanusMatrix report for the Tregitope of SEQ ID NO: 3 and the 9-mers contained within SEQ ID NO: 3, including SEQ ID NOS: 9-17-22, 53 and 54. FIG. 7 is the Cluster report and FIG. 8 is the JanusMatrix report for the Tregitope of SEQ ID NO: 5 and the 9-mers contained within SEQ ID NO: 5, including SEQ ID NOS: 30-38. FIG. 9 is the Cluster report and FIG. 10 is the JanusMatrix report for the Tregitope of SEQ ID NO: 6 and the 9-mers contained within SEQ ID NO: 6, including SEQ ID NOS: 39-52. FIG. 11 is the Cluster report and FIG. 12 is the JanusMatrix report for the Tregitope of SEQ ID NO: 4 and the 9-mers contained within SEQ ID NO: 4, including SEQ ID NOS: 23-29. FIG. 12 also depicts several other genes with homology to the sequence set forth in SEQ ID NO: 26. The additional identified peptides are matches from the human proteome identified with the Janus algorithm as sharing a TCR binding face, indicating a potential for tolerance to SEQ ID NO: 26. For each of FIGS. 2, 4, 6, 8, 10 and 12 * is the count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. Further, the Janus Homology Score** represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

In aspects, Tregitopes of the present disclosure bind to at least one and preferably two or more common HLA class II molecules with at least a moderate affinity (e.g., in aspects, <1000 μM IC₅₀, <500 μM IC₅₀, <400 μM IC₅₀, <300 μM IC₅₀, or <200 μM IC₅₀ in HLA binding assays based on soluble HLA molecules). In aspects, Tregitopes of the present disclosure are capable of being presented at the cell surface by APCs in the context of at least one and, in other aspects, two or more alleles of the HLA. In this context, the Tregitope-HLA complex can be recognized by naturally occurring T_{Regs} (in aspects, including natural T_{Regs} and/or adaptive T_{Regs}) having TCRs that are specific for the Tregitope-HLA complex and circulating in normal control subjects. In aspects, the recognition of the Tregitope-HLA complex can cause the matching regulatory T cell to be activated and to secrete regulatory cytokines and chemokines.

In aspects, the present disclosure is directed to a polypeptide (which may be termed herein as “Treg activating regulatory T-cell epitope”, “Tregitope”, “Tregitope peptide”, or “T-cell epitope polypeptide”) having a sequence comprising, consisting of, or consisting essentially of one or more of SEQ ID NOS: 1-73 (and fragments and variants thereof). The phrase “consisting essentially of” is intended to mean that a polypeptide according to the present disclosure, in addition to having the sequence according to any of SEQ ID NOS: 1-73 or a variant thereof, contains additional amino acids or residues that may be present at either terminus of the peptide and/or on a side chain that are not necessarily forming part of the peptide that functions as an MHC ligand and provided they do not substantially impair the activity of the peptide to function as a Tregitope. In aspects, a polypeptide (Treg activating regulatory T-cell epitope, Tregitope, Tregitope peptide, or T-cell epitope polypeptide) comprises, consists of, or consists essentially of one or more of SEQ ID NOS: 1-6. In certain aspects, such polypeptides can be capped with an n-terminal acetyl and/or c-terminal amino group.

In aspects, the instant disclosure is directed to a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73. In aspects, the instant disclosure is directed to a peptide or polypeptide have a core amino acid sequence comprising, consisting of, or consisting essentially of one or more peptides or polypeptides having an amino acid sequence of SEQ ID NOS: 1-73, and optionally having extensions of 1 to 12 amino acids on the C-terminal and/or the N-terminal of the core amino acid sequence, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio). In aspects, the instant disclosure is directed to a peptide or polypeptide having a core sequence comprising, consisting of, or consisting essentially of one or more peptides or polypeptides having an amino acid sequence of SEQ ID NOS: 1-73 (and/or fragments and variants thereof), optionally with extensions of 1 to 12 amino acids on the C-terminal and/or the N-terminal, wherein the overall number of these flanking amino acids is 1 to 12, 1 to 3, 2 to 4, 3 to 6, 1 to 10, 1 to 8, 1 to 6, 2 to 12, 2 to 10, 2 to 8, 2 to 6, 3 to 12, 3 to 10, 3 to 8, 3 to 6, 4 to 12, 4 to 10, 4 to 8, 4 to 6, 5 to 12, 5 to 10, 5 to 8, 5 to 6, 6 to 12, 6 to 10, 6 to 8, 7 to 12, 7 to 10, 7 to 8, 8 to 12, 8 to 10, 9 to 12, 9 to 10, or 10 to 12, wherein the flanking amino acids can be distributed in any ratio to the C-terminus and the N-terminus (for example all flanking amino acids can be added to one terminus, or the amino acids can be added equally to both termini or in any other ratio), provided that the polypeptide with the flanking amino acids is still able to bind to the same HLA molecule (i.e., retain MHC binding propensity) as said polypeptide core sequence without said flanking amino acids. In aspects, said polypeptide with the flanking amino acids is still able to bind to the same HLA molecule (i.e., retain MHC binding propensity) and/or retain the same TCR specificity, and/or retain regulatory T cell stimulating or suppressive activity, as said polypeptide core sequence without said flanking amino acids. In aspects, said flanking amino acid sequences are those that also flank the peptides or polypeptides included therein in the naturally occurring protein (e.g., as found in GAA/LYAG, e.g., SEQ ID NO: 101 as well as variants thereof, including variants having one or more of S46P, C103G, C103R, C108G, C127F, R190H, Y191C, L208P, P217L, G219RR224P, R224Q, R224W, T234K, T234R, A237V, S251L, S254L, E262K, P266S, P285R, P285S, L291F, L291P, Y292C, G293R, L299R, H308L, H308P, G309R, L312R, N316I, M318K, M318T, P324L, W330G, G335E, G335R, P347R, L355P, P361 L, C374R, R375L, G377R, P397L, Q401R, W402R, D404N, L405P, M408V, D419V, 431-433 missing, R437C, R437H, A445P, Y455F, PP457H, P457L, 459 missing, G478R, W481R, P482R, G483V, A486P, D489N, M519T, M519V, E521K, E521Q, P522A, P522S, S523Y, F525Y, S529V, P545L, G549R, L552P, 1557F, C558S, S566P, H568L, N570K, H572Q, Y575C, Y575S, G576R, E579K, R585M, R594H, R594P, S599Y, R6000, R600H, S601L, T602A, 607-612 missing, G607D, A610P, H612Y, H612Y, T614K, G615R, S619R, S627P, N635K, G638V, G638W, L641V, G643R, D645E, D645H, C647W, G648D, G648S, R660H, R672Q, R672T, R672W, 675 missing, R702C, R702L, L705P, R725W, T737N, Q743K, W746G, W746S, Y766C, P768R, R819P, A880D, L901Q, 903 missing, P913R, V916F, N925NGVPVSN, L935P, V949D and combinations thereof). In aspects, said flanking amino acid sequences as described herein may serve as a MHC stabilizing region. The use of a longer peptide may allow endogenous processing by patient cells and may lead to more effective antigen presentation and induction of T cell responses. In aspects, the peptides or polypeptides can be either in neutral (uncharged) or salt forms, and may be either free of or include modifications such as glycosylation, side chain oxidation, or phosphorylation. In certain aspects, the peptides or polypeptides can be capped with an n-terminal acetyl and/or c-terminal amino group.

In aspects, the instant disclosure is directed to a polypeptide comprising an amino acid sequence having at least 75%, 80%, 85%, 90%, or 95% homology to any one of SEQ ID NOS: 11-73 (and/or fragments thereof), wherein said polypeptide is still able to bind to a same HLA molecule (i.e., retain MHC binding propensity) and/or retain the same TCR specificity, and/or retain regulatory T cell stimulating or suppressive activity.

In aspects, the present disclosure is directed to a concatemeric polypeptide or peptide that comprises at one or more of the instantly-disclosed polypeptides or peptides (e.g., but not limited to, a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) linked, fused, or joined together (e.g., fused in-frame, chemically-linked, or otherwise bound) to an additional peptide or polypeptide. Such additional peptide or polypeptide may be one or more of the instantly instantly-disclosed polypeptides or peptides, or may be an additional peptide or polypeptide of interest. In aspects a concatemeric peptide is composed of 1 or more, 2 or more, 3 or more, 4 or more, 5 or more 6 or more 7 or more, 8 or more, 9 or more of the instantly-disclosed peptides or polypeptides. In other aspects, the concatemeric peptides or polypeptides include 1000 or more, 1000 or less, 900 or less, 500 or less, 100 or less, 75 or less, 50 or less, 40 or less, 30 or less, 20 or less or 100 or less peptide epitopes. In yet other embodiments, a concatemeric peptide has 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 of the instantly-disclosed peptides or polypeptides linked, fused, or joined together. Each peptide or polypeptide of the concatemeric polypeptide may optionally have one or more linkers, which may optionally be cleavage sensitive sites, adjacent to their N and/or C terminal end. Such suitable linkers and cleavage sensitive sites, including AAY cleavage motifs or a poly GS linker which may be include on the N terminus of the C-terminal element, are known in the art. In such a concatemeric peptide, two or more of the peptide epitopes may have a cleavage sensitive site between them. Alternatively, two or more of the peptide epitopes may be connected directly to one another or through a linker that is not a cleavage sensitive site. In aspects, such linker is antigenically neutral, and the liker is preferably less than the length of a peptidyl backbone of 9 amino acids linearly arranged. In aspects, linker length is the length of a peptidyl backbone of between 2 and 8 amino acids, linearly arranged. In aspects, the spacer is unable to hydrogen bond in any spatially distinct manner to other distinct elements of the enhancing hybrid peptide.

In aspects, and with respect to antigenically neutral linker elements, various chemical groups may be incorporated as linkers instead of amino acids. Examples are described in U.S. Pat. No. 5,910,300, the contents of which are incorporated herein by reference. In aspects, a linker may be comprised of an aliphatic chain optimally interrupted by heteroatoms, for example a C2-C6 alkylene, or ═N—(CH2)2-6-N═. Alternatively, a spacer may be composed of alternating units, for example of hydrophobic, lipophilic, aliphatic and aryl-aliphatic sequences, optionally interrupted by heteroatoms such as O, N, or S. Such components of a spacer are preferably chosen from the following classes of compounds: sterols, alkyl alcohols, polyglycerides with varying alkyl functions, alkyl-phenols, alkyl-amines, amides, hydroxyphobic polyoxyalkylenes, and the like. Other examples are hydrophobic polyanhydrides, polyorthoesters, polyphosphazenes, polyhydroxy acids, polycaprolactones, polylactic, polyglycolic polyhydroxy-butyric acids. A linker may also contain repeating short aliphatic chains, such as polypropylene, isopropylene, butylene, isobutylene, pentamethylene, and the like, separated by oxygen atoms.

Additional peptidyl sequences which can be used in as possible linkers are described in U.S. Pat. No. 5,856,456, the contents of which are incorporated herein by reference. In one embodiment, a linker has a chemical group incorporated within which is subject to cleavage. Without limitation, such a chemical group may be designed for cleavage catalyzed by a protease, by a chemical group, or by a catalytic monoclonal antibody. In the case of a protease-sensitive chemical group, tryptic targets (two amino acids with cationic side chains), chymotryptic targets (with a hydrophobic side chain), and cathepsin sensitivity (B, D or S) are favored. The term ‘tryptic target’ is used herein to describe sequences of amino acids which are recognized by trypsin and trypsin-like enzymes. The term ‘chymotryptic target’ is used herein to describe sequences of amino acids which are recognized by chymotrypsin and chymotrypsin-like enzymes. In addition, chemical targets of catalytic monoclonal antibodies, and other chemically cleaved groups are well known to persons skilled in the art of peptide synthesis, enzymatic catalysis, and organic chemistry in general, and can be designed into the hybrid structure and synthesized, using routine experimental methods.

In aspects, a concatemeric polypeptide of the instant disclosure is produced using the EpiAssembler System (EpiVax). The EpiAssembler system is useful for assembling overlapping epitopes to Immunogenic Consensus Sequences (ICS). EpiAssembler is an algorithm that optimizes the balance between pathogen and population coverage. EpiAssembler uses the information from the sequences produced by Conservatrix and EpiMatrix to form highly immunogenic consensus sequences.

In aspects of above-described concatemeric peptides or polypeptides, the concatemeric peptides or polypeptides may be isolated, synthetic, or recombinant. In aspects, the concatemeric peptides or polypeptides can be in either neutral (uncharged) or salt forms, and may be either free of or include modifications such as glycosylation, side chain oxidation, or phosphorylation. In certain aspects, the concatemeric polypeptides can be capped with an N-terminal acetyl and/or C-terminal amino group.

As used herein, two polypeptides (or a region of the polypeptides) are substantially homologous or identical when the amino acid sequences are at least about 45-55%, typically at least about 70-75%, more typically at least about 80-85%, more typically greater than about 90%, and more typically greater than 95% or more homologous or identical. To determine the percent homology or identity of two amino acid sequences, or of two nucleic acid sequences, the sequences are aligned for optimal comparison purposes (e.g., gaps can be introduced in the sequence of one polypeptide or nucleic acid molecule for optimal alignment with the other polypeptide or nucleic acid molecule). The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As is known in the art, the percent identity between the two sequences is a function of the number of identical positions shared by the sequences, taking into account the number of gaps, and the length of each gap, which need to be introduced for optimal alignment of the two sequences. Sequence homology for polypeptides is typically measured using sequence analysis software. As used herein, amino acid or nucleic acid “homology” is equivalent to amino acid or nucleic acid “identity”. In aspects, the percent homology between the two sequences is a function of the number of identical positions shared by the sequences (e.g., percent homology equals the number of identical positions/total number of positions ×100).

In aspects, the present disclosure also encompasses polypeptides having a lower degree of identity but having sufficient similarity so as to perform one or more of the same functions performed by a polypeptide of the instant disclosure (e.g., a polypeptide having a sequence comprising, consisting of, or consisting essentially of one or more of SEQ ID NOS: 1-73 and/or fragments and variants thereof, and optionally 1 to 12 additional amino acids distributed in any ratio on the N terminus and/or C-terminus of the polypeptide of SEQ ID NOS: 1-73; and concatemeric peptides as disclosed herein). Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, Met, and lie; interchange of the hydroxyl residues Ser and Thr, exchange of the acidic residues Asp and Glu, substitution between the amide residues Asn and Gln, exchange of the basic residues His, Lys and Arg and replacements among the aromatic residues Trp, Phe and Tyr. Guidance concerning which amino acid changes are likely to be phenotypically silent are found (Bowie J U et al., (1990), Science, 247(4948):130610, which is herein incorporated by reference in its entirety).

In aspects, a variant polypeptide can differ in amino acid sequence by one or more substitutions, deletions, insertions, inversions, fusions, and truncations or a combination of any of these. Variant polypeptides can be fully functional (e.g., retain MHC binding propensity and/or TCR specificity, and/or retain regulatory T cell stimulating or suppressive activity) or can lack function in one or more activities. Fully functional variants typically contain only conservative variation or variation in non-critical residues or in non-critical regions; in this case, typically MHC contact residues provided MHC binding is preserved. Functional variants can also contain substitution of similar amino acids that result in no change or an insignificant change in function (e.g., retain MHC binding propensity and/or TCR specificity, and/or retain regulatory T cell stimulating or suppressive activity). Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues. In aspects, a variant and/or a homologous polypeptide retains the desired regulatory T cell stimulating or suppressive activity of the instant disclosure. Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region; in this case, typically TCR contact residues. In aspects, functional variants of a polypeptide having a sequence (or a core sequence) comprising, consisting of, or consisting essentially of one or more of SEQ ID NOS: 1-73 as disclosed herein may contain one or more conservative substitutions, and in aspects one or more non-conservative substitutions, at amino acid residues which are not believed to be essential for functioning (with amino acid residues considered being essential for functioning, including, e.g., retain MHC binding propensity and/or TCR specificity, and/or retain regulatory T cell stimulating or suppressive activity) of the instantly-disclosed polypeptides. For example, in aspects, a variant polypeptide having a sequence (or a core sequence) comprising, consisting of, or consisting essentially of one or more of SEQ ID NOS: 1-73, or fragments thereof as disclosed herein, or a concatemeric peptide as disclosed herein, may contain one or more conservative substitutions (and in aspects, a nonconservative substitution) in one or more HLA contact residues, provided HLA binding is preserved. MHC binding assays are well known in the art. In aspects, such assays may include the testing of binding affinity with respect to MHC class I and class II alleles in in vitro binding assays, with such binding assays as are known in the art. Examples include, e.g., the soluble binding assays as disclosed in U.S. Pat. No. 7,884,184 or PCT/US2020/020089, both of which are herein incorporated by reference in their entireties. Additionally, in aspects, a fully functional variant polypeptide having a sequence (or a core sequence) comprising, consisting of, or consisting essentially of one or more of SEQ ID NOS: 1-73 as disclosed herein do not contain mutations at one or more critical residues or regions, such as TCR contact residues.

In aspects, the TCR-binding epitope (which can be referred to as TCR binding residues, TCR facing epitope, TCR facing residues, or TCR contacts) for a 9-mer identified epitope (which may be a 9-mer fragment of one or more of SEQ ID NOS: 1-73 as disclosed herein or a 9-mer fragment of a concatemeric peptide as disclosed herein) that bind to a MHC class II molecule are at position 2, 3, 5, 7, and 8 of the identified epitope, while the MHC-binding agretope (which can be referred to as MHC contacts, MHC facing residues, MHC-binding residues, or MHC-binding face) for a 9-mer identified epitope (which may be a 9-mer fragment of one or more of SEQ ID NOS: 1-73 as disclosed herein or a 9-mer fragment of a concatemeric peptide as disclosed herein) that bind to a MHC class II molecule are at position 1, 4, 6, and 9, both as counted from the amino terminal.

In aspects, the TCR binding epitope for a 9-mer identified epitope (which may be a 9-mer fragment of one or more of SEQ ID NOS: 1-73 or as disclosed herein or a 9-mer fragment of a concatemeric peptide) that binds to a MHC class I molecule are at position 4, 5, 6, 7, and 8 of the identified epitope, while the MHC binding agretope for a 9-mer identified epitope (which may be a 9-mer fragment of one or more of SEQ ID NOS: 1-73 as disclosed herein or a 9-mer fragment of a concatemeric peptide as disclosed herein) that bind to a MHC class I molecule are at position 1, 2, 3, and 9, both as counted from the amino terminal.

In aspects, the TCR binding epitope for a 10-mer identified epitope that bind to a MHC class I molecule are at position 4, 5, 6, 7, 8, and 9 of the identified epitope (which may be a 10-mer fragment of one or more of SEQ ID NOS: 1-73 as disclosed herein, or a 10-mer fragment of a concatemeric peptide as disclosed herein, or a 10-mer peptide containing a 9-mer of one or more of SEQ ID NOS: 1-73), while the MHC binding agretope for a 10-mer identified epitope (which may be a 10-mer fragment of one or more of SEQ ID NOS: 1-73 as disclosed herein or a 10-mer fragment of a concatemeric peptide as disclosed herein, or a 10-mer peptide containing a 9-mer of one or more of SEQ ID NOS: 1-73) that bind to a MHC class I molecule are at position 1, 2, 3, 9, and 10, both as counted from the amino terminal.

In aspects, the TCR-binding epitope for a 9-mer identified epitope (which may be a 9-mer fragment of one or more of SEQ ID NOS: 1-73 as disclosed herein or a 9-mer fragment of a concatemeric peptide as disclosed herein) that bind to a MHC class II molecule are at any combination of residues at positions 2, 3, 5, 7, and 8 (e.g., but not limited to, positions 3, 5, 7 and 8; positions 2, 5, 7, and 8; positions 2, 3, 5, and 7, etc.) of the identified epitope, while the MHC binding agretope for a 9-mer identified epitope (which may be a 9-mer fragment of one or more of SEQ ID NOS: 1-73 as disclosed herein or a 9-mer fragment of a concatemeric peptide as disclosed herein) is the complementary face to the TCR facing residues, both as counted from the amino terminal.

In aspects, the TCR binding epitope for 9-mer identified epitope (which may be a 9-mer fragment of one or more of SEQ ID NOS: 1-73 as disclosed herein or a 9-mer fragment of a concatemeric peptide as disclosed herein) that bind to a MHC class I molecule are at positions 4, 5, 6, 7, and 8; 1, 4, 5, 6, 7 and 8; or 1, 3, 4, 5, 6, 7, and 8 of the identified epitope, while the MHC binding agretope for a 9-mer identified epitope (which may be a 9-mer fragment of one or more of SEQ ID NOS: 1-73 as disclosed herein or a 9-mer fragment of a concatemeric peptide as disclosed herein) is the complementary face to the TCR facing residues, both as counted from the amino terminal.

In aspects, the TCR-binding epitope for a 10-mer identified epitope (which may be a 10-mer fragment of one or more of SEQ ID NOS: 1-73 as disclosed herein, or a 10-mer fragment of a concatemeric peptide as disclosed herein, or a 10-mer peptide containing a 9-mer of one or more of SEQ ID NOS: 1-73) that bind to a MHC class I molecule are at any combination of residues at positions 1, 3, 4, 5, 6, 7, 8, and 9 of the identified epitope, while the MHC binding agretope for a 10-mer identified epitope (which may be a 10-mer fragment of one or more of SEQ ID NOS: 1-73 as disclosed herein or a 10-mer fragment of a concatemeric peptide as disclosed herein, or a 10-mer peptide containing a 9-mer of one or more of SEQ ID NOS: 1-73) is the complementary face to the TCR facing residues, both as counted from the amino terminal.

Based on the above, it should be understood that in aspects in which one or more 9-mers and/or 10-mer epitopes are contained within a longer polypeptide and are predicted to bind one or more Class I or Class II MHC molecules and are occurring in close proximity to each other in a naturally occurring sequence (e.g., wherein position 1 of each pair of binding 9-mers and/or 10-mers fall within, e.g., 3 amino acids of each other), such epitopes may be combined to form an epitope cluster. In a given cluster, any given amino acid may be, with respect to a given 9-mer epitope or 10-mer epitope, MHC facing and, with respect to another 9-mer epitope, TCR facing.

In aspects, the present disclosure also includes fragments of the instantly-disclosed polypeptides and concatemeric polypeptides. In aspects, the present disclosure also encompasses fragments of the variants of the instantly-disclosed polypeptides and concatemeric polypeptides as described herein. In aspects, as used herein, a fragment comprises at least about nine contiguous amino acids. In aspects, the present disclosure also encompasses fragments of the variants of the T-cell epitopes described herein. Useful fragments (and fragments of the variants of the polypeptides and concatemeric polypeptides described herein) include those that retain one or more of the biological activities, particularly: MHC binding propensity and/or TCR specificity, and/or retain regulatory T cell stimulating or suppressive activity. Biologically active fragments are, for example, about 9, 10, 11, 12, 1, 14, 15, 16, 20, 25, 30, 40, 50, 60, 70, 80, 90, 100 or more amino acids in length, including any value or range therebetween. Fragments can be discrete (not fused to other amino acids or polypeptides) or can be within a larger polypeptide. Several fragments can be comprised within a single larger polypeptide. In aspects, a fragment designed for expression in a host can have heterologous pre- and pro-polypeptide regions fused to the amino terminus of the polypeptide fragment and an additional region fused to the carboxyl terminus of the fragment.

In aspects, the instantly disclosed polypeptides and concatemeric polypeptides of the present disclosure can include allelic or sequence variants (“mutants”) or analogs thereof, or can include chemical modifications (e.g., pegylation, glycosylation). In aspects, a mutant retains the same function, particularly MHC binding propensity and/or TCR specificity, and/or retain regulatory T cell stimulating or suppressive activity. In aspects, a mutant can provide for enhanced binding to MHC molecules. In aspects, a mutant can lead to enhanced binding to TCRs. In another instance, a mutant can lead to a decrease in binding to MHC molecules and/or TCRs. Also contemplated is a mutant that binds, but does not allow signaling via the TCR.

The manner of producing the polypeptides of the present disclosure will vary widely, depending upon the nature of the various elements comprising the molecule. For example, an isolated polypeptide can be purified from cells that naturally express it, purified from cells that have been altered to express it (recombinant), or synthesized using known protein synthesis methods. The synthetic procedures may be selected so as to be simple, provide for high yields, and allow for a highly purified stable product. For example, polypeptides of the instant disclosure can be produced either from a nucleic acid disclosed herein, or by the use of standard molecular biology techniques, such as recombinant techniques, mutagenesis, or other known means in the art. An isolated polypeptide can be purified from cells that naturally express it, purified from cells that have been altered to express it (recombinant), or synthesized using known protein synthesis techniques. In aspects, a polypeptide of the instant disclosure is produced by recombinant DNA or RNA techniques. In aspects, a polypeptide of the instant disclosure can be produced by expression of a recombinant nucleic acid of the instant disclosure in an appropriate host cell. For example, a nucleic acid molecule encoding the polypeptide is cloned into an expression cassette or expression vector, the expression cassette or expression vector introduced into a host cell and the polypeptide expressed in the host cell. The polypeptide can then be isolated from the cells by an appropriate purification scheme using standard protein purification techniques. Alternatively a polypeptide can be produced by a combination of ex vivo procedures, such as protease digestion and purification. Further, polypeptides of the instant disclosure can be produced using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety).

In aspects, one or more peptides or polypeptides of the instant disclosure (e.g., but not limited to, a peptide or polypeptide comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73) may be joined to, linked to (e.g., fused in-frame, chemically-linked, or otherwise bound), and/or inserted into a heterologous polypeptide. In aspects, the one or more peptides or polypeptides of the instant disclosure may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, the peptides or polypeptides can be either in neutral (uncharged) or salt forms, and may be either free of or include modifications such as glycosylation, side chain oxidation, or phosphorylation. In certain aspects, the peptides or polypeptides can be capped with an n-terminal acetyl and/or c-terminal amino group.

In aspects, a polypeptide comprises one or more of SEQ ID NOS: 1-73 of the present disclosure (in aspects, including fragments and variants thereof of SEQ ID NOS: 1-73, provided said fragments and/or variants retain MHC binding propensity and/or TCR specificity) joined to, linked to (e.g., fused in-frame, chemically-linked, or otherwise bound), and/or inserted into a heterologous polypeptide (e.g. but not limited to, an antibody (which can be IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody)). As previously described, with respect to the one or more Tregitopes of the instant disclosure, the term “heterologous polypeptide” is intended to mean that the one or more Tregitopes of the instant disclosure are heterologous to, or not included naturally, in the heterologous polypeptide. In aspects, one or more of the instantly-disclosed polypeptides (T_{reg} activating regulatory T-cell epitopes, Tregitopes, or T-cell epitope polypeptides) may be inserted into the heterologous polypeptide (e.g., through recombinant techniques, mutagenesis, or other known means in the art), may be added to the C-terminus (with or without the use of linkers, as is known in the art), and/or added to the N-terminus (with or without the use of linkers, as is known in the art) of the heterologous polypeptide. For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, one or more Tregitopes of the instant disclosure may also be fused to or inserted internally within (e.g., but not limited to, using immune engineering techniques such as but not limited to, site directed mutagenesis or other recombinant techniques) a human GAA sequence (SEQ ID NO: 101) or a variant thereof, such as in instances where the Tregitope is not located in its natural position within the human GAA sequence or where the subjects own GAA is missing such a Tregitope (e.g., if a particular patient has a mutated or missing corresponding section in their own expressed GAA)). In aspects, the one or more Tregitopes may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). In aspects, the one or more of SEQ ID NOS: 1-73 may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, the peptides or polypeptides can be either in neutral (uncharged) or salt forms, and may be either free of or include modifications such as glycosylation, side chain oxidation, or phosphorylation. In certain aspects, the peptides or polypeptides can be capped with an n-terminal acetyl and/or c-terminal amino group.

In aspects, said insertion of the one or more regulatory T cell epitopes into the heterologous polypeptide (e.g., an antibody or fragment thereof as described above, the human GAA/LYAG molecule or recombinant GAA replacement protein/supplement (or a fragments thereof) as described above, comprises insertion of all or some of the amino acids of the one or more regulatory T cell epitopes (e.g., inserting the entire sequence of the Tregitope or a fragment thereof). In aspects, said insertion of the one or more regulatory T cell epitopes into the polypeptide comprises insertion of some or all of the amino acids of the one or more regulatory T cell epitopes and removing one or more amino acids at the site of insertion of the regulatory T cell epitope amino acids. In aspects, said insertion of the one or more regulatory T cell epitopes into the polypeptide comprises mutating the sequence of the polypeptide thereof to include the one or more regulatory T cell epitopes (for example, but not limited to, introduction one or more point mutations into the polypeptide by site-directed mutagenesis or other recombinant techniques). In aspects, said insertion of the one or more regulatory T cell epitopes into the polypeptide, which in aspects will introduce the one or more regulatory T cell epitope sequences, such that the previous immunogenicity of the polypeptide sequence is decreased and the tolerogenicity of the new polypeptide sequence is enhanced. In aspects, the number of said added one or more amino acids at the site of insertion of the regulatory T cell epitope amino acids need not correspond to the number of amino acids deleted from the sequence of the polypeptide. In aspects in which the one or more regulatory T cell epitopes are inserted or fused into a particular polypeptide (e.g., a human GAA/LYAG molecule or recombinant GAA replacement protein/supplement (or a fragment thereof)) that has a mutated or missing corresponding section for which the Tregitope might be normally found, said insertion or fusion is at the site within the polypeptide where the Tregitope would normally be present. In aspects, said insertion of one or more regulatory T cell epitopes into the polypeptide thereof results in decreasing the immunogenicity of the polypeptide.

In aspects, the present disclosure is directed to polypeptide (which, in aspects, may be an isolated, synthetic, or recombinant) having a sequence comprising one or more of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73), wherein said one or more of SEQ ID NOS: 1-73 is not naturally included in the polypeptide and/or said one or more of SEQ ID NOS: 1-73 is not located at its natural position in the polypeptide. In aspects, a polypeptide (which, in aspects, may be an isolated, synthetic, or recombinant) comprises one or more of SEQ ID NOS: 1-73, wherein said polypeptide does not comprise human GAA or human LYAG or a fragment thereof. In aspects, if a polypeptide does comprise human GAA or human LYAG or a fragment thereof, then said one or more of SEQ ID NOS: 1-73 is not located in its natural position in the human GAA or LYAG or a fragment thereof. In aspects of the above-described polypeptides, the polypeptides may be isolated, synthetic, or recombinant. In aspects, the peptides or polypeptides can be either in neutral (uncharged) or salt forms, and may be either free of or include modifications such as glycosylation, side chain oxidation, or phosphorylation. In certain aspects, the peptides or polypeptides can be capped with an n-terminal acetyl and/or c-terminal amino group.

In aspects, isolated, synthetic, or recombinant Tregitopes of the present disclosure include a Tregitope of Tables 1-8 (including fragments thereof, variants thereof, and fragments of such variants thereof, provided said fragments and/or variants retain MHC binding propensity and/or TCR specificity). In aspects, isolated, synthetic, or recombinant Tregitopes of the present disclosure include one or more of:

(SEQ ID NO: 1);
SHRLLAVCALVSLATAA;
(SEQ ID NO: 2)
SHRLLAVCALVSLATAALLGHI;
(SEQ ID NO: 3)
AAALVSLATAALLGHI;
(SEQ ID NO: 4)
KDILTLRLDVMMETE;
(SEQ ID NO: 5)
AHGVFLLNSNAMDVVLQ;
(SEQ ID NO: 6)
GAGLQLQKVTVLGVATAPQQVL;
(SEQ ID NO: 12)
LLAVCALVS;
(SEQ ID NO: 15)
VCALVSLAT;
(SEQ ID NO: 18)
LVSLATAAL;
(SEQ ID NO: 19)
VSLATAALL;
(SEQ ID NO: 33)
VFLLNSNAM;
(SEQ ID NO: 34)
FLLNSNAMD;
(SEQ ID NO: 35)
LLSNNAMDV;
(SEQ ID NO: 42)
LQLQKVTVL
(SEQ ID NO: 43)
QLQKVTVLG;

and fragments thereof, variants thereof, and fragments of such variants thereof, provided said fragments and/or variants retain MHC binding propensity and/or TCR specificity. In certain aspects, such polypeptides can be capped with an n-terminal acetyl and/or c-terminal amino group.

In aspects, the Tregitope compounds and compositions of the present disclosure comprise one or more Tregitopes incorporated as an internal sequence into an Fc domain as disclosed in U.S. Pat. Nos. 7,442,778, 7,645,861, 7,655,764, 7,655,765, and/or 7,750,128 (each of which are herein incorporated by reference in their entirety). Such an internal sequence may be added by insertion (i.e., between amino acids in the previously existing Fc domain) or by replacement of amino acids in the previously existing Fc domain (i.e., removing amino acids in the previously existing Fc domain and adding peptide amino acids). In the latter case, the number of peptide amino acids added need not correspond to the number of amino acids removed from the previously existing Fc domain; for example, in aspects, the compositions may comprise an added internal sequence of 9-15 amino acids, with a sequence of 1-21 amino acids removed from the native Fc domain. In aspects, the one or more Tregitopes are inserted at or replace (e.g., full or partial replacement) one or more preferred internal sites in the Fc domain as disclosed in U.S. Pat. Nos. 7,442,778, 7,645,861, 7,655,764, 7,655,765, and/or 7,750,128.

In aspects, the Tregitope compounds and compositions of the present disclosure comprise a Tregitope peptide as described herein (e.g., but not limited to, a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) that is modified by attaching a reactive moiety to the Tregitope peptide to create a modified Tregitope peptide, wherein the reactive moiety of the modified Tregitope peptide is capable of forming a bond with a reactive functionality on a blood component, wherein upon formation of a bond between the reactive moiety of the Tregitope peptide and the reactive functionality on the blood component, a Tregitope-blood component conjugate is formed, as disclosed in U.S. Pat. Nos. 6,849,714, 6,887,470, 7,256,253, and 7,307,148 (each of which are herein incorporated by reference in their entirety). In aspects, the Tregitope in the Tregitope-blood component conjugate retains all or most of its original biologic activity. In aspects, the bond formed between the reactive moiety of the one or more modified Tregitope peptides and the blood component is a covalent bond. In aspects, the Tregitope peptide sequence is independently selected from SEQ ID NOS: 1-73, and optionally 1 to 12 additional amino acids distributed in any ratio on the N terminus and/or C-terminus of the polypeptide of SEQ ID NOS: 1-73.

Tregitope-blood component conjugates can extend the half-life of the modified polypeptides comprising Tregitopes in vivo, protect the modified polypeptides comprising Tregitopes from rapid proteolytic degradation, protect the modified polypeptides comprising Tregitopes from rapid clearance from circulation and/or rapid kidney excretion, allow for wide distribution of Tregitope-blood component conjugates throughout the body of a subject, aid in delivery of modified polypeptides comprising Tregitopes to appropriate immune cells (such as macrophages and APCs), allow the modified polypeptides comprising Tregitopes to be processed by the endocytic pathway of certain immune cells (such as macrophages and APCs), and aid in the presentation of modified polypeptides comprising Tregitopes as an antigen by said immune cells.

In aspects, the Tregitope-blood component conjugates comprise a blood component which acts as a carrier protein (e.g., albumin), and further comprise a modified polypeptide, said modified polypeptide comprising one or more regulatory T cell epitopes (termed “Tregitopes”). The modified polypeptide comprises a reactive moiety that is attached to the polypeptide, with the reactive moiety being capable of forming a bond (e.g., a covalent linkage) with a reactive functionality on the blood component. Tregitope-blood component conjugates may be formed by modifying a polypeptide comprising a Tregitope by attaching a reactive moiety to the polypeptide to create a modified polypeptide, then forming a bond between reactive moiety of the modified polypeptide with a reactive functionality on a blood component, as disclosed in U.S. Pat. Nos. 6,849,714, 6,887,470, 7,256,253, and 7,307,148, herein incorporated by reference in their entireties. In aspects of above-described Tregitope-blood component conjugates and modified polypeptides comprising Tregitopes, the Tregitope-blood component conjugates and modified polypeptides comprising Tregitopes may be isolated, synthetic, or recombinant.

In aspects, the blood components of the Tregitope-blood component conjugates may be either fixed or mobile, as disclosed in U.S. Pat. Nos. 6,849,714, 6,887,470, 7,256,253, and 7,307,148. Fixed blood components are non-mobile blood components and include tissues, membrane receptors, interstitial proteins, fibrin proteins, collagens, platelets, endothelial cells, epithelial cells and their associated membrane and membranous receptors, somatic body cells, skeletal and smooth muscle cells, neuronal components, osteocytes and osteoclasts and all body tissues, especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1 μg/ml. Mobile blood components include serum albumin, transferrin, ferritin and immunoglobulins such as IgM and IgG. The half-life of mobile blood components is at least about 12 hours. In aspects of the Tregitope-blood component conjugates, the blood component is albumin, such as serum albumin, human serum albumin, recombinant albumin, and recombinant human serum albumin. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys34) (the location of the amino acid in the amino acid sequence of human serine albumin), containing a free thiol with a pKa of approximately 5, which may serve as a preferred reactive functionality of albumin. Cys34 of albumin is capable of forming a stable thioester bond with maleimidopropionamido (MPA), which is a preferred reactive moiety of a modified Tregitope peptide.

In aspects, reactive functionalities on the blood component of the Tregitope-blood component conjugates or on the blood components that are capable of forming a conjugate with the instantly-disclosed modified polypeptides are groups on blood components, including mobile and fixed proteins, to which reactive groups on modified therapeutic peptides react to form covalent bonds. As disclosed in U.S. Pat. Nos. 6,849,714, 6,887,470, 7,256,253, and 7,307,148, such functionalities usually include hydroxyl groups for bonding to ester reactive groups, thiol groups for bonding to maleimides, imidates and thioester groups; amino groups for bonding to activated carboxyl, phosphoryl or any other acyl groups on reactive groups. In aspects, the reactive functionality of the blood component is an amino group, a hydroxyl group, or a thiol group. In aspects, the reactive functionality of the blood component is a component of a side group of an amino acid in a polypeptide and/or protein, wherein the reactive functionality is near the surface of the polypeptide and/or protein. In aspects, the reactive functionality of the blood component is a thiol group of a free cysteine residue of a proteinaceous blood component. In aspects, the reactive functionality is a free thiol group of the cysteine at amino acid 34 (Cys³⁴) of serine albumin. In aspects, the reactive functionality of the blood component is a thiol with a pKa of approximately 5 in a physiological environment, such as plasma. In aspects, the reactive functionality of the blood component is a thiol with a pKa of approximately 5.5 in a physiological environment, such as plasma. In aspects, the reactive functionality of the blood component is a thiol with a pKa of 3-7 in a physiological environment, such as plasma. In aspects, the reactive functionality of the blood component is a thiolate anion. In aspects, the reactive functionality is a thiolate anion of the cysteine at amino acid 34 (Cys³⁴) of serine albumin.

In aspects, the modified polypeptides of the Tregitope-blood component conjugates and the modified polypeptides used to form the Tregitope-blood component conjugates comprise a reactive moiety that is attached to the polypeptide, with the reactive moiety being capable of forming a bond (e.g., a covalent linkage) with a reactive functionality on the blood component. In aspects, the reactive group is capable of reacting with an amino group, a hydroxyl group, or a thiol group on blood component to form a covalent bond therewith. In aspects, the reactive moiety is placed at a site such that when the modified polypeptide is bonded to the blood component, the modified peptide retains a substantial proportion of the parent compound's activity. In aspects, the reactive moiety may be a succinimidyl or maleimido group. In aspects, the reactive moiety may be attached to an amino acid positioned in the less therapeutically active region of amino acids of the polypeptide to be modified. In aspects, the reactive moiety is attached to the amino terminal amino acid of the modified polypeptide. In aspects, the reactive moiety is attached to the carboxy terminal amino acid of the modified polypeptide. In aspects, the reactive moiety is attached to an amino acid positioned between the amino terminal amino acid and the carboxy terminal amino acid of the modified polypeptide. In aspects, the reactive group may be attached to the polypeptide (to be modified) either via a linking group, or optionally without using a linking group. Further, one or more additional amino acids (e.g., one or more lysines) may be added to the polypeptide to facilitate the attachment of the reactive group. Linking groups are chemical moieties that link or connect reactive groups of blood components to polypeptides comprising one or more Tregitopes. Linking groups may comprise one or more alkyl groups, alkoxy group, alkenyl group, alkynyl group or amino group substituted by alkyl groups, cycloalkyl group, polycyclic group, aryl groups, polyaryl groups, substituted aryl groups, heterocyclic groups, and substituted heterocyclic groups. Linking groups may also comprise poly ethoxy aminoacids such as AEA ((2-amino)ethoxy acetic acid) or a preferred linking group AEEA ([2-(2-amino)ethoxy)]ethoxy acetic acid). In aspects, linking groups may comprise a polyethyleneglycol linker (e.g. but not limited to, PEG2 or PEG12).

As should be understood, modified polypeptides may be administered in vivo such that conjugation with blood components occurs in vivo, or they may be first conjugated to blood components in vitro and the resulting peptidase stabilized polypeptide administered in vivo. Further, as disclosed in in U.S. Pat. Nos. 6,849,714, 6,887,470, 7,256,253, and 7,307,148, a peptidase stabilized polypeptide is a modified polypeptide that has been conjugated to a blood component via a covalent bond formed between the reactive group of the modified peptide and the functionalities of the blood component, with or without a linking group. Such reaction is preferably established by covalent bonding of a polypeptide modified with a maleimide link (e.g. prepared from GMBS, MPA or other maleimides) to a thiol group on a mobile blood protein such as serum albumin or IgG. Peptidase stabilized polypeptides are more stable in the presence of peptidases in vivo than a non-stabilized peptide. A peptidase stabilized therapeutic peptide generally has an increased half-life of at least 10-50% as compared to a non-stabilized peptide of identical sequence. Peptidase stability is determined by comparing the half-life of the unmodified therapeutic peptide in serum or blood to the half-life of a modified counterpart therapeutic peptide in serum or blood. Half-life is determined by sampling the serum or blood after administration of the modified and non-modified peptides and determining the activity of the peptide. In addition to determining the activity, the length of the therapeutic peptide may also be measured.

In aspects, the modified polypeptides of the Tregitope-blood component conjugates and the modified polypeptides used to form the Tregitope-blood component conjugates comprise one or more Tregitopes as disclosed herein. In aspects, the one or more Tregitopes of the modified polypeptides have a sequence comprising, consisting of, or consisting essentially of one or more of SEQ ID NOS: 1-73 (and fragments and variants thereof) as essentially disclosed herein. In aspects, the one or more Tregitopes of the modified polypeptide may optionally have one or more linkers, which may optionally be cleavage sensitive sites, adjacent to their N and/or C terminal end. In such a modified polypeptide, two or more of the Tregitopes may have a cleavage sensitive site between them. Alternatively, two or more of the Tregitopes may be connected directly to one another or through a linker that is not a cleavage sensitive site. In aspects, the modified polypeptide comprising the one or more Tregitopes and/or the Tregitopes contained therein can be either in neutral (uncharged) or salt forms, and may be either free of or include modifications such as glycosylation, side chain oxidation, or phosphorylation. In certain aspects, the modified polypeptide comprising the one or more Tregitopes peptides or polypeptides can be capped with an n-terminal acetyl and/or c-terminal amino group. In aspects, the one or more Tregitopes included in the modified polypeptide can be capped with an n-terminal acetyl and/or c-terminal amino group.

In aspects, the blood component that forms the Tregitope-blood component conjugate with the modified Tregitope is albumin. In aspects, the reactive functionality of the blood component is an amino group, a hydroxyl group, or a thiol group. In aspects, the reactive functionality of the blood component is a component of a side group of an amino acid in a polypeptide and/or protein, wherein the reactive functionality is near the surface of the polypeptide and/or protein. In aspects, the reactive functionality of the blood component is a thiol group of a free cysteine residue of a proteinaceous blood component. In aspects, the reactive functionality is a free thiol group of the cysteine at amino acid 34 (Cys³⁴) of serine albumin. In aspects, the reactive functionality of the blood component is a thiol with a pKa of approximately 5 in a physiological environment, such as plasma. In aspects, the reactive functionality of the blood component is a thiol with a pKa of approximately 5.5 in a physiological environment, such as plasma. In aspects, the reactive functionality of the blood component is a thiol with a pKa of 3-7 in a physiological environment, such as plasma. In aspects, the reactive functionality of the blood component is a thiolate anion. In aspects, the reactive functionality is a thiolate anion of the cysteine at amino acid 34 (Cys³⁴) of serine albumin.

In aspects, the reactive moiety of the modified Tregitope peptide is a soft electrophile. In aspects, the reactive moiety of the modified Tregitope peptide is an electrophile selective for thiols. In a preferred embodiment, the reactive moiety attached to the Tregitope to create the modified Tregitope peptide is maleimide. In aspects, the reactive moiety is maleimide propionic acid. In a preferred embodiment, the reactive moiety attached of the modified Tregitope peptide is maleimide, the blood component is albumin, and the reactive functionality on the albumin is a free thiol or thiolate anion of Cys³⁴ of albumin. When the reactive moiety of the modified Tregitope peptide a maleimide, the blood component is albumin, and the reactive functionality of the albumin is a free thiol or thiolate anion of Cys³⁴ of albumin, a stable thioester linkage between the maleimide group and the sulfhydryl is formed which cannot be cleaved under physiological conditions. In aspects, the modified Tregitope peptide contains a linker, wherein the reactive moiety is attached to the Tregitope peptide through the linker. In aspects, the modified Tregitope peptide binds to the blood component in a 1:1 molar ratio.

The manner of producing the modified Tregitope peptides of the present disclosure will vary widely, depending upon the nature of the various elements comprising the molecule. The synthetic procedures may be selected so as to be simple, provide for high yields, and allow for a highly purified stable product. Normally, the reactive moiety will be created as the last stage, for example, with a carboxyl group, esterification to form an active ester will be the last step of the synthesis.

In aspects, the present disclosure is also directed to a method of synthesizing the modified Tregitope peptide, as disclosed in U.S. Pat. Nos. 6,849,714, 6,887,470, 7,256,253, and 7,307,148. In aspects, the method comprises the following steps. In the first step, the one or more Tregitope sequence of the polypeptide can be as essentially disclosed herein. In the second step, if the polypeptide does not contain a cysteine, then the polypeptide may be synthesized from the carboxy terminal amino acid and the reactive moiety is added to the carboxy terminal amino acid. Alternatively, a terminal lysine (or one or more lysines) may added to the carboxy terminal amino acid and the reactive moiety is added to the terminal lysine. In the third step, if the polypeptide contains only one cysteine, then the cysteine is reacted with a protective group prior to addition of the reactive moiety to an amino acid in a less therapeutically active region of the polypeptide. In the fourth step, if the polypeptide contains two cysteines as a disulfide bridge, then the two cysteines are oxidized and the reactive moiety is added to the amino terminal amino acid, or to the carboxy terminal amino acid, or to an amino acid positioned between the carboxy terminal amino acid and the amino terminal amino acid of the polypeptide. In the fifth step, if the polypeptide contains more than two cysteines as disulfide bridges, the cysteines are sequentially oxidized in the disulfide bridges and the peptide is purified prior to the addition of the reactive moieties to the carboxy terminal amino acid.

In aspects, the present disclosure is also directed to a method of synthesizing the Tregitope-blood component conjugate. In a first step, reactive maleimidopropionamido (MPA) is added via an N-terminal lysine on the polypeptide comprising one or more Tregitopes to create a modified polypeptide. In aspects, one or more lysines are present on the N-terminus of the polypeptide, optionally present at the N-terminus of a Tregitope sequence selected from the group of SEQ. ID NOS: 1-73 as disclosed herein. Optionally, polyethyleneglycol linker, such as PEG2 or PEG12, is present between the one or more lysines and a Tregitope sequence, or at the N-terminus of a Tregitope sequence. In aspects, a lysosomal cleavage site, such as a Cathepsin B site, optionally consisting (sequentially from N-terminus to C-terminus) of valine and citrulline, is present between the PEG2 or PEG12 moiety and the Tregitope sequence. The lysosomal cleavage site (such as Cathepsin B site) may be incorporated to provide a lysosomal protease site, allowing the Tregitope to be released into the lysosomal compartment. In aspects, lysosomal cleavage site (such as Cathepsin B site) is present to provide a lysosomal protease site, allowing the Tregitope to be released into cells, preferably into the early endosome. In a preferred embodiment, the lysosomal cleavage site (such as Cathepsin B site) is present to provide a lysosomal protease site, allowing the Tregitope to be released into cells, such as into a membrane-enclosed vesicle (such as the early endosome, late endosome, or lysosome), such that the Tregitope may be processed for antigen presentation. In aspects, the Tregitope is presented as antigen by immune cells, such as macrophages or antigen-presenting cells, preferably presented as an MHC class II antigen. In aspects, a lysosomal cleavage site, such as a Cathepsin B site, optionally consisting (sequentially from N-terminus to C-terminus) of valine and citrulline, is present between the PEG2 moiety and the Tregitope sequence, and/or between one or more Tregitopes. In aspects, one or more Tregitopes may be present on the construct, optionally more proximate to the C-terminus than the linker. In aspects, one or more lysosomal cleavage sites are present between multiple Tregitopes (for example, such that a single lysosomal cleavage site separates two Tregitopes, or such that one lysosomal cleavage site is present between a first and second Tregitope, and another lysosomal cleavage site is present between a second and third Tregitope, and so on). In aspects, a norleucine (Nle) residue is present at the C-terminus as a means to quantitate the amount of Tregitope peptide incorporated into the final Tregitope-blood component conjugate, for example for evaluation by mass spectrometry. In aspects, the C-terminus of the polypeptide is capped with a c-terminal amino group. In a second step, a maleimide-based chemistry is used to covalently link the modified polypeptide to a blood component, preferably serum albumin, in a 1:1 molar ratio. The second step may be performed in vivo or ex vivo, as described further below and in the examples of the present disclosure.

In aspects, the formation of the Tregitope-blood component conjugate protects the Tregitope, when present in vivo, from rapid degradation by peptidases, rapid clearance from circulation, and/or rapid kidney excretion. In aspects, the formation of the Tregitope-blood component conjugate significantly extends the half-life of the Tregitope in vivo. In aspects, the formation of the Tregitope-blood component conjugate allows wide distribution of the Tregitope-blood component conjugate throughout the body of a subject. In aspects, the Tregitope-blood component conjugate does not cross the blood-brain barrier when present in the plasma of a subject. In aspects, the Tregitope-blood component conjugate aid in delivery of Tregitopes to appropriate immune cells, such as macrophages and/or antigen-presenting cells (APCs). In aspects, upon delivery of Tregitopes to appropriate immune cells, such as macrophages and/or APCs, the Tregitopes are encompassed in a membrane-bound vesicle, preferably a vesicle in the endocytic pathway such as an early endosome, late endosome, or lysosome. In aspects, the Tregitopes, once processed by the appropriate immune cells, such as macrophages and/or APCs, are presented as MHC class II antigens.

In aspects, the Tregitope in the Tregitope-blood component conjugate has a plasma half-life in vivo of up to 12 hours. In aspects, the Tregitope in the Tregitope-blood component conjugate has a plasma half-life in vivo of up to 1 day. In aspects, the Tregitope in the Tregitope-blood component conjugate has a plasma half-life in vivo of up to 40-48 hours. In aspects, the Tregitope in the Tregitope-blood component conjugate has a plasma half-life in vivo of up to 60 hours. In aspects, the Tregitope in the Tregitope-blood component conjugate has a plasma half-life in vivo of up to 15 days.

In aspects, the modified polypeptide comprising one or more Tregitopes is administered to a subject, wherein upon administration, the modified polypeptide reacts in vivo with a reactive functionality of a circulating blood component. In aspects, the peptide is administered to a human subject, and the blood component is human albumin, preferably the circulating albumin of the human subject.

In aspects, the modified polypeptides used to form the Tregitope-blood component conjugates is capable of forming a bond ex vivo with a reactive functionality on a blood component, wherein upon formation of a bond between the reactive moiety of the modified polypeptide and the reactive functionality on the blood component, a Tregitope-blood component conjugate is formed, as disclosed in U.S. Pat. Nos. 6,849,714, 6,887,470, 7,256,253, and 7,307,148. In aspects, the modified polypeptide as disclosed herein is configured to covalently attach to a reactive functionality of a blood component outside of the body. In aspects, the blood component is albumin. In aspects, the blood component is selected from the group of recombinant albumin, human recombinant albumin, and albumin from a genomic source.

In aspects, the present disclosure is also directed to an ex vivo method of synthesizing the modified Tregitope peptide and the Tregitope-blood component conjugate, as disclosed in U.S. Pat. Nos. 6,849,714, 6,887,470, 7,256,253, and 7,307,148. In aspects, the modified polypeptide as disclosed herein is added to blood, serum or saline solution containing human serum albumin to permit covalent bond formation between the modified therapeutic peptide and the blood component. In aspects, the polypeptide comprising one or more Tregitopes as disclosed herein is modified with maleimide and it is reacted with serum albumin in saline solution. In aspects, once the modified polypeptide has reacted with the blood component, to form a Tregitope-blood component conjugate, the conjugate may be administered to the subject. In aspects, after the modified polypeptide has reacted with the blood component to form the conjugate, but before the conjugate is administered to the subject, the conjugate may be separated from non-conjugated blood components in the reaction mixture. In aspects, conjugate may be separated from non-conjugated blood components in the reaction mixture by separating substances on the basis of their varying strengths of hydrophobic interactions with hydrophobic ligands immobilized to an uncharged matrix. In aspects, the uncharged matrix may be a hydrophobic solid support, wherein the support comprises a column containing a hydrophobic resin such as, but not limited to, octyl sepharose, phenyl sepharose and butyl sepharose. In aspects, this technique may be performed with moderately high concentrations of salts (≈1 M) in the start buffer (salt promoted adsorption). Elution is achieved by a linear or stepwise decrease in salt concentration. The type of ligand, the degree of substitution, the pH and the type and concentration of salt used during the adsorption stage have a profound effect on the overall performance (e.g. selectivity and capacity) of a HIC matrix (Hydrophobic Interaction Chromatography matrix).

The solvent is one of the most important parameters which influence capacity and selectivity in HIC (Hydrophobic Interaction Chromatography). In general, the adsorption process is more selective than the desorption process. It is therefore important to optimize the start buffer with respect to pH, type of solvent, type of salt and concentration of salt. The addition of various “salting-out” salts to the sample promotes ligand-protein interactions in HIC. As the concentration of salt is increased, the amount of bound protein increases up to the precipitation point for the protein. Each type of salt differs in its ability to promote hydrophobic interactions.

Increasing the salting-out effect strengthens the hydrophobic interactions, whereas increasing the chaotropic effect weakens them. Examples of salts with high salting-out effects, in order from greater salting-out effect to smaller salting-out effect, include: PO₄³⁻, SO₄²⁻, CH₃COO⁻, Cl⁻, Br⁻, NO₃⁻, ClO₄⁻, I⁻, and SCN⁻. Examples of salts with high chaotropic effects, in order from greater chaotropic effect to smaller chaotropic effect, include: NH₄⁺, Rb⁺, K⁺, Na⁺, Cs⁺, Li⁺, Mg²⁺, and Ba²⁺. The most commonly used salts for HIC are ammonium sulfate ((NH₄)₂SO₄), sodium sulfate ((Na)₂SO₄)), magnesium sulfate (MgSO₄), sodium chloride (NaCl), potassium chloride (KCl), and ammonium acetate (CH₃COONH₄).

Protein binding to HIC adsorbents is promoted by moderate to high concentrations of “salting-out” salts, most of which also have a stabilizing influence on protein structure due to their preferential exclusion from native globular proteins, i.e. the interaction between the salt and the protein surface is thermodynamically unfavorable. The salt concentration should be high enough (e.g. 500-1000 mM) to promote ligand-protein interactions yet below that which causes precipitation of the protein in the sample. In the case of albumin, the salt concentration should be kept below 3M (moles per liter). The principle mechanism of salting-out consists of the salt-induced increase of the surface tension of water (Melander and Horvath, 1977). Thus, a compact structure becomes energetically more favorable because it corresponds to smaller protein-solution interfacial area. Under these conditions, for example buffer composed of SO₄²⁻, PO₄²⁻ or CH₃COO⁻ with any counter ion, these salts exhibit their salting-out effect upon essentially all conjugated albumin described herein in a manner different to non-conjugated albumin (i.e. mercaptalbumin and albumin capped with cysteine), thus enabling a consistent chromatographic separation between conjugated albumin versus non-conjugated albumin. Thus, lower concentrations of salt are required to promote interactions between ligand and conjugated albumin than between ligand and non-conjugated albumin. This chromatographic separation is essentially independent of (a) the sequence of albumin (e.g. human, mouse, rat, etc.) (b) the source of albumin (i.e. plasma derived or recombinant) (c) the molecular weight of the conjugated modified Tregitope, (d) the position of the reactive moiety within the structure of the molecule, (e) the peptide sequence or chemical structure of the molecule, and (f) the three-dimensional structure of the conjugated molecule, e.g. linear versus loop structure.

In aspects, the salt of the aqueous buffer has a sufficient salting-out effect. In aspects, for providing a sufficient salting out effect, the salt may be phosphate, sulfate and acetate. In aspects, the selection of the cation of the buffer is can be selected, without limitation, from the group consisting of NH₄⁺, Rb⁺, K⁺, Na⁺, Cs⁺, Li⁺, Mg²⁺ and Ba²⁺. In aspects, the aqueous buffer may be selected from the group of ammonium phosphate, ammonium sulfate and magnesium phosphate. In aspects, the buffer pH is between 3.0 and 9.0; more preferably between 6.0 and 8.0, and even more preferably, the pH is 7.0. In aspects, the buffer and the hydrophobic solid support are at room temperature (about 25° C.) or at 4° C. or in between.

In aspects, the present disclosure also provides chimeric or fusion polypeptide compositions. In aspects, the present disclosure provides isolated, synthetic, or recombinant chimeric or fusion polypeptide compositions wherein one or more of the instantly-disclosed Tregitopes is a part thereof. In aspects, a chimeric or fusion polypeptide composition comprises one or more polypeptides (Treg activating regulatory T-cell epitope, Tregitope, Tregitope peptide, or T-cell epitope polypeptide) of the present disclosure joined to, linked to (e.g., fused in-frame, chemically-linked, or otherwise bound), and/or inserted into a heterologous polypeptide (e.g. but not limited to, an antibody (which can be IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody)). As previously described, with respect to the one or more Tregitopes of the instant disclosure, the term “heterologous polypeptide” is intended to mean that the one or more Tregitopes of the instant disclosure are heterologous to, or not included naturally, in the heterologous polypeptide. In aspects, one or more of the instantly-disclosed polypeptides (T_{reg} activating regulatory T-cell epitopes, Tregitopes, or T-cell epitope polypeptides) may be inserted into the heterologous polypeptide (e.g., through recombinant techniques, mutagenesis techniques, or other known means in the art), may be added to the C-terminus, and/or added to the N-terminus of the heterologous polypeptide. For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, the one or more Tregitopes may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). In aspects, chimeric or fusion polypeptides comprise one or more of the instantly-disclosed polypeptides (T_{reg} activating regulatory T-cell epitopes, Tregitopes, or T-cell epitope polypeptides) operatively linked to a heterologous polypeptide. “Operatively linked” indicates that the one or more of the instantly-disclosed polypeptides (T_{reg} activating regulatory T-cell epitopes, Tregitopes, or T-cell epitope polypeptides) and the heterologous polypeptide are fused in-frame or chemically-linked or otherwise bound.

In aspects of the above isolated, synthetic, or recombinant chimeric or fusion polypeptide compositions, the one or more polypeptides (Treg activating regulatory T-cell epitope, Tregitope, Tregitope peptide, or T-cell epitope polypeptide) of the present disclosure have a sequence comprising, consisting of, or consisting essentially of one or more of SEQ ID NOS: 1-73. In aspects of the instantly-disclosed chimeric or fusion polypeptide compositions, the one or more polypeptides comprise, consist, or consist essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73. In aspects of the chimeric or fusion polypeptide compositions, the one or more Tregitopes as disclosed herein may be joined to, linked to (e.g., fused in-frame, chemically-linked, or otherwise bound), and/or inserted into a heterologous polypeptide as a whole, although it may be made up from a joined to, linked to (e.g., fused in-frame, chemically-linked, or otherwise bound), and/or inserted amino acid sequence, together with flanking amino acids of the heterologous polypeptide. In aspects, a chimeric or fusion polypeptide composition comprises a polypeptide, said polypeptide having a sequence comprising one or more of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73 of the present disclosure, wherein said one or more of SEQ ID NOS: 1-73 is not naturally included in the polypeptide and/or said of one or more of SEQ ID NOS: 1-73 is not located at its natural position in the polypeptide. In aspects, the one or more Tregitopes of the present disclosure can be joined, linked to (e.g., fused in-frame, chemically-linked, or otherwise bound), and/or inserted into the polypeptide. In aspects, chimeric or fusion polypeptide compositions comprise one or more of the instantly-disclosed Tregitopes operatively linked to a heterologous polypeptide having an amino acid sequence not substantially homologous to the Tregitope. In aspects, the chimeric or fusion polypeptide does not affect function of the Tregitope per se. For example, the fusion polypeptide can be a GST-fusion polypeptide in which the Tregitope sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Ig fusions. Such fusion polypeptides, particularly poly-His fusions or affinity tag fusions, can facilitate the purification of recombinant polypeptide. In certain host cells (e.g., mammalian host cells), expression and/or secretion of a polypeptide can be increased by using a heterologous signal sequence. Therefore, in aspects, the fusion polypeptide contains a heterologous signal sequence at its N-terminus. In aspects of the above recombinant chimeric or fusion polypeptide compositions, the heterologous polypeptide or polypeptide comprises a biologically active molecule. In aspects, the biologically active molecule is selected from the group consisting of an immunogenic molecule, a T cell epitope, a viral protein, and a bacterial protein. In aspects, the biologically active molecule is a rhGAA. In aspects, the one or more of SEQ ID NOS: 1-73 of the present disclosure can be joined or linked to (e.g., fused in-frame, chemically-linked, or otherwise bound) to a small molecule, drug, or drug fragment. For example, one or more of SEQ ID NOS. 1-73 (and/or fragments or variants thereof, and optionally 1 to 12 additional amino acids distributed in any ratio on the N terminus and/or C-terminus of the polypeptide of SEQ ID NOS. 1-73) of the present disclosure can be joined or linked to (e.g., fused in-frame, chemically-linked, or otherwise bound) a drug or drug fragment that is binds with high affinity to defined HLAs. In aspects of the above chimeric or fusion polypeptide compositions or fusion products, the one or more polypeptides (Treg activating regulatory T-cell epitope, Tregitope, Tregitope peptide, or T-cell epitope polypeptide) of the present disclosure included therein have a sequence comprising, consisting of, or consisting essentially of one or more of SEQ ID NOS: 1-73. In aspects of the above chimeric or fusion polypeptide compositions or fusion products, the one or more polypeptides (Treg activating regulatory T-cell epitope, Tregitope, Tregitope peptide, or T-cell epitope polypeptide) of the present disclosure included therein have a sequence comprising, consisting of, or consisting essentially of one or more of SEQ ID NOS: 1-6. In aspects of the above chimeric or fusion polypeptide compositions or fusion products, the one or more polypeptides (Treg activating regulatory T-cell epitope, Tregitope, Tregitope peptide, or T-cell epitope polypeptide) of the present disclosure included therein have a sequence comprising, consisting of, or consisting essentially of SEQ ID NOS: 1. In aspects of the above-described chimeric or fusion polypeptide compositions or fusion products, the chimeric or fusion polypeptide compositions or fusion products can be recombinant, isolated, and/or synthetic.

A chimeric or fusion polypeptide composition can be produced by standard recombinant DNA or RNA techniques as are known in the art. For example, DNA or RNA fragments coding for the different polypeptide sequences may be ligated together in-frame in accordance with conventional techniques. In another embodiment, the fusion gene can be synthesized by conventional techniques including automated DNA synthesizers. Alternatively, polymerase chain reaction (PCR) amplification of nucleic acid fragments can be carried out using anchor primers which give rise to complementary overhangs between two consecutive nucleic acid fragments which can subsequently be annealed and re-amplified to generate a chimeric nucleic acid sequence. (Short Protocols in Molecular Biology: A Compendium of Methods from Current Protocols in Molecular Biology, (2^{ND}, 1992), F M Asubel et al. (eds), Green Publication Associates, New York, NY (Publ), ISBN: 9780471566355, which are herein incorporated by reference in their entirety). Further, one or more polypeptides (Treg activating regulatory T-cell epitope, Tregitope, Tregitope peptide, or T-cell epitope polypeptide) of the present disclosure (e.g., one or more Tregitopes of the present disclosure having a sequence comprising, consisting of, or consisting essentially of one or more of SEQ ID NOS: 1-73) can be inserted into a heterologous polypeptide or inserted into a non-naturally occurring position of a polypeptide through recombinant techniques, synthetic polymerization techniques, mutagenesis techniques, or other standard techniques known in the art. For example, protein engineering by mutagenesis can be performed using site-directed mutagenesis techniques, or other mutagenesis techniques known in the art (see e.g., James A. Brannigan and Anthony J. Wilkinson, 2002, Protein engineering 20 years on. Nature Reviews Molecular Cell Biology 3, 964-970; Turanli-Yildiz B. et al., 2012, Protein Engineering Methods and Applications, intechopen.com, which are herein incorporated by reference in their entirety). In aspects, the one or more Tregitopes may be inserted into or replace amino acids in a Fc domain as disclosed in U.S. Pat. Nos. 7,442,778, 7,645,861, 7,655,764, 7,655,765, and/or 7,750,128 (each of which are herein incorporated by reference in their entirety). In aspects, the one or more Tregitopes may be covalently bound to one or more internal conjugation site(s) in a Fc domain as disclosed in U.S. Pat. Nos. 8,008,453, 9,114,175, and/or 10,188,740 (each of which are herein incorporated by reference in their entirety).

In some aspects, the chimeric or fusion proteins or polypeptides feature one or more polypeptides (Treg activating regulatory T-cell epitope, Tregitope, Tregitope peptide, or T-cell epitope polypeptide) of the present disclosure included therein have a sequence comprising, consisting of, or consisting essentially of one or more of SEQ ID NOS: 1-73 fused to or inserted in a human GAA sequence (SEQ ID NO: 101) or a variant thereof. Natural variants of SEQ ID NO: 101 can also be utilized in the fusion or chimeric proteins, including one or more of the following variations: S46P, C103G, C103R, C108G, C127F, R190H, Y191C, L208P, P217L, G219RR224P, R224Q, R224W, T234K, T234R, A237V, S251L, S254L, E262K, P266S, P285R, P285S, L291F, L291P, Y292C, G293R, L299R, H308L, H308P, G309R, L312R, N316I, M318K, M318T, P324L, W330G, G335E, G335R, P347R, L355P, P361L, C374R, R375L, G377R, P397L, Q401R, W402R, D404N, L405P, M408V, D419V, 431-433 missing, R437C, R437H, A445P, Y455F, PP457H, P457L, 459 missing, G478R, W481R, P482R, G483V, A486P, D489N, M519T, M519V, E521K, E521Q, P522A, P522S, S523Y, F525Y, S529V, P545L, G549R, L552P, 1557F, C558S, S566P, H568L, N570K, H572Q, Y575C, Y575S, G576R, E579K, R585M, R594H, R594P, S599Y, R6000, R600H, S601L, T602A, 607-612 missing, G607D, A610P, H612Y, H612Y, T614K, G615R, S619R, S627P, N635K, G638V, G638W, L641V, G643R, D645E, D645H, C647W, G648D, G648S, R660H, R672Q, R672T, R672W, 675 missing, R702C, R702L, L705P, R725W, T737N, Q743K, W746G, W746S, Y766C, P768R, R819P, A880D, L901Q, 903 missing, P913R, V916F, N925NGVPVSN, L935P, V949D and combinations thereof. By way of example, any 1 or more of SEQ ID NOS: 1-6 or 9-54 can be fused to an amino terminus or a carboxy terminus, with or without an addition 1-12 amino acids therebetween as a linker or incidental cloning addition (such as for enzymatic restriction or to maintain reading frame etc.). Any one of SEQ ID NOS: 1-73 may also be fused or inserted internally (e.g., but not limited to, site directed mutagenesis or other recombinant techniques) within SEQ ID NO: 101, such as in instances where the subjects own GAA is missing such a Tregitope (e.g., if a particular patient has a mutated or missing corresponding section in their own expressed GAA) By way of example, SEQ ID NO: 101 or a variant thereof may be fused at either or both termini and/or internally with one or more of:

(SEQ ID NO: 1);
SHRLLAVCALVSLATAA;
(SEQ ID NO: 2)
SHRLLAVCALVSLATAALLGHI;
(SEQ ID NO: 3)
AAALVSLATAALLGHI;
(SEQ ID NO: 4)
KDILTLRLDVMMETE;
(SEQ ID NO: 5)
AHGVFLLNSNAMDVVLQ;
(SEQ ID NO: 6)
GAGLQLQKVTVLGVATAPQQVL;
(SEQ ID NO: 12)
LLAVCALVS;
(SEQ ID NO: 15)
VCALVSLAT;
(SEQ ID NO: 18)
LVSLATAAL;
(SEQ ID NO: 19)
VSLATAALL;
(SEQ ID NO: 33)
VFLLNSNAM;
(SEQ ID NO: 34)
FLLNSNAMD;
(SEQ ID NO: 35)
LLSNNAMDV;
(SEQ ID NO: 42)
LQLQKVTVL;
(SEQ ID NO: 43)
QLQKVTVLG.

Moreover, many expression vectors are commercially available that already encode a fusion moiety (e.g., a GST protein). A nucleic acid molecule encoding a Tregitope of the invention can be cloned into such an expression vector such that the fusion moiety is linked in-frame to the at least one Tregitope.

In aspects, the polypeptides, concatemeric polypeptides, and chimeric or fusion polypeptides can be purified to homogeneity or partially purified. It is understood, however, that preparations in which the Tregitope compounds and compositions are not purified to homogeneity are useful. The critical feature is that the preparation allows for the desired function of the Tregitope, even in the presence of considerable amounts of other components. Thus, the present disclosure encompasses various degrees of purity. In one embodiment, the language “substantially free of cellular material” includes preparations of the Tregitope having less than about 30% (by dry weight) other proteins (e.g., contaminating protein), less than about 20% other proteins, less than about 10% other proteins, less than about 5% other proteins, less than about 4% other proteins, less than about 3% other proteins, less than about 2% other proteins, less than about 1% other proteins, or any value or range therebetween.

In aspects, when a polypeptide, concatemeric polypeptide, and chimeric or fusion polypeptide of the present disclosure is recombinantly produced, the composition can also be substantially free of culture medium, for example, culture medium represents less than about 20%, less than about 10%, or less than about 5% of the volume of the polypeptides, concatemeric polypeptides, and chimeric or fusion polypeptides preparation. The language “substantially free of chemical precursors or other chemicals” includes preparations of the polypeptides, concatemeric polypeptides, and chimeric or fusion polypeptides in which it is separated from chemical precursors or other chemicals that are involved in the T-cell epitope's synthesis. The language “substantially free of chemical precursors or other chemicals” can include, for example, preparations of the polypeptides, concatemeric polypeptides, and chimeric or fusion polypeptides having less than about 30% (by dry weight) chemical precursors or other chemicals, less than about 20% chemical precursors or other chemicals, less than about 10% chemical precursors or other chemicals, less than about 5% chemical precursors or other chemicals, less than about 4% chemical precursors or other chemicals, less than about 3% chemical precursors or other chemicals, less than about 2% chemical precursors or other chemicals, or less than about 1% chemical precursors or other chemicals.

In aspects, the present disclosure also includes pharmaceutically acceptable salts of the Regulatory T-cell epitope compounds and compositions (including one or more of e.g., peptides or polypeptides as disclosed herein; concatemeric peptides as disclosed herein; chimeric or fusion polypeptide compositions as disclosed herein (which in aspects may be isolated, synthetic, and/or recombinant). “Pharmaceutically acceptable salt” means a salt that is pharmaceutically acceptable and that possesses the desired pharmacological activity of the parent peptide or polypeptide (e.g., peptides, polypeptides, concatemeric peptides, and/or chimeric or fusion polypeptides as disclosed herein). As used herein, “pharmaceutically acceptable salt” refers to derivative of the instantly-disclosed polypeptides, concatemeric polypeptides, and/or chimeric or fusion polypeptides, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycoloylarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

### Nucleic Acids

In aspects, the present disclosure also provides for nucleic acids (e.g., DNA (such as, but not limited to cDNA) and RNA (such as, but limited to mRNA)), vectors, viruses, or hybrids thereof, all of which may be isolated, synthetic, or recombinant) that encode in whole or in part one or more polypeptides, concatemeric peptides, and/or chimeric or fusion polypeptides of the present disclosure as described herein. In aspects, the nucleic acid further comprises, or is contained within, an expression cassette, a plasmid, and expression vector, or recombinant virus, wherein optionally the nucleic acid, or the expression cassette, plasmid, expression vector, or recombinant virus is contained within a cell, optionally a human cell or a non-human cell, and optionally the cell is transformed with the nucleic acid, or the expression cassette, plasmid, expression vector, or recombinant virus. In aspects, cells are transduced, transfected, or otherwise engineered to contain within one or more of e.g., polypeptides of the present disclosure; isolated, synthetic, or recombinant nucleic acids, expression cassettes, plasmids, expression vectors, or recombinant viruses as disclosed herein; and/or isolated, synthetic, or recombinant chimeric or fusion polypeptide compositions as disclosed herein. In aspects, the cell can be a mammalian cell, bacterial cell, insect cell, or yeast cell. In aspects, the nucleic acid molecules of the present disclosure can be inserted into vectors and used, for example, as expression vectors or gene therapy vectors. Gene therapy vectors can be delivered to a subject by, e.g., intravenous injection, local administration (U.S. Pat. No. 5,328,470) or by stereotactic injection (Chen S H et al., (1994), Proc Natl Acad Sci USA, 91(8):3054-7, which are herein incorporated by reference in their entirety). Similarly, the nucleic acid molecules of the present disclosure can be inserted into plasmids. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. Such pharmaceutical compositions can be included in a container, pack, or dispenser together with instructions for administration. In aspects of the above nucleic acids (e.g., DNAs, RNAs, vectors, viruses, or hybrids thereof) that encode in whole or in part at least one or more peptides, polypeptides, concatemeric peptides, and/or chimeric or fusion polypeptides as described herein, the nucleic acids encode one or more peptides or polypeptides of the instant disclosure as described above (e.g., but not limited to, a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73; as well as the concatemeric peptides as disclosed herein. In aspects, the present disclosure is directed to a vector comprising a nucleic acid of the present disclosure encoding one or more polypeptides of the present disclosure or chimeric or fusion polypeptide composition of the present disclosure. In aspects, the present disclosure is directed to a cell comprising a vector of the present disclosure. In aspects, the cell can be a mammalian cell, bacterial cell, insect cell, or yeast cell.

The nucleic acid of the instant disclosure may be DNAs (including but not limited to cDNA) or RNAs (including but not limited to mRNA), single- or double-stranded. The nucleic acid is typically DNA or RNA (including mRNA). The nucleic acid may be produced by techniques well known in the art, such as synthesis, or cloning, or amplification of the sequence encoding the immunogenic polypeptide; synthesis, or cloning, or amplification of the sequence encoding the cell membrane addressing sequence; ligation of the sequences and their cloning/amplification in appropriate vectors and cells. The nucleic acids provided herein (whether RNAs, DNAs, vectors, viruses or hybrids thereof) that encode in whole or in part one or more peptides, polypeptides, concatemeric peptides, and/or chimeric or fusion polypeptides as described herein can be isolated from a variety of sources, genetically engineered, amplified, synthetically produced, and/or expressed/generated recombinantly. Recombinant polypeptides generated from these nucleic acids can be individually isolated or cloned and tested for a desired activity. Any recombinant expression system can be used, including e.g. in vitro, bacterial, fungal, mammalian, yeast, insect or plant cell expression systems. In aspects nucleic acids provided herein are synthesized in vitro by well-known chemical synthesis techniques (as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066, all of which are herein incorporated by reference in their entirety). Further, techniques for the manipulation of nucleic acids provided herein, such as, e.g., subcloning, labeling probes (e.g., random-primer labeling using Klenow polymerase, nick translation, amplification), sequencing, hybridization and the like are well described in the scientific and patent literature (see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993), all of which are herein incorporated by reference in their entirety).

A further object of the present disclosure relates to a nucleic acid molecule encoding one or more peptides, polypeptides, concatemeric peptides, and/or chimeric or fusion polypeptides as described herein. The nucleic acid may be used to produce the one or more peptides, polypeptides, concatemeric peptides, and/or chimeric or fusion polypeptides as described herein in vitro or in vivo, or to produce cells expressing the polypeptide on their surface, or to produce vaccines wherein the active agent is the nucleic acid or a vector containing the nucleic acid. The nucleic acid may be, e.g., DNA, cDNA, PNA, CNA, RNA, either single- and/or double-stranded, or native or stabilized forms of polynucleotides as are known in the art.

As previously mentioned, the nucleic acid molecules according to the present disclosure may be provided in the form of a nucleic acid molecule per se such as naked nucleic acid molecules; a plasmid, a vector; virus or host cell, etc., either from prokaryotic or eukaryotic origin. Vectors include expression vectors that contain a nucleic acid molecule of the invention. An expression vector capable of expressing a polypeptide can be prepared. Expression vectors for different cell types are well known in the art and can be selected without undue experimentation. Generally, the (e.g., cDNA, or RNA, including mRNA) is inserted into an expression vector, such as a plasmid, in proper orientation and correct reading frame for expression. If necessary, the DNA (e.g., cDNA, or RNA, including mRNA) may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. The vectors of the present invention may, for example, comprise a transcriptional promoter, and/or a transcriptional terminator, wherein the promoter is operably linked with the nucleic acid molecule, and wherein the nucleic acid molecule is operably linked with the transcription terminator. One or more peptides or polypeptides of the present disclosure may be encoded by a single expression vector. Such nucleic acid molecules may act as vehicles for delivering peptides/polypeptides to the subject in need thereof, in vivo, in the form of, e.g., DNA/RNA vaccines.

In aspects, the vector may be a viral vector comprising a nucleic acid as defined above. The viral vector may be derived from different types of viruses, such as, Swinepox, Fowlpox, Pseudorabies, Aujezky's virus, salmonella, vaccinia virus, BHV (Bovine Herpes Virus), HVT (Herpes Virus of Turkey), adenovirus, TGEV (Transmissible Gastroenteritidis Coronavirus), Erythrovirus, and SIV (Simian Immunodeficiency Virus). Other expression systems and vectors may be used as well, such as plasmids that replicate and/or integrate in yeast cells.

The instant disclosure also relates to a method for preparing a peptide, polypeptide, concatemeric peptide, and/or chimeric or fusion polypeptide of the instant disclosure, the method comprising culturing a host cell containing a nucleic acid or vector as defined above under conditions suitable for expression of the nucleic acid and recovering the polypeptide. As indicated above, the proteins and peptides may be purified according to techniques known per se in the art.

### Pharmaceutical Compositions and Formulations

In aspects, the Tregitope compounds and compositions of the present disclosure (including one or more of e.g., polypeptides as disclosed herein; concatemeric peptides as disclosed herein; chimeric of fusion polypeptide compositions as disclosed herein; nucleic acids as disclosed herein, including nucleic acids encoding such peptides, polypeptides, concatemeric peptides, or chimeric of fusion polypeptide compositions as disclosed herein; expression cassettes, plasmids, expression vectors, and recombinant viruses, or cells as disclosed herein; hereafter referred to as “T-cell epitope compounds and compositions of the present disclosure”) may be comprised in a pharmaceutical composition or formulation. In aspects, the instantly-disclosed pharmaceutical compositions or formulations generally comprise a Tregitope compound or composition of the present disclosure and a pharmaceutically acceptable carrier, excipient, and/or adjuvant. In aspects, the instantly-disclosed pharmaceutical compositions or formulations may further comprise diluents, adjuvants, freeze drying stabilizers, wetting or emulsifying agents, pH buffering agents, gelling or viscosity enhancing additives, and preservatives, depending on the route of administration. In aspects, said pharmaceutical compositions are suitable for administration. Pharmaceutically acceptable carriers and/or excipients are determined in part by the particular composition being administered, as well as by the particular method used to administer the composition. Accordingly, there is a wide variety of suitable formulations of pharmaceutical compositions for administering the instantly disclosed Tregitope compound or compositions (see, e.g., Remington's Pharmaceutical Sciences, (18TH Ed, 1990), Mack Publishing Co., Easton, PA Publ)). In aspects, the pharmaceutical compositions are generally formulated as sterile, substantially isotonic, and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art would be able to determine the appropriate timing, sequence and dosages of administration for particular Tregitope compounds and compositions of the present disclosure. The dosage of the Tregitope compounds and compositions of the present disclosure will depend on the species, breed, age, size, treatment history, and health status of the animal (e.g., human) to be treated, as well as the route of administration, e.g., subcutaneous, intradermal, oral intramuscular or intravenous administration. The Tregitope compounds and compositions of the instant disclosure can be administered as single doses or in repeated doses. The Tregitope compounds and compositions of the instant disclosure can be administered alone, or can be administered simultaneously or sequentially administered with one or more further compositions, such as other porcine immunogenic or vaccine compositions. Where the compositions are administered at different times, the administrations may be separate from one another or overlapping in time.

Examples of pharmaceutically acceptable carriers, excipients or diluents include, but are not limited to demineralized or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

In aspects, preferred examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art. Except insofar as any conventional media or compound is incompatible with the Tregitope compounds and compositions of the present disclosure and as previously described above, use thereof in the compositions is contemplated. Supplementary active compounds can also be incorporated into the compositions.

Examples of adjuvants include, but are not limited to, oil in water emulsions, aluminum hydroxide (alum), immunostimulating complexes, non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin(s) isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde. Further adjuvants may include, but are not limited to, poly-ICLC, 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRTX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In aspects of the pharmaceutical compositions or vaccines as disclosed herein, the adjuvant comprises poly-ICLC. The TLR9 agonist CpG and the synthetic double-stranded RNA (dsRNA) TLR3 ligand poly-ICLC are two of the most promising vaccine adjuvants currently in clinical development. In preclinical studies, poly-ICLC appears to be the most potent TLR adjuvant when compared to LPS and CpG. This appears due to its induction of pro-inflammatory cytokines and lack of stimulation of IL-10, as well as maintenance of high levels of co-stimulatory molecules in DCs. Poly-ICLC is a synthetically prepared double-stranded RNA consisting of polyl and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and mixed production of type I interferons, cytokines, and chemokines.

Examples of freeze-drying stabilizer may be for example carbohydrates such as sorbitol, mannitol, starch, sucrose, dextran or glucose, proteins such as albumin or casein, and derivatives thereof.

In aspects, Tregitope compounds and compositions of the present disclosure are formulated to be compatible with its intended route of administration. The Tregitope compounds and compositions of the present disclosure can be administered by parenteral, topical, intravenous, oral, subcutaneous, intra-arterial, intradermal, transdermal, rectal, intracranial, intrathecal, intraperitoneal, intranasal; vaginally; intramuscular route or as inhalants. In aspects, Tregitope compound or compositions of the present disclosure can be injected directly into a particular tissue where deposits have accumulated, e.g., intracranial injection. In other aspects, intramuscular injection or intravenous infusion may be used for administration of Tregitope compounds and compositions of the present disclosure. In some methods, T-cell epitope compounds and compositions of the present disclosure are administered as a sustained release composition or device, such as but not limited to a Medipad™ device. In aspects, T-cell epitope compounds and compositions of the present disclosure are administered intradermally, e.g., by using a commercial needle-free high-pressure device such as Pulse NeedleFree technology (Pulse 50™ Micro Dose Injection System, Pulse NeedleFree Systems; Lenexa, KS, USA). In aspects, said commercial needle-free high-pressure device (e.g., Pulse NeedleFree technology) confers one or more of the following benefits: non-invasive, reduces tissue trauma, reduces pain, requires a smaller opening in the dermal layer to deposit the composition in the subject (e.g., only requires a micro skin opening), instant dispersion of the composition, better absorption of the composition, greater dermal exposure to the composition, and/or reduced risk of sharps injury.

In aspects, Tregitope compounds and compositions of the present disclosure can optionally be administered in combination with other agents that are at least partly effective in treating various medical conditions as described herein. For example, in the case of administration into the central nervous system of a subject, Tregitope compound or compositions of the present disclosure can also be administered in conjunction with other agents that increase passage of the agents of the invention across the blood-brain barrier.

In aspects, solutions or suspensions used for parenteral, intradermal, or subcutaneous application can include, but are not limited to, the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents.

In aspects, the parenteral preparation can be enclosed in ampoules, disposable syringes or multiple dose vials made of glass or plastic.

In aspects, pharmaceutical compositions or formulations suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition is sterile and should be fluid to the extent that easy syringeability exists. It is stable under the conditions of manufacture and storage and is preserved against the contaminating action of microorganisms such as bacteria and fungi. In aspects, formulations including a Tregitope compound and composition of the present disclosure may include aggregates, fragments, breakdown products and post-translational modifications, to the extent these impurities bind HLA and present the same TCR face to cognate T cells they are expected to function in a similar fashion to pure Tregitopes. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, and sodium chloride in the composition. Prolonged absorption of the injectable compositions can be brought about by including in the composition a compound that delays absorption, e.g., aluminum monostearate and gelatin.

In aspects, sterile injectable solutions can be prepared by incorporating the Tregitope compounds and compositions of the present disclosure in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Further, Tregitope compounds and compositions of the present disclosure can be administered in the form of a depot injection or implant preparation that can be formulated in such a manner as to permit a sustained or pulsatile release of the active ingredient.

In aspects, oral compositions generally include an inert diluent or an edible carrier and can be enclosed in gelatin capsules or compressed into tablets. In aspects, for the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. In aspects, the tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel or corn starch; a lubricant such as magnesium stearate or Sterotes; a glidant such as colloidal silicon dioxide; a sweetening compound such as sucrose or saccharin; or a flavoring compound such as peppermint, methyl salicylate or orange flavoring.

For administration by inhalation, Tregitope compounds and compositions of the present disclosure can be delivered in the form of an aerosol spray from pressured container or dispenser that contains a suitable propellant, e.g., a gas such as carbon dioxide, or a nebulizer.

In aspects, systemic administration of the Tregitope compounds and compositions of the present disclosure can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the Tregitope compounds and compositions may be formulated into ointments, salves, gels, or creams, and applied either topically or through transdermal patch technology as generally known in the art.

In aspects, the Tregitope compounds and compositions of the present disclosure can also be prepared in the form of suppositories (e.g., with conventional suppository bases such as cocoa butter and other glycerides) or retention enemas for rectal delivery.

In aspects, the Tregitope compounds and compositions of the present disclosure are prepared with carriers that protect the Tregitope compounds and compositions against rapid elimination from the body, such as a controlled-release formulation, including implants and microencapsulated delivery systems. Biodegradable, biocompatible polymers can be used, such as, for example, ethylene vinyl acetate, polyanhydrides, polyglycolic acid, collagen, polyorthoesters, and polylactic acid. Methods for preparation of such formulations will be apparent to those skilled in the art. The materials can also be obtained commercially, e.g., from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art (U.S. Pat. No. 4,522,811, which is herein incorporated by reference in its entirety). In aspects, the Tregitope compounds and compositions of the present disclosure can be implanted within or linked to a biopolymer solid support that allows for the slow release of the Tregitope compounds and compositions to the desired site.

In aspects, it is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the instant disclosure are dictated by and directly dependent on the unique characteristics of the binding agent and the particular therapeutic effect to be achieved, and the limitations inherent in the art of compounding such Tregitope compounds and compositions for the treatment of a subject.

In aspects, the one or more of the Tregitope compounds and compositions as disclosed herein can also be administered to the patient by ex vivo pulsing of isolated dendritic cells (DC) with Tregitopes, followed by reinfusion of the pulsed cells into the patient. These can be prepared according to methods known to those skilled in the art (Butterfield, (2013), Front Immunol, 4:454 and Dissanayake et al., (2014), PLoS One, 9(3)1-10). These reinfusions may be administered by the above methods and compositions.

In aspects of a pharmaceutical composition as described herein, the composition may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the instantly-disclosed peptides or polypeptides (including concatemeric polypeptides) or nucleic acids encoding such peptides or polypeptides (including concatemeric polypeptides). For example, in aspects, a pharmaceutical composition can comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 peptides or polypeptides (including up to 40 peptides or polypeptides), including any value or range therebetween, comprising, consisting of, or consisting essentially of one or more peptides or polypeptides having an amino acid sequence comprising, consisting of, or consisting essentially of one or more of SEQ ID NOS: 1-73 and/or fragments and variants thereof, and optionally 1 to 12 additional amino acids distributed in any ratio on the N terminus and/or C-terminus of the polypeptide of SEQ ID NOS: 1-73; concatemeric peptides as disclosed herein; and nucleic acids (e.g., RNA mRNA, DNA, cDNA) encoding such peptides, polypeptides, or concatemeric peptides, and/or fragments and variants thereof, as described herein.

In aspects, the Tregitope compounds and compositions of the present disclosure (including one or more of e.g., polypeptides (which may be termed herein as “T_{reg} activating regulatory T-cell epitope”, “Tregitope”, or “T-cell epitope polypeptide”) having a sequence comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) are combined in admixture with an antigen, allergen, or a therapeutic protein. Such compositions are useful in methods of inducing tolerance to the antigen, allergen, or a therapeutic protein in a subject in need thereof, wherein local delivery of the admixture with an antigen, allergen, or a therapeutic protein results in increased tolerance to the antigen or allergen in the subject, and delivered with an appropriate excipient resulting in induced tolerance to the antigen, allergen, or a therapeutic protein. This combination may be administered with the Tregitope compounds and compositions of the present disclosure bound either covalently or non-covalently, or they may be administered as an admixture, or a branched or chemically-link preparation. Such compositions are useful in methods of inducing tolerance to an antigen or allergen or a therapeutic protein (e.g., but not limited to, rhGAA). For example, such composition are useful in a subject in need thereof, wherein local delivery of the admixture with an antigen or allergen or therapeutic protein results in increased tolerance to the antigen or allergen or therapeutic protein in the subject, and delivered with an appropriate excipient resulting in induced tolerance to the antigen or allergen or therapeutic protein. In aspects, the Tregitope compounds and compositions of the present disclosure are in combination with a therapeutic blood clotting protein for the purpose of suppressing an immune response against the therapeutic blood clotting protein in a T-cell dependent manner. This combination may be administered with the Tregitope compounds and compositions of the present disclosure bound either covalently or non-covalently, or they may be administered as an admixture. Such compositions are useful in methods of inducing tolerance to the therapeutic blood clotting protein in a subject in need thereof, wherein local delivery of the admixture with the therapeutic blood clotting protein results in increased tolerance to the therapeutic blood clotting protein in the subject, and delivered with an appropriate excipient resulting in induced tolerance to the therapeutic blood clotting protein. In aspects of the above Tregitope compounds and compositions combined in admixture with an antigen or allergen or a therapeutic protein or bound either covalently or non-covalently to with an antigen or allergen or a therapeutic protein, the one or more polypeptides (Treg activating regulatory T-cell epitope, Tregitope, Tregitope peptide, or T-cell epitope polypeptide) of the present disclosure included therein have a sequence: comprising, consisting of, or consisting essentially of one or more of SEQ ID NOS: 1-73.

### Methods of Use

Stimulating regulatory T cells with Tregitope compounds and compositions of the present disclosure (including one or more of e.g., polypeptides (which may be termed herein as “T_{reg} activating regulatory T-cell epitope”, “Tregitope”, or “T-cell epitope polypeptide”) having a sequence comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) can stimulate, induce, and/or expand corresponding naturally occurring T_{Reg} populations (in aspects, including natural T_{Regs} and/or adaptive T_{Reg}s) and in aspects results in increased secretion of one or more of the following cytokines and chemokines: IL-10, IL-35, TGF-β, TNF-α and MCP1. In aspects, stimulation can result in the increased expression of IL-2Rα by corresponding naturally occurring T_{Reg} populations (in aspects, including natural T_{Reg}s and/or adaptive T_{Reg}s) and deprivation of IL-2 to effector T cells. In further aspects, stimulation can result in increased perforin granzyme by corresponding naturally occurring T_{Reg} populations (in aspects, including natural T_{Reg}s and/or adaptive T_{Reg}s), which allows for such Treg populations to kill T effector cells and other immune stimulatory cells. In even further aspects, such stimulation can result in the generation of immune suppressive adenosine by corresponding naturally occurring T_{Reg} populations (in aspects, including natural T_{Reg}s and/or adaptive T_{Reg}s). In other aspects, such stimulation can result in corresponding naturally occurring T_{Reg} populations (in aspects, including natural T_{Regs} and/or adaptive T_{Reg}s) binding to and removing costimulatory molecules on dendritic cells, resulting the inhibition of dendritic cell function. Further, in aspects, such stimulation can result in T_{Reg} induced upregulation of checkpoint molecules on dendritic cells and other cell populations, e.g. but not limited to endothelial cells, by corresponding naturally occurring T_{Reg} populations (in aspects, including natural T_{Reg}s and/or adaptive T_{Reg}s). In additional aspects, such stimulation can result in T_{reg} stimulation of B-regulatory cells. B-regulatory cells (“B-regs”) are cells that are responsible for the anti-inflammatory effect, that is characterized by the expression of CD1d, CD5, and the secretion of IL-10. B-regs are also identified by expression of Tim-1 and can be induced through Tim-1 ligation to promote tolerance. The ability of being B-regs was shown to be driven by many stimulatory factors such as toll-like receptors, CD40-ligand and others. However, full characterization of B-regs is ongoing. B-regs also express high levels of CD25, CD86, and TGF-β. The increased secretion of such regulatory cytokines and chemokines by regulatory T cells, as well as other activities described above, are hallmarks of regulatory T cells. In aspects, regulatory T cells activated by the Tregitope compounds and compositions of the present disclosure may express a CD4+CD25+FOXP3 phenotype. Regulatory T cells activated by the Tregitope compounds and compositions of the present disclosure directly suppress T-effector immune responses ex vivo as measured by decreased antigen-specific Th1- or Th2-associated cytokine levels, principally INF-γ, IL-4, and IL-5, and by decreased proliferation and/or effector function of antigen-specific T effector cells as measured by CFSE dilution and/or cytolytic activity. In aspects, regulatory T cells activated by the Tregitope compounds and compositions of the present disclosure directly suppress T effector immune responses in vivo as measured by decreased antigen-specific Th1- or Th2-associated cytokine levels (as measured by Elisa assay), decreased antigen-specific T effector cell levels (as measured by EliSpot assay), decreased cytolytic activity, and/or decreased antibody titers for protein antigens.

In aspects, natural regulatory T cells activated by the Tregitope compounds and compositions of the present disclosure stimulate the development of adaptive T_{Reg} cells. In aspects, co-incubating peripheral T cells with the Tregitope compounds and compositions of the present disclosure in the presence of antigen results in the expansion of antigen-specific CD4⁺/CD25+ T cells, upregulates the expression of the Foxp3 gene or Foxp3 protein in those cells and suppresses the activation of antigen-specific T effector cells in vitro. In aspects, the Tregitope compounds and compositions of the present disclosure may result in the activation and/or expansion of T regulatory type 1 (Tr1) cells. Tr1 cells have strong immunosuppressive capacity in several immune-mediated diseases (Roncarolo and Battaglia, 2007, Nat Rev Immunol 7, 585-598; Roncarolo et al., 2011, Immunol Rev 241, 145-163; Pot et al., 2011, Semin Immunol 23, 202-208). The secretion of high levels of IL-10, and the killing of myeloid antigen-presenting cells (APCs) via Granzyme B are the main mechanisms of Tr1-mediated suppression (Groux et al., 1997, Nature 389, 737-742; Magnani et al., 2011 Eur J Immunol 41, 1652-1662). Tr1 cells are distinguished from T helper (T_{H})1, T_{H}2, and T_{H}17 cells by their unique cytokine profile and the regulatory function. Tr1 cells have been shown secrete higher levels of IL-10 than IL-4 and IL-17, the hallmark cytokines of T_{H}2 and T_{H}17 cells, respectively. Tr1 cells can also secrete low levels of IL-2 and, depending on the local cytokine milieu, can produce variable levels of IFN-γ, together, the key T_{H}1 cytokines (Roncarolo et al., 2011, Immunol Rev 241, 145-163). FOXP3 is not a biomarker for Tr1 cells since its expression is low and transient upon activation. IL-10-producing Tr1 cells express ICOS (Haringer et al., 2009, J Exp Med 206, 1009-1017) and PD-1 (Akdis et al., 2004, J Exp Med 199, 1567-1575), but these markers are not specific (Maynard et al., 2007, Nat Immunol 8, 931-941). CD49b, the α2 integrin subunit of the very-late-activation antigen (VLA)-2, has been proposed as a marker for IL-10-producing T cells (Charbonnier et al., 2006, J Immunol 177, 3806-3813); but it is also expressed by human T_{H}17 cells (Boisvert et al., 2010, Eur J Immunol 40, 2710-2719). Moreover, murine CD49b⁺ T cells secrete IL-10 (Charbonnier et al., 2006, J Immunol 177, 3806-3813) but also pro-inflammatory cytokines (Kassiotis et al., 2006, J Immunol 177, 968-975). Lymphocyte activation gene-3 (LAG-3), a CD4 homolog that binds with high affinity to MHC class II molecules, is expressed by murine IL-10-producing CD4⁺ T cells (Okamura et al., 2009, Proc Natl Acad Sci USA 106, 13974-13979), but also by activated effector T cells (Workman and Vignali, 2005, J Immunol 174, 688-695; Bettini et al., 2011, J Immunol 187, 3493-3498; Bruniquel et al., 1998, Immunogenetics 48, 116-124; Lee et al., 2012, Nat Immunol 13, 991-999) and by FOXP3+ regulatory T cells (Tregs) (Camisaschi et al., 2010, J Immunol 184, 6545-6551). It was recently shown that human Tr1 cells express CD226 (DNAM-1), which is involved in the specific killing of myeloid APCs (Magnani et al., 2011 Eur J Immunol 41, 1652-1662). In further aspects, Tregitope compounds and compositions of the present disclosure may result in the activation and/or expansion of TGF-β secreting Th3 cells, regulatory NKT cells, regulatory CD8⁺ T cells, double negative regulatory T cells. “Th3 cells” refer to cells having the following phenotype CD4⁺FoxP3⁺ and capable of secreting high levels TGF-β upon activation, amounts of IL-4 and IL-10 and no IFN-γ or IL-2. These cells are TGF-β derived. “Regulatory NKT cells” refers to cells having the following phenotype at rest CD161+CD56+CD16+ and a Vα24/Vβ11 TCR. “Regulatory CD8+ T cells” refers to cells having the following phenotype at rest CD8⁺CD122⁺ and capable of secreting highs levels of IL-10 upon activation. “Double negative regulatory T cells” refers to cells having the following phenotype at rest TCRαβ⁺CD4⁻CD8⁻.

In aspects, the Tregitope compounds and compositions of the present disclosure are useful for regulating immune response to monoclonal antibodies, protein therapeutics, self-antigens promoting autoimmune response, allergens, transplanted tissues and in other applications where tolerance is the desired outcome. In aspects, the Tregitope compounds and compositions of the present disclosure are useful for regulating an immune response caused by rhGAA ERT used to treat patients suffering from Pompe disease. For example, rhGAA can be administered with the Tregitope compounds and compositions of the present disclosure, e.g. but not limited to, through fusion, bonding, or admixture of the rhGAA with the Tregitope compounds and compositions of the present disclosure, or insertion and/or linkage of the Tregitope(s) of the invention into the rhGAA sequence, with the Tregitope compounds and compositions of the present disclosure suppressing an immune response targeting the rhGAA. Such immune reprogramming could reduce or eliminate immune response targeting the rhGAA ERT used to treat Pompe disease, while leaving the balance of the immune system intact.

In aspects, the Tregitopes of the present disclosure can bind MHC class II molecules, engage TCR in context of MHC class II molecules and activate naturally occurring T_{Regs} (in aspects, including natural T_{Regs} and/or adaptive T_{Regs}).

Suppressing an Immune Response in a Subject in Need Thereof. In aspects, the present disclosure is directed to a method of stimulating, inducing, and/or expanding regulatory T-cells (in aspects, naturally occurring T_{Regs}, including natural T_{Regs} and/or adaptive T_{Regs}) in a subject in need thereof and/or suppressing an immune response in a subject in need thereof by administering to the subject a therapeutically effect amount of a Tregitope compound or composition (including one or more of e.g., polypeptides (which may be termed herein as “T_{reg} activating regulatory T-cell epitope”, “Tregitope”, or “T-cell epitope polypeptide”) having a sequence comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) as disclosed herein; nucleic acids, expression cassettes, plasmids, expression vectors, recombinant viruses, or cells (all of which in aspects may be isolated, synthetic, or recombinant) as disclosed herein; chimeric or fusion polypeptide compositions as disclosed herein (which in aspects may be isolated, synthetic, or recombinant); and/or pharmaceutical compositions or formulations as disclosed herein) of the present disclosure.

In aspects, the present disclosure is directed to a method of stimulating/inducing, regulatory T-cells (e.g., naturally occurring T_{Regs} (in aspects, including natural T_{Regs} and/or adaptive T_{Regs})) to suppress an immune response in a subject in need thereof by administering to the subject a therapeutically effective amount of a Tregitope compound or composition of the present disclosure. In aspects, the immune response is the result of one or more therapeutic treatments with at least one therapeutic protein, treatment with a vaccine (particularly in situations in which an adverse event results from the vaccination), or treatment with at least one antigen. In a particular embodiment, the immune response is the result of one or more therapeutic treatments with a rhGAA. Thus, the administration of one or more Tregitope compounds and compositions of the present disclosure can be used to prevent the development of, or terminate, and already-established immune response to establish tolerance induction to rhGAA in patients suffering from Pompe disease. In aspects, the instant disclosure provides methods of using a Tregitope compound or composition of the present disclosure in combination with a therapeutic lysozymal enzyme (e.g., rhGAA) for the purpose of suppressing an immune response against the lysozymal enzyme in a T-cell dependent manner. This combination may be administered with the Tregitope compounds and compositions of the present disclosure bound either covalently or non-covalently, or they may be administered as an admixture. In another aspect, the administration of a Tregitope compound or composition of the present disclosure shifts one or more antigen presenting cells to a regulatory phenotype, one or more dendritic cells to a regulatory phenotype, decreases CD11c and HLA-DR expression in the dendritic cells or other antigen presenting cells.

In aspects, the present disclosure is directed to a method for repressing/suppressing an immune response in a subject, comprising administering a therapeutically effective amount of Tregitope compound or composition of the present disclosure, wherein the Tregitope compound or composition represses/suppresses the immune response. In aspects, the Tregitope compound or composition represses/suppresses an innate immune response. In aspects, the Tregitope compound or composition represses/suppresses an adaptive immune response. In aspects, the Tregitope compound or composition represses/suppresses an effector T cell response. In aspects, the Tregitope compound or composition represses/suppresses a memory T cell response. In aspects, the Tregitope compound or composition represses/suppresses helper T cell response. In aspects, the Tregitope compound or composition represses/suppresses B cell response. In aspects, the Tregitope compound or composition represses/suppresses a nkT cell response.

In aspects, the present disclosure is directed to a method of suppressing an immune response, specifically an antigen specific immune response in a subject, through the administration of a therapeutically effective amount of a Tregitope compound or composition of the present disclosure, wherein said Tregitope compound or composition activates naturally occurring T_{Regs} (in aspects, including natural T_{Regs} and/or adaptive T_{Regs}, and in aspects CD4⁺/CD25⁺/FoxP3+ regulatory T-cells) or suppresses the activation of CD4⁺ T-cells, the proliferation of CD4⁺ and/or CD8⁺ T-cells, and/or suppresses the activation or proliferation of β-cells or nkT Cells. In aspects, a Tregitope compound or composition of the present disclosure may be either covalently bound, non-covalently bound, or in admixture with a specific target antigen. In particular aspects, one or more of e.g., isolated, synthetic, or recombinant isolated, synthetic, or recombinant polypeptides (Treg activating regulatory T-cell epitope, Tregitope, Tregitope peptide, or T-cell epitope polypeptide) and/or chimeric or fusion polypeptide compositions of the presently disclosed Tregitope compounds and compositions may be either covalently bound, non-covalently bound, or in admixture with a specific target antigen. In aspects, an administered Tregitope compound or composition of the present disclosure that is covalently bound, non-covalently bound, or in admixture with a specific target antigen results in the diminution of immune response against the target antigen.

In aspects, the target antigen may be an autologous protein or protein fragment. In aspects, the target antigen may be an allergen. In aspects, the target antigen may be an allogenic protein or protein fragments. In aspects, the target antigen may be a biologic medicine or fragments thereof. In aspects, the target antigen is a coagulation Factor VIII supplement. In aspects, the suppressive effect is mediated by natural T_{Regs}. In aspects, the suppressive effect is mediated by adaptive T_{Regs}. In aspects, the one or more Tregitopes included in the Tregitope compound or composition of the present disclosure suppresses an effector T cell response. In aspects, the one or more Tregitopes of the presently-disclosed Tregitope compound or composition suppresses an innate immune response. In aspects, the one or more Tregitopes of the presently-disclosed Tregitope compound or composition suppresses an adaptive immune response. In aspects, the one or more Tregitopes of the presently-disclosed Tregitope compound or composition suppresses helper T cell response. In aspects, the one or more Tregitopes of the presently-disclosed Tregitope compound or composition suppresses a memory T cell response. In aspects, the one or more Tregitopes of the presently-disclosed Tregitope compound or composition suppresses p cell response. In aspects, the one or more Tregitopes of the presently-disclosed Tregitope compound or composition suppresses nkT cell response.

Designing Small Molecule Therapeutics. In one aspect, the present disclosure provides methods of using a Tregitope compound or composition of the present disclosure for the purpose of designing small molecule therapeutics. In one aspect, Tregitope-specific T cells are stimulated three times with pools of small molecule mixtures at a concentration of 1 μg/ml and autologous dendritic cells (DC) at 2-week intervals, followed by stimulation with heterologous DC and antigens. T cells (1.25×10⁵) and DC (0.25×10⁵) are added per well in round-bottom, 96-well plates. T cell medium is made by supplementing 500 ml of RPMI medium 1640 with 50 ml of FCS (HyClone Laboratories, Inc., Logan, UT), penicillin, and streptomycin (GIBCO Laboratories, Gaithersburg, MD); 20 mM Hepes (GIBCO); and 4 ml 1N NaOH solution. The IL-2 concentration is initially 0.1 nM and gradually is increased to 1 nM during subsequent rounds of stimulation. T cell clones are derived by limiting dilution by using 0.6×10⁵ Epstein-Barr virus-transformed B cells (100 Gray) and 1.3×10⁵ heterologous peripheral blood mononuclear cells (33 Gray) as feeder cells and 1 μg/ml Difco™ phytohemagglutinin (Bacterius Ltd, Houston, TX) in medium containing 2 nM IL-2. Small molecules pools that stimulate the Tregitope specific T cells are then tested as individual molecules.

Cloning T Cell Receptors. In aspects, the present disclosure provides methods of using a Tregitope compound or composition of the present disclosure for the purpose of cloning T cell receptors. Cloning of Tregitope specific T cells can be conducted by techniques known to one of skill in the art. For example, isolated PBMCs are stimulated with Tregitopes at 10 μg/ml RPMI media containing 20% HSA. IL-2 is added (10 U/ml final concentration) every other day starting on day 5. T cells are stained with tetramer pools on day 11 or 12. For each pool, 2-3×10⁵ cells are incubated with 0.5 mg of PE-labeled tetramer in 50 ml of culture medium (10 mg/ml) at 37° C. for 1 to 2 h, and then stained with anti-CD4-FITC (BD PharMingen, San Diego, CA) for 15 min at room temperature. Cells are washed and analyzed with a Becton Dickinson FACSCalibur flow cytometer (Becton Dickinson, San Jose, CA). Tetramers loaded with the corresponding single peptides are generated for those pools that give positive staining, and analysis is done on day 14 or 15. Cells that are positive for a particular tetramer are single-cell sorted into 96-well U-bottom plates by using a Becton Dickinson FACSVantage (San Jose, CA) on the same or following day. Sorted cells are expanded with 1.5-3×10⁵ unmatched, irradiated (5000 rad) PBMC per well as feeders with 2.5 mg/ml PHA and 10 U/ml IL-2 added 24 h later. Specificity of cloned T cells is confirmed by staining with tetramers (loaded with cognate peptide or control peptide, HA307-319) and T cell proliferation assays with 10 mg/ml of specific peptide (Novak E J et al., J Immunol, 166(11):6665-70, which is herein incorporated by reference in its entirety). In aspects, total RNA is extracted with an RNeasy Mini Kit (Qiagene, Hilden, DE) from the Tregitope specific T cell lines generated as described above. One microgram of total RNA is used to clone the TCR cDNAs by a rapid amplification of cDNA end (RACE) method using a GeneRacer© kit (Invitrogen, Carlsbad, CA). Before synthesizing the single-strand cDNA, the RNA is de-phosphorylated, de-capped, and ligated with an RNA oligonucleotide according to the instruction manual of 5′ RACE GeneRacer® kit. SuperScript II RT® (Life Technologies Corp, Carlebad, CA) and GeneRacer® Oligo-dT are used for reverse transcription of the RNA Oligo-ligated mRNA to single-strand cDNAs. 5′ RACE is performed by using GeneRacer® 5′ (GeneRacer® Kit) as 5′ primer and gene-specific primer TCRCAR (5′-GTT AAC TAG TTC AGC TGG ACC ACA GCC GCA GC-3′; SEQ ID NO: 107) or TCRCB1R (5′-CGG GTT AAC TAG TTC AGA AAT CCT TTC TCT TGA CCA TGG C-3′; SEQ ID NO: 108), or TCRCBR2 (5′-CTA GCC TCT GGA ATC CTT TCT CTT G-3′; SEQ ID NO: 109) as 3′ primers for TCR α, μ1, or β2 chains, respectively. The polymerase chain reaction (PCR) products are cloned into pCR2.1 TOPO vector (Invitrogen, Carlsbad, CA) and then transformed into One Shot TOP10 Competent Escherichia coli (Invitrogen, Carlsbad, CA). Plasmid DNAs are prepared from 96 individual clones from each construct for TCRα, β1, and β2 chains. Full-length insert of all the plasmids is sequenced to determine the vα/vβ usage (Zhao Y et al., (2006), J Immunother, 29(4):398-406, herein incorporated by reference in its entirety).

Methods of Preventing or Treating a Medical Condition. The present disclosure is directed to, for example methods of preventing or treating one or more medical conditions in a subject comprising administering a Tregitope compound or composition of the present disclosure, and preventing or treating the medical condition in a subject by said step of administering. The medical condition can be, for example, primary immunodeficiencies (such as autoimmunity associated with primary immune deficiency disorders); immune-mediated thrombocytopenia, Kawasaki disease, hematopoietic stem cell transplantation in patients older than 20 years, chronic B-cell lymphocytic leukemia and pediatric HIV type 1 infections. Specific examples include: (Hematology) aplastic anemia, pure red cell aplasia, Diamond-Blackfan anemia, autoimmune hemolytic anemia, hemolytic disease of the newborn, acquired factor VIII inhibitors, acquired von Willebrand disease, immune-mediated neutropenia, refractoriness to platelet transfusion, neonatal alloimmune/autoimmune thrombocytopenia, posttransfusion purpura, thrombotic thrombocytopenia purpura/hemolytic uremic syndrome; (Infectious diseases), solid organ transplantation, surgery, trauma, burns, and HIV infection; (Neurology) epilepsy and pediatric intractable Guillain-Barre syndrome, chronic inflammatory demyelinating polyneuropathy, myasthenia gravis, Lambert-Eaton myasthenic syndrome, multifocal motor neuropathy, multiple sclerosis; (Obstetrics) recurrent pregnancy loss; (Pulmonology) asthma, chronic chest symptoms, rheumatology, rheumatoid arthritis (adult and juvenile), systemic lupus erythematosus, systemic vasculitides, dermatomyositis, polymyositis, inclusion-body myositis, wegener granulomatosis; (lysosomal) Pompe disease (or glycogen storage disease II), Gaucher disease, Fabry disease, Niemann-Pick disease, Hunter syndrome, Tay-Sachs disease; (Miscellaneous) adrenoleukodystrophy, amyotrophic lateral sclerosis, Behçet syndrome, acute cardiomyopathy, chronic fatigue syndrome, congenital heart block, cystic fibrosis, autoimmune blistering dermatosis, diabetes mellitus, acute idiopathic dysautonomia, acute disseminated encephalomyelitis, endotoxemia, hemolytic transfusion reaction, hemophagocytic syndrome, acute lymphoblastic leukemia, lower motor neuron syndrome, multiple myeloma, human T-cell lymphotrophic virus-1-associated myelopathy, nephritic syndrome, membranous nephropathy, nephrotic syndrome, euthyroid ophthalmopathy, opsoclonus-myoclonus, recurrent otitis media, paraneoplastic cerebellar degeneration, paraproteinemic neuropathy, parvovirus infection (general), polyneuropathy, organomegaly, endocrinopathy, M-protein, and skin changes (POEMS) syndrome, progressive lumbosacral plexopathy, lyme radiculoneuritis, Rasmussen syndrome, Reiter syndrome, acute renal failure, thrombocytopenia (nonimmune), streptococcal toxic shock syndrome, uveitis and Vogt-Koyanagi-Harada syndrome.

In a particular aspect, the present disclosure is directed to, for example, methods of treating allergy, autoimmune disease, transplant-related disorders such as graft versus host disease, enzyme or protein deficiency disorders, lysosomal storage disorders (e.g., Pompe disease), cancers (particularly tumor associated autoimmunity), infertility, or infections (viral, bacterial, or parasitic). The Tregitope compound or composition of the present disclosure can be used with in conjunction with other proteins or compounds used for treating a subject with a medical condition in order to reduce adverse events or enhance the efficacy of the co-administered compound.

Application to Preventing or Treating an Antibody and/or CD4+ T cell Response Caused by rhGAA used to treat Patients Suffering from Pompe disease. Pompe is a disease characterized by the absence of the lysosomal enzyme acid alpha-glucosidase (GAA). The lack of the GAA enzyme typically presents itself during the first few weeks of life post-partum. Symptoms of effects caused by the lack of GAA include hypotonia, muscle weakness, marcoglossia, and hypertrophic cardiomyopathy. Untreated, infantile patients are expected to doe within less than a year. Pompe disease is an autosomal recessive genetic disorder, caused by mutation in a gene at 17q25.2-q25.3 that encodes acid alpha-glucosidase (GAA). Typically, the mutation is a transversion of a thymine to guanine and affected individuals carry two defective copies. The absence of functional GAA in an individual prevents the degradation of alpha 1.4 and alpha 1.6 linkages in glycogen, maltose and isomaltose and results in accumulation in lysosomes and cytoplasm that quickly affects other organelles and leads to cellular injury, primarily in skeletal, cardiac and smooth muscle.

The development of a treatment was achieved in 2006 with the approval of alglucosidase alfa (rhGAA, Myozyme, Lumizyme), a recombinant human GAA that could be administered to diagnosed patients. This therapy has changed the typical outcome for many patients, with large numbers now in long-term survival. However, there have been identified several factors that can determine how a patient might respond to rhGAA enzyme replacement therapy (ERT). One such indicator being the CRIM-status of the patient (cross-reactive immunologic material) (see, Kishnani et al., Mol. Genet. Metab. 99:26-33, 2010; Banugaria et al., PLoS One 8: e67052, 2013; Banugaria et al., Genet Med. 113: 729-36, 2011). It has been identified that CRIM-negative patients, which account for about a third of infantile Pompe disease (IPD) patients, exhibit high antibody titers reactive to administered rhGAA. Further still, some CRIM-positive patients can unpredictably develop a high antibody titer against rhGAA. Therapies such as immune tolerance induction (ITI) showed a reduction in antibody titers compared to ERT monotherapy. An ITI protocol using Rituximab, Methotrexate, and IVIG has been successful for the treatment and prevention of ATA, and is the standard of care for all CRIM-negative patients (3,11). However, there is currently no method for determining which CRIM-positive patients are at risk of developing treatment-limiting anti-therapeutic antibodies (ATA). Treating all CRIM-positive patients with ITI may unnecessarily expose approximately two-thirds of CRIM-positive patients who would not develop treatment-limiting ATA to potential long-term adverse effects of ITI therapy.

One strategy both for the prevention and therapy of an unwanted antibody and/or CD4+ T cell response caused by rhGAA used as ERT in patients suffering from Pompe disease is the induction of regulatory T cells. These patients can be protected from developing an unwanted immune response when receiving rhGAA ERT. In aspects, the present invention is directed to preventing or diminishing an autoimmune response caused by rhGAA used to treat Pompe disease patients comprising administering a Tregitope compound or composition of the present disclosure, thereby treating the medical condition. The Tregitope compounds and compositions of the invention can be used with or in conjunction with other proteins or compounds (e.g., but not limited to, human coagulation Factor VIII supplements) used for treating a subject with a medical condition (e.g., but not limited to Hemophilia A) in order to reduce adverse events or enhance the efficacy of the co-administered compound.

Application to Allergy. Allergen-specific regulatory T cells play an important role in controlling the development of allergy and asthma. Naturally occurring T_{Regs} (in aspects, including natural T_{Regs} and/or adaptive T_{Regs}, and in aspects CD4⁺/CD25⁺/FoxP3+ regulatory T-cells) have been shown to inhibit the inappropriate immune responses involved in allergic diseases. A number of recent studies indicate that regulatory T cells play an important role in controlling the overdevelopment of T-helper type 2 biased immune responses in susceptible individuals, not only in animal models, but in humans as well. Recent studies indicate that T_{regs} also suppress T cell co-stimulation by the secretion of TGF-β and IL-10, suggesting an important role of T_{regs} in the regulation of allergic disorders. Impaired expansion of natural or adaptive regulatory T cells leads to the development of allergy, and treatment to induce allergen-specific T_{regs} would provide curative therapies for allergy and asthma. One strategy both for the prevention and therapy of asthma is the induction of T_{regs}. Animals can be protected from developing asthma by immune stimulation leading to Th1 or T_{reg} responses. Accordingly, Tregitope compounds and compositions of the present disclosure are useful in methods for the prevention or treatment of allergy and/or asthma. As such, in aspects, the present disclosure is directed to a method of preventing or treating allergy and/or asthma in a subject, the method comprising administering a therapeutically-effective amount of a Tregitope compound or composition of the present disclosure, and preventing or treating allergy and/or asthma in a subject by said step of administering.

Application to Transplantation. The Tregitope compounds and compositions of the present disclosure are useful to induce tolerance during the transplantation process, by promoting the development of cells that specifically down regulate immune responses against donor cells. Induction of Ag-specific T_{Reg} cells for treating organ-specific autoimmunity is an important therapeutic development, avoiding generalized immune suppression. In murine models of bone marrow transplantation, T_{Regs} promote donor bone marrow engraftment and decrease the incidence and severity of graft versus host disease without abrogating the beneficial graft versus tumor immunologic effect. These findings, in concert with observations that T_{Regs} in mice and humans share phenotypic and functional characteristics, have led to active investigations into the use of these cells to decrease complications associated with human hematopoietic cell transplantation. An imbalance of T_{Reg}s and effector T cells contributes to the development of graft versus host disease, however, the mechanisms of immunoregulation, in particular, the all recognition properties of T_{Reg}s, their effects on and interaction with other immune cells, and their sites of suppressive activity, are not well understood.

Accumulating evidence from both humans and experimental animal models has implicated the involvement of T_{Reg}s in the development of graft versus host disease (GVHD). The demonstration that T_{Reg}s can separate GVHD from graft versus tumor (GVT) activity suggests that their immunosuppressive potential could be manipulated to reduce GVHD without detrimental consequence on GVT effect. Although a variety of T lymphocytes with suppressive capabilities have been reported, the two best-characterized subsets are the naturally arising, intrathymic-generated T_{Reg}s (natural T_{Reg}s) and the peripherally generated, adaptive T_{Regs} (adaptive T_{Reg}s). Accordingly, Tregitope compounds and compositions of the present disclosure are useful in methods for inducing tolerance during the transplantation process. As such, in aspects, the present disclosure is directed to a method of inducing tolerance during the transplantation process in a subject, the method comprising administering a therapeutically-effective amount of a Tregitope compound or composition of the present disclosure, and inducing tolerance during the transplantation process in a subject by said step of administering.

Application as a Tolerizing Agent and to Autoimmunity. In aspects, Tregitope compounds and compositions of the present disclosure can be used as a tolerizing agents for immunogenic compounds (protein therapeutics) (Weber C A et al., (2009), Adv Drug Deliv, 61(11):965-76). This discovery has implications for the design of protein therapeutics. Thus, administration of a monoclonal antibody, autologous cytokine, or foreign protein in conjunction with a Tregitope compound or composition of the present disclosure suppresses adverse T effector immune responses. In vivo, T_{Reg}s act through dendritic cells to limit autoreactive T-cell activation, thus preventing their differentiation and acquisition of effector functions. By limiting the supply of activated pathogenic cells, T_{Reg}s prevent or slow down the progression of autoimmune diseases. This protective mechanism appears, however, insufficient in autoimmune individuals, likely because of a shortage of T_{Reg}s cells and/or the development and accumulation of T_{Reg}-resistant pathogenic T cells over the long disease course. Thus, restoration of self-tolerance in these patients may require purging of pathogenic T cells along with infusion of T_{Reg}s with increased ability to control ongoing tissue injury. Organ-specific autoimmune conditions, such as thyroiditis and insulin-dependent diabetes mellitus have been attributed to a breakdown of this tolerance mechanism (Mudd P A et al., (2006), Scand J Immunol, 64(3):211-8). Accordingly, Tregitope compounds and compositions of the present disclosure are useful in methods for the prevention or treatment of autoimmunity. As such, in aspects, the present disclosure is directed to a method of preventing or treating autoimmunity in a subject, the method comprising administering a therapeutically-effective amount of a Tregitope compound or composition of the present disclosure, and preventing or treating autoimmunity in a subject by said step of administering.

Application to Diabetes. Type 1 (juvenile) diabetes is an organ-specific autoimmune disease resulting from destruction of insulin-producing pancreatic beta-cells. In non-diabetics, islet cell antigen-specific T cells are either deleted in thymic development or are converted to T regulatory cells that actively suppress effector responses to islet cell antigens. In juvenile diabetics and in the NOD mouse model of juvenile diabetes, these tolerance mechanisms are missing. In their absence, islet cell antigens are presented by human leukocyte antigen (HLA) class I and II molecules and are recognized by CD8(+) and CD4(+) auto-reactive T cells. Destruction of islet cells by these auto-reactive cells eventually leads to glucose intolerance. Co-administration of Tregitopes and islet cell antigens leads to the activation of naturally occurring T regulatory cells and the conversion of existing antigen specific effector T cell to a regulatory phenotype. In this way, deleterious autoimmune response is redirected leading to the induction of antigen-specific adaptive tolerance. Modulation of auto-immune responses to autologous epitopes by induction of antigen-specific tolerance can prevent ongoing beta-cell destruction. Accordingly, Tregitope compounds and compositions of the present disclosure are useful in methods for the prevention or treatment of diabetes. As such, in aspects, the present disclosure is directed to a method of preventing or treating diabetes in a subject, the method comprising administering a therapeutically-effective amount of Tregitope compound or composition of the present disclosure, and preventing or treating diabetes in a subject by said step of administering.

Application to Hepatitis B (HBV) infection. Chronic HBV is usually either acquired (by maternal fetal transmission) or can be a rare outcome of acute HBV infection in adults.

Acute exacerbations of chronic hepatitis B (CH—B) are accompanied by increased cytotoxic T cell responses to hepatitis B core and e antigens (HBcAg/HBeAg). In a recent study, the SYFPEITHI T cell epitope mapping system was used to predict MHC class II-restricted epitope peptides from the HBcAg and HbeAg (Feng I C et al., (2007), J Biomed Sci, 14(1):43-57). MHC class II tetramers using the high scoring peptides were constructed and used to measure T_{Reg} and CTL frequencies. The results showed that T_{Reg} cells specific for HBcAg declined during exacerbations accompanied by an increase in HBcAg peptide-specific cytotoxic T cells. During the tolerance phase, FOXp3-expressing T_{Reg} cell clones were identified. These data suggest that the decline of HbcAg T_{Reg} T cells accounts for the spontaneous exacerbations on the natural history of chronic hepatitis B virus infection. Accordingly, Tregitope compounds and compositions of the present disclosure are useful in methods for the prevention or treatment of viral infections. As such, in aspects, the present disclosure is directed to a method of preventing or treating a viral infection (e.g., HBV infection) in a subject, the method comprising administering a therapeutically-effective amount of a Tregitope compound or composition of the present disclosure, and preventing or treating said viral infection in a subject by said step of administering.

Application to SLE. A T_{Reg} epitope that plays a role in Systemic Lupus Erythematosis (SLE) or Sjögren's syndrome has been defined. This peptide encompasses residues 131-151 (RIHMVYSKRSGKPRGYAFIEY; SEQ ID NO: 110) of the spliceosome protein. Binding assays with soluble HLA class II molecules and molecular modeling experiments indicated that the epitope behaves as promiscuous epitope and binds to a large panel of human DR molecules. In contrast to normal T cells and T cells from non-lupus autoimmune patients, PBMCs from 40% of randomly selected lupus patients contain T cells that proliferate in response to peptide 131-151. Alteration of the ligand modified the T cell response, suggesting that several populations of T cells responding to this peptide exist, among which may be T_{Reg} cells. T regulatory epitopes have also been defined in Sjögren's syndrome. Accordingly, Tregitope compounds and compositions of the present disclosure administered in combination with the Tregitope of SEQ ID NO: 110 are useful in methods for the prevention or treatment of SLE. As such, in aspects, the present disclosure is directed to a method of preventing or treating SLE in a subject, the method comprising administering a therapeutically-effective amount of a Tregitope compound or composition of the present disclosure in combination with the Tregitope of SEQ ID NO: 68, and preventing or treating SLE in a subject by said step of administering.

Application to Autoimmune Thyroiditis. Autoimmune Thyroiditis is a condition that occurs when antibodies arise to self-thyroid peroxidase and/or thyroglobulin, which cause the gradual destruction of follicles in the thyroid gland. HLA DR5 is closely associated with the disease. Accordingly, Tregitope compounds and compositions of the present disclosure administered in combination with thyroid peroxidase and/or thyroglobulin TSHR or portions thereof are useful in methods for the prevention or treatment of autoimmune thyroiditis. As such, in aspects, the present disclosure is directed to a method of preventing or treating autoimmune thyroiditis in a subject, the method comprising administering a therapeutically-effective amount of a Tregitope compound or composition of the present disclosure in combination with thyroid peroxidase and/or thyroglobulin TSHR or portions thereof, and preventing or treating autoimmune thyroiditis in a subject by said step of administering. In further aspects, Tregitope compounds and compositions of the present disclosure administered in combination with TSHR or other Graves' disease antigens or portions thereof are useful in methods for the prevention or treatment of Grave's disease. Graves' disease is an autoimmune disorder that is characterized by antibodies to self-thyroid stimulating hormone receptor (TSHR) leading to leading to hyperthyroidism, or an abnormally strong release of hormones from the thyroid gland. Several genetic factors can influence susceptibility to Graves' disease. Females are much more likely to contract the disease than males; White and Asian populations are at higher risk than black populations and HLA DRB1-0301 is closely associated with the disease. As such, in aspects, the present disclosure is directed to a method of preventing or treating Grave's disease in a subject, the method comprising administering a therapeutically-effective amount of a Tregitope compound or composition of the present disclosure in combination with TSHR or other Graves' disease antigens or portions thereof, and preventing or treating Grave's disease in a subject by said step of administering.

Ex Vivo Expansion and/or Stimulation of T-Regulatory Cells Using Tregitope compounds and compositions. In aspects, the present disclosure provides ex vivo methods for the expansion of regulatory T-cells. In one embodiment, the invention provides a method of expanding regulatory T-cells in a biological sample, the method comprising: (a) providing a biological sample from a subject; (b) isolating regulatory T-cells from the biological sample; and contacting the isolated regulatory T-cells with an effective amount of a Tregitope compound or composition of the present disclosure under conditions wherein the T-regulatory cells increase in number to yield an expanded regulatory T-cells, thereby expanding the regulatory T-cells in the biological sample. In aspects, the method further comprises the step of administration of the expanded regulatory T-cells to a subject. In aspects, the subject administered the expanded regulatory T-cells is the same individual from which the original biological sample was obtained, e.g., by autologous transplantation of the expanded Tregitope (Ruitenberg J J et al., (2006), BMC Immunol, 7:11).

In aspects, the present disclosure provides ex vivo methods for stimulation of regulatory T-cells in a biological sample, the method comprising: (a) providing a biological sample from a subject; (b) isolating regulatory T-cells from the biological sample; and contacting the isolated regulatory T-cells with an effective amount of a Tregitope compound or composition of the present disclosure under conditions wherein the T-regulatory cells are stimulated to alter one or more biological function, thereby stimulating the regulatory T-cells in the biological sample. In aspects, the method further comprises the step of administration of the stimulated regulatory T-cells to a subject. In aspects, the subject administered the stimulated regulatory T-cells is the same individual from which the original biological sample was obtained, e.g., by autologous transplantation of the expanded Tregitope.

Ex Vivo Pulsing of Antigen Presenting Cells using Tregitope compounds and compositions. In aspects, the present disclosure provides ex vivo methods for antigen presenting cells (e.g., dendritic cells, macrophages, etc.) in a biological sample, the method comprising: (a) providing a biological sample from a subject; (b) isolating antigen presenting cells from the biological sample; and contacting the isolated antigen presenting with an effective amount of a Tregitope compound or composition of the present disclosure under conditions wherein the antigen presenting cells are stimulated to alter one or more biological function (e.g., to present the Tregitopes and/or skew the antigen presenting cells to a be tolerogenic (which in aspects can further include cytokine treatment of the antigen presenting cells to induce such a tolerogenic state), thereby stimulating the antigen presenting cells in the biological sample. In aspects, the method further comprises the step of administration of the stimulated antigen presenting cells to a subject. In aspects, the subject administered the stimulated antigen presenting cells is the same individual from which the original biological sample was obtained, e.g., by autologous transplantation of the stimulated antigen presenting cells.

In Vitro Uses of Tregitope compounds and compositions. In aspects, the present disclosure provides the use of a Tregitope compound or composition of the present disclosure as reagents in the study of regulatory T-cell function in in vitro studies and experimental models.

Methods of Immune Engineering. In aspects, the present disclosure is directed to a methods of immune engineering, including removal or insertion of one or more Tregitopes of the instant disclosure, from or into a polypeptide.

For example, in aspects the present disclosure is directed to a method for decreasing the immunogenicity and/or increasing tolerogenicity of a polypeptide, which may be particularly useful when a polypeptide (such as GAA/LYAG or a recombinant GAA, such as a recombinant human GAA (“rhGAA” or alglucosidase alfa) supplement, (or fragments thereof)) serves as a therapeutic protein. In aspects, said method comprises insertion of one or more regulatory T cell epitopes (e.g., a peptide or polypeptide comprising, consisting of, or consisting essentially of one or more peptides or polypeptides having an amino acid sequence of SEQ ID NOS: 1-73 (and/or fragments or variants thereof), and optionally 1 to 12 additional amino acids distributed in any ratio on the N-terminus and/or C-terminus of the polypeptide of SEQ ID NOS: 1-73) into said polypeptide (e.g., GAA/LYAG or a recombinant GAA supplement (or fragments thereof). In aspects, the one or more regulatory T cell epitopes inserted into the GAA/LYAG or a recombinant GAA supplement (or fragments thereof) can suppress an antigen-specific immune response against the human GAA/LYAG molecule or replacement protein/supplement (or fragments thereof). In aspects, said one or more regulatory T cell epitopes may be fused to or inserted internally within (e.g., but not limited to, site directed mutagenesis or other recombinant techniques) a human GAA/LYAG or a recombinant GAA supplement (or fragments thereof), such as in instances where the Tregitope is not located in its natural position within the GAA/LYAG molecule or replacement protein/supplement (or fragments thereof) or wherein the GAA/LYAG molecule or replacement protein/supplement (or fragments thereof) thereof is missing such a Tregitope (e.g., if a particular GAA/LYAG molecule or replacement protein/supplement (or fragments thereof) has a mutated or missing corresponding section). In aspects, said insertion of the one or more regulatory T cell epitopes into the GAG/LYAGmolecule or replacement protein/supplement (or fragments thereof) thereof comprises insertion of all or some of the amino acids of the one or more regulatory T cell epitopes. In aspects, said insertion of the one or more regulatory T cell epitopes into the GAG/LYAG molecule or replacement protein/supplement (or fragments thereof) thereof comprises insertion of some or all of the amino acids of the one or more regulatory T cell epitopes and removing one or more amino acids at the site of insertion of the regulatory T cell epitope amino acids. In aspects, said insertion of the one or more regulatory T cell epitopes into the GAG/LYAG molecule or replacement protein/supplement (or fragments thereof) comprises mutating the sequence of the GAG/LYAG molecule or replacement protein/supplement (or fragments thereof) thereof to include the one or more regulatory T cell epitopes (for example, but not limited to, introduction one or more point mutations into the antibody or fragment thereof by site-directed mutagenesis or other recombinant techniques). In aspects, said insertion of the one or more regulatory T cell epitopes into the GAG/LYAG molecule or replacement protein/supplement (or fragments thereof), which in aspects will introduce the one or more regulatory T cell epitope sequences, such that the previous immunogenicity of the sequence is decreased and the tolerogenicity of the new sequence is enhanced. In aspects, the number of said added one or more amino acids at the site of insertion of the regulatory T cell epitope amino acids need not correspond to the number of amino acids deleted from the sequence of the GAG/LYAG molecule or replacement protein/supplement (or fragments thereof). In aspects, said insertion of one or more regulatory T cell epitopes into the GAG/LYAG molecule or replacement protein/supplement (or fragments thereof) results in decreasing the immunogenicity of the antibody or fragment thereof. In aspects, said insertion of one or more regulatory T cell epitopes into the GAG/LYAG molecule or replacement protein/supplement (or fragments thereof) thereof results in a increasing the tolerogenicity of the GAG/LYAG molecule or replacement protein/supplement (or fragments thereof). In aspects, the one or more regulatory T cell epitopes have a sequence comprising, consisting of, or consisting essentially of one or more of SEQ ID NOS: 1-73.

Kits. The methods described herein can be performed, e.g., by utilizing pre-packaged kits comprising at least one Tregitope compound or composition of the present disclosure, which can be conveniently used, e.g., in clinical settings to treat subjects exhibiting symptoms or family history of a medical condition described herein. In one embodiment, the kit further comprises instructions for use of the at least one Tregitope compound or composition of the instant disclosure to treat subjects exhibiting symptoms or family history of a medical condition described herein.

### Aspects

A 1^{st} aspect is directed to a polypeptide consisting of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS: 1-73.

A 2^{nd} aspect is directed to a polypeptide consisting essentially of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS: 1-73.

A 3^{rd} aspect is directed to a polypeptide comprising an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS: 1-73.

A 4th aspect is directed to a polypeptide according to any one of aspects 1-3, wherein said variant or fragment of an amino acid sequence selected from the group consisting of SEQ ID NOS: 1-73 retains MHC binding propensity and TCR specificity, and/or retains regulatory T cell stimulating or suppressive activity.

A 5^{th} aspect is directed to a polypeptide consisting of an amino acid sequence having at least 75%, 80%, 85%, 90%, or 95% homology to any one of SEQ ID NOS: 1-73, and fragments thereof, wherein said polypeptide retains MHC binding propensity and the same TCR specificity, and/or retains regulatory T cell stimulating or suppressive activity.

A 6^{th} aspect is directed to a polypeptide consisting essentially of an amino acid sequence having at least 75%, 80%, 85%, 90%, or 95% homology to any one of SEQ ID NOS: 1-73, and fragments thereof, wherein said polypeptide retains MHC binding propensity and the same TCR specificity, and/or retains regulatory T cell stimulating or suppressive activity.

A 7^{th} aspect is directed to a polypeptide comprising an amino acid sequence having at least 75%, 80%, 85%, 90%, or 95% homology to any one of SEQ ID NOS: 1-73, and fragments thereof, wherein said polypeptide retains MHC binding propensity and the same TCR specificity, and/or retains regulatory T cell stimulating or suppressive activity.

An 8^{th} aspect is directed to a polypeptide according to any one of aspects 1-7, wherein said polypeptide has one or more conservative substitutions compared to the polypeptide.

A 9th aspect is directed to a polypeptide according to aspect 8, wherein said polypeptide retains MHC binding propensity and TCR specificity, and/or retains regulatory T cell stimulating or suppressive activity.

A 10^{th} aspect is directed to a polypeptide composition comprising one or more T-cell epitope polypeptides linked to a heterologous polypeptide, wherein the T-cell epitope polypeptide is a polypeptide according to any one of aspects 1-9.

An 11^{th} aspect is directed to a polypeptide composition according to aspect 10, wherein the T-cell epitope polypeptide is linked to the N-terminus of the heterologous polypeptide.

An 12th aspect is directed to a polypeptide composition according to any one or aspects 10-11, wherein the T-cell epitope polypeptide is linked to the C-terminus of the heterologous polypeptide.

A 13^{th} aspect is directed to a polypeptide composition according to any one or aspects 10-12, wherein the heterologous polypeptide comprises a biologically active molecule and wherein the biologically active molecule is selected from the group consisting of an immunogenic molecule, a T-cell epitope, a viral protein, and a bacterial protein.

A 14^{th} aspect is directed to a polypeptide composition according to any one or aspects 10-13, wherein the heterologous polypeptide is operatively linked to the T-cell epitope polypeptide.

A 15^{th} aspect is directed to a nucleic acid encoding a polypeptide consisting of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS: 1-73.

A 16^{th} aspect is directed to a nucleic acid encoding a polypeptide consisting essentially of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS: 1-73.

A 17^{th} aspect is directed to a nucleic acid encoding a polypeptide comprising an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS: 1-73.

A 18^{th} aspect is directed to a nucleic acid of any one of aspects 8-10, wherein said fragment or variant of the nucleic acid encoding a polypeptide comprising an amino acid sequence selected from the group consisting of SEQ ID NOS: 1-73 encodes a polypeptide that retains regulatory T cell stimulating or suppressive activity.

A 19^{th} aspect is directed to a plasmid comprising a nucleic acid of any one of aspects 15-18.

A 20^{th} aspect is directed to a vector comprising a nucleic acid according to any one of aspects 15-18.

A 21^{st} aspect is directed to a pharmaceutical composition comprising a polypeptide according to any one of aspects 1-14 and a pharmaceutically-acceptable carrier and/or excipient.

A 22^{nd} aspect is directed to a pharmaceutical composition comprising a nucleic acid according to any one of aspects 15-18 and a pharmaceutically-acceptable carrier and/or excipient.

A 23^{rd} aspect is directed to a pharmaceutical composition comprising a plasmid according to aspect 19 and a pharmaceutically-acceptable carrier and/or excipient.

A 24^{th} aspect is directed to a pharmaceutical composition comprising a vector according to aspect 20 and a pharmaceutically-acceptable carrier and/or excipient.

A 25^{th} aspect is directed to a method for suppressing an immune response in a subject in need thereof, the method comprising administering to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 26^{th} aspect is directed to a method of inducing regulatory T-cells to suppress immune response in a subject comprising administrating to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 27^{th} aspect is directed to a method for stimulating regulatory T-cells in a subject in need thereof, the method comprising administering to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 28^{th} aspect is directed to a method suppressing an antigen-specific immune response in a subject in need thereof, the method comprising administering to the subject a therapeutically effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24.

A 29^{th} aspect is directed to a method according to any one of aspects 25-28, wherein the administration of the Tregitope composition activates CD4⁺/CD25⁺/FoxP3+ regulatory T-cells.

A 30^{th} aspect is directed to a method according to any one of aspects 25-28, wherein the administration of the Tregitope composition suppresses activation of CD4⁺ T-cells.

A 31^{st} aspect is directed to a method according to any one of aspects 25-28, wherein the administration of the Tregitope composition suppresses activation or proliferation of CD4+ effector T-cells and/or CD8+ effector T-cells.

A 32^{nd} aspect is directed to a method according to any one of aspects 25-28, wherein the administration of the Tregitope composition suppresses activation or proliferation of B-cells.

A 33^{rd} aspect is directed to a method according to any one of aspects 25-28, wherein the subject suffers from an allergy, an autoimmune disease, a transplant related disorder, an enzyme or protein deficiency disorder, or a blood clotting disorder.

A 34^{th} aspect is directed to a method according to any one of aspects 25-28, wherein the immune response is a result of one or more therapeutic treatments select from the group consisting of, treatment with at least one therapeutic protein, treatment with a vaccine, and treatment with at least one antigen

A 35^{th} aspect is directed to a method according to any one of aspects 25-28, wherein the administration of the pharmaceutical Tregitope composition shifts one or more antigen presenting cells to a regulatory phenotype.

A 36^{th} aspect is directed to a method according to any one of aspects 25-28, wherein the administration of the regulatory T-cell epitope shifts one or more dendritic cells to a regulatory phenotype.

A 37^{th} aspect is directed to a method according to any one of aspects 25-28, wherein the administration of the pharmaceutical Tregitope composition shifts one or more dendritic cells to a regulatory phenotype.

A 38^{th} aspect is directed to a method according to any one of aspects 25-28, wherein the regulatory phenotype is characterized by a decrease in CD11c and HLA-DR expression in the dendritic cells or other antigen presenting cells.

A 39^{th} aspect is directed to a method according to any one of aspects 25-28, wherein the administration of the regulatory T-cell epitope shifts one or more T cells to a regulatory phenotype.

A 40^{th} aspect is directed to a method according to any one of aspects 25-28, wherein the administration of the regulatory T-cell epitope shifts one or more CD4⁺ T cells to a regulatory phenotype.

A 41^{st} aspect is directed to a method according to any one of aspects 25-28, wherein the administration of the regulatory T-cell epitope shifts one or more CD8⁺ T cells to a regulatory phenotype.

A 42^{nd} aspect is directed to a method according to any one of aspects 25-28, wherein the administration of the regulatory T-cell epitope shifts one or more B cells to a regulatory phenotype.

A 43^{rd} aspect is directed to a method for expanding a population of regulatory T cells of a patient, comprising:
(a) providing a biological sample obtained from a subject; and
(b) isolating regulatory T-cells from the biological sample; and contacting the isolated regulatory T-cells with an effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24 under conditions wherein the T-regulatory cells increase in number to yield an expanded regulatory T-cell composition, thereby expanding the regulatory T-cells in the biological sample; and
(c) returning said increased number of regulatory T cells to said patient.

A 44^{th} aspect is directed to a method for stimulating regulatory T cells in a biological sample, comprising:
(a) providing a biological sample obtained from a subject;
(b) isolating regulatory T-cells from the biological sample; and contacting the isolated regulatory T-cells with an effective amount of one or more of a polypeptide according to any one of aspects 1-14, a nucleic acid according to any one of aspects 15-18, a plasmid according to aspect 19, a vector according to aspect 20, or a pharmaceutical composition according to any one of aspects 21-24 under conditions wherein the T-regulatory cells are stimulated to alter one or more biological function, thereby stimulating the regulatory T-cells in the biological sample.

### EXEMPLIFICATION

The examples that follow are not to be construed as limiting the scope of the invention in any manner. In light of the present disclosure, numerous embodiments within the scope of the claims will be apparent to those of ordinary skill in the art. While certain examples pertain to particular Tregitopes of the instant disclosure, it will be understood that examples and methods can be used for any Tregitope of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73).

### EXEMPLIFICATION

The examples that follow are not to be construed as limiting the scope of the invention in any manner. In light of the present disclosure, numerous embodiments within the scope of the claims will be apparent to those of ordinary skill in the art. While certain examples pertain to particular Tregitopes of the instant disclosure, it will be understood that examples and methods can be used for any Tregitope of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73).

### (1) In-Silico Identification of a Tregitope Compound or Composition

T cells specifically recognize epitopes presented by antigen presenting cells (APCs) in the context of MHC (Major Histocompatibility Complex) Class II molecules. These T-helper epitopes can be represented as linear sequences comprising 7 to 30 contiguous amino acids that fit into the MHC Class II binding groove. A number of computer algorithms have been developed and used for detecting Class II epitopes within protein molecules of various origins (De Groot A S et al., (1997), AIDS Res Hum Retroviruses, 13(7):539-41; Schafer J R et al., (1998), Vaccine, 16(19):1880-4; De Groot A S et al., (2001), Vaccine, 19(31):4385-95; De Groot A S et al., (2003), Vaccine, 21(27-30):4486-504). These “in silico” predictions of T-helper epitopes have been successfully applied to the design of vaccines and the de-immunization of therapeutic proteins, i.e. antibody-based drugs, Fc fusion proteins, anticoagulants, blood factors, bone morphogenetic proteins, engineered protein scaffolds, enzymes, growth factors, hormones, interferons, interleukins, and thrombolytics (Dimitrov D S, (2012), Methods Mol Biol, 899:1-26). The preferred “therapeutic protein” of the instant invention is human GAA or LYAG.

The EpiMatrix™ system (EpiVax, Providence, Rhode Island) is a set of predictive algorithms encoded into computer programs useful for predicting class I and class II HLA ligands and T cell epitopes. The EpiMatrix™ system uses 20×9 coefficient matrices in order to model the interaction between specific amino acids (20) and binding positions within the HLA molecule (9). In order to identify putative T cell epitopes resident within any given input protein, the EpiMatrix™ System first parses the input protein into a set of overlapping 9-mer frames where each frame overlaps the last by eight amino acids. Each frame is then scored for predicted affinity to one or more common alleles of the human HLA molecule; typically DRB1*0101, DRB1*0301, DRB1*0401, DRB1*0701, DRB1*0801, DRB1*1101, DRB1*1301, and DRB1*1501 (Mack et al., (2013), Tiss Antig, 81(4):194-203). Briefly, for any given 9-mer peptide specific amino acid codes (one for each of 20 naturally occurring amino acids) and relative binding positions (1-9) are used to select coefficients from the predictive matrix. Individual coefficients are derived using a proprietary method similar to, but not identical to, the pocket profile method first developed by Sturniolo (Sturniolo T et al., 1999, Nat Biotechnol, 17(6):555-61, herein incorporated by reference in its entirety). Individual coefficients are then summed to produce a raw score. EpiMatrix™ raw scores are then normalized with respect to a score distribution derived from a very large set of randomly generated peptide sequences. The resulting “Z” scores are normally distributed and directly comparable across alleles.

EpiMatrix™ peptide scoring. It was determined that any peptide scoring above 1.64 on the EpiMatrix™ “Z” scale (approximately the top 5% of any given peptide set) has a significant chance of binding to the MHC molecule for which it was predicted. Peptides scoring above 2.32 on the scale (the top 1%) are extremely likely to bind; most published T cell epitopes fall within this range of scores. Previous studies have also demonstrated that EpiMatrix™ accurately predicts published MHC ligands and T cell epitopes (De Groot A S, Martin W. Reducing risk, improving outcomes: bioengineering less immunogenic protein therapeutics. Clin Immunol. 2009 May; 131(2):189-201.doi: 10.1016/j.clim.2009.01.009. Epub 2009 Mar. 6, herein incorporated by reference in its entirety).

Identification of promiscuous T cell Epitope Clusters. Potential T cell epitopes are not randomly distributed throughout protein sequences but instead tend to “cluster.” T cell epitope “clusters” range from 9 to roughly 30 amino acids in length and, considering their affinity to multiple alleles and across multiple frames, contain anywhere from 4 to 40 binding motifs. Following epitope mapping, the result set produced by the EpiMatrix™ algorithm is screened for the presence of T cell epitope clusters and EpiBars™ by using a proprietary algorithm known as Clustimer™. Briefly, the EpiMatrix™ scores of each 9-mer peptide analyzed are aggregated and checked against a statistically derived threshold value. High scoring 9mers are then extended one amino acid at a time. The scores of the extended sequences are then re-aggregated and compared to a revised threshold value. The process is repeated until the proposed extension no longer improves the overall score of the cluster. Tregitope(s) identified in the present studies were identified by the Clustimer-™ algorithm as T cell epitope clusters. They contain significant numbers of putative T cell epitopes and EpiBars™ indicating a high potential for MHC binding and T cell reactivity.

Identification of tolerogenic T cell Epitope Clusters. The JanusMatrix system (EpiVax, Providence, Rhode Island) useful for screening peptide sequences for cross-conservation with a host proteome. JanusMatrix is an algorithm that predicts the potential for cross-reactivity between peptide clusters and the host genome or proteome, based on conservation of TCR-facing residues in their putative MHC ligands. The JanusMatrix algorithm first considers all the predicted epitopes contained within a given protein sequence and divides each predicted epitope into its constituent agretope and epitope. Each sequence is then screened against a database of host proteins. Peptides with a compatible MHC-facing agretope (i.e., the agretopes of both the input peptide and its host counterparty are predicted to bind the same MHC allele) and exactly the same TCR-facing epitope are returned. The JanusMatrix Homology Score suggests a bias towards immune tolerance. In the case of a therapeutic protein, cross-conservation between autologous human epitopes and epitopes in the therapeutic may increase the likelihood that such a candidate will be tolerated by the human immune system. In the case of a vaccine, cross-conservation between human epitopes and the antigenic epitopes may indicate that such a candidate utilizes immune camouflage, thereby evading the immune response and making for an ineffective vaccine. When the host is, for example, a human, the peptide clusters are screened against human genomes and proteomes, based on conservation of TCR-facing residues in their putative HLA ligands. The peptides are then scored using the JanusMatrix Homology Score. In aspects, peptides with a JanusMatrix Homology Score above 3.0 indicate high tolerogenicity potential and as such may be very useful Tregitopes of the present disclosure

### Example 1. Identification of a Tregitope Compound or Composition

Pompe disease patients lack functional GAA and consequently cannot enzymatically breakdown certain polysaccharides. Recently, it has been identified that enzyme replacement therapy can successfully help these patients and vastly improve their outcomes. However, with the advent of rhGAA/GAA ERT with a recombinant protein, it has been identified that some patients' immune systems will react negatively and recognize the rhGAA as a foreign body. This results in the activation rhGAA specific CD4⁺ T cells and the development of anti-rhGAA antibodies. GAA are is an enzyme critical to breaking down glycogen is the lysosomes of cells to provide simpler saccharides. Mutations in the gene (of which over 200 have been identified) negatively impact functionality and causes glycogen to build up to toxic levels. In some patients, the mutations prevent any enzyme from being produced at all. It is most likely that individuals with a high serum antibody titer recognize epitopes within the rhGAA as foreign. It was therefore identified that ERT could be effective in the patients if the immune system could be trained to dismiss rhGAA as a foreign body.

The complete amino acid sequence of human GAA (Genbank accession: NP_00107327.1) was parsed into overlapping 9-mer frames and scored using the EpiMatrix™ system as described previously.

A purpose built computer program was used to screen putative DRB1*0101, *0301, *0401, *0701, *0801, *0901, *1101, *1301, and *1501 ligands derived from GAA. 18 clusters were identified and screened with the JanusMatrix. FIG. 13. In general, when comparing peptide epitopes in proteins to the human genome, JanusMatrix Human Homology Scores above two are considered significant; indicating an elevated level of conservation between the TCR-facing features of the input peptide or protein and the TCR-facing features of the proteins resident within the human genome. For fully human proteins such as GAA, the threshold can be extended up to three, since as it is known that each TCR motif will be found at least once because the human genome reference database contains a copy of the input sequence. The thresholds used in this analysis to select peptides are: JMX scores 0—<3=Low Conservation; JMX scores ≥3 and <5=Elevated Conservation; and, JMX scores ≥5=High Conservation. Categorization proceeded along the following criteria. A first category grouped epitopes that were predicted to bind across multiple HLA alleles and had a JanusMatrix score ≥2.5 (a typical characteristic of other IgG-derived peptides) identified 11 peptides. (SEQ ID NOS: 2, 5-6, 8, 75, 78, 88-92). A second category considered restricted binding (non promiscuous) epitopes that met the JMX threshold >5 (i.e. meaning that each frame must have at least 5 matches) identified six sequences (SEQ ID NOS: 4, 7, 93-96). This category is of interest where regulatory epitopes could have restricted and specific to the patient's HLA. A third category was used with the same parameters as the second but included peptides with high JMX conservation for DRB1*0401 only. Two sequences were identified within this category, SEQ ID NOS: 97 and 98.

As set forth herein, following HLA binding and TTBSA testing, the sequences were further refined and lead to the identification of six peptides with good activity as Tregitopes. FIG. 1 is the Cluster report and FIG. 2 is the JanusMatrix report for the Tregitope of SEQ ID NO: 2 and the 9-mers contained within SEQ ID NO: 2, including SEQ ID NOS: 9-17. FIG. 3 is the Cluster report and FIG. 4 is the JanusMatrix report for the Tregitope of SEQ ID NO: 1 and the 9-mers contained within SEQ ID NO: 1, including SEQ ID NOS: 9-22. FIG. 5 is the Cluster report and FIG. 6 is the JanusMatrix report for the Tregitope of SEQ ID NO: 3 and the 9-mers contained within SEQ ID NO: 3, including SEQ ID NOS: 9-17-22, 53 and 54. FIG. 7 is the Cluster report and FIG. 8 is the JanusMatrix report for the Tregitope of SEQ ID NO: 5 and the 9-mers contained within SEQ ID NO: 5, including SEQ ID NOS: 30-38. FIG. 9 is the Cluster report and FIG. 10 is the JanusMatrix report for the Tregitope of SEQ ID NO: 6 and the 9-mers contained within SEQ ID NO: 6, including SEQ ID NOS: 39-52. FIG. 11 is the Cluster report and FIG. 12 is the JanusMatrix report for the Tregitope of SEQ ID NO: 4 and the 9-mers contained within SEQ ID NO: 4, including SEQ ID NOS: 23-29. FIG. 12 also depicts several other genes with homology to the sequence set forth in SEQ ID NO: 26. For each of FIGS. 2, 4, 6, 8, 10 and 12 * is the count of HUMAN JanusMatrix matches found in the search database. With respect to a given EpiMatrix Hit (a 9-mer contained within the input sequence which is predicted to bind to a specific allele), a Janus Matrix match is a 9-mer derived from the search database (e.g., the human genome) which is predicted to bind to the same allele as the EpiMatrix Hit and shares TCR facing contacts with the EpiMatrix Hit. Further, the Janus Homology Score** represents the average depth of coverage in the search database for each EpiMatrix hit in the input sequence. For example, an input peptide with eight EpiMatrix hits, all of which have one match in the search database, has a Janus Homology Score of 1. An input peptide with four EpiMatrix Hits, all of which have two matches in the search database, has a Janus Homology Score of 2. The JanusMatrix Homology Score considers all constituent 9-mers in any given peptide, including flanks.

### (2) Methods for the Assessment of Tregitope Binding to Soluble MHC.

Synthesis of peptides. The Tregitopes of the invention can be produced by direct chemical synthesis or by recombinant methods (J Sambrook et al., Molecular Cloning: A Laboratory Manual, (2^{ED}, 1989), Cold Spring Harbor Laboratory Press, Cold Springs Harbor, NY (Publ), herein incorporated by reference in its entirety). Sample Tregitopes were prepared using Fmoc-chemical (9-fluorenylmethoxycarbonyl synthesis, under the guidance and direction of the Inventors of the present invention at 21^{st} Century Biochemicals (Marlborough, Massachusetts). In certain aspects, the Tregitopes were capped with an n-terminal acetyl and c-terminal amino group. HPLC, mass spectrometry and UV scan (ensuring purity, mass and spectrum, respectively) analysis of the selected Tregitopes indicated >80% purity.

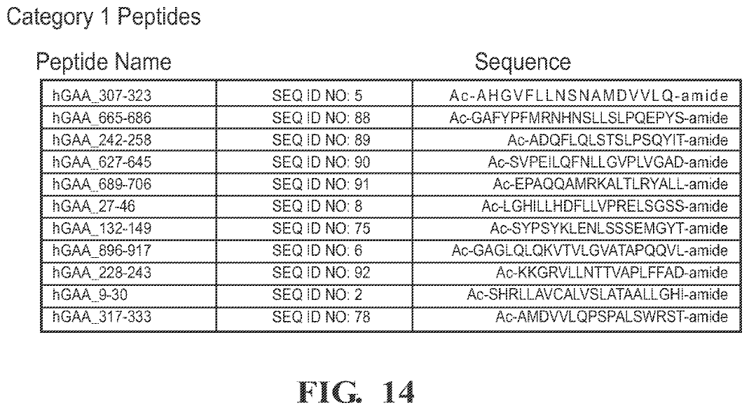

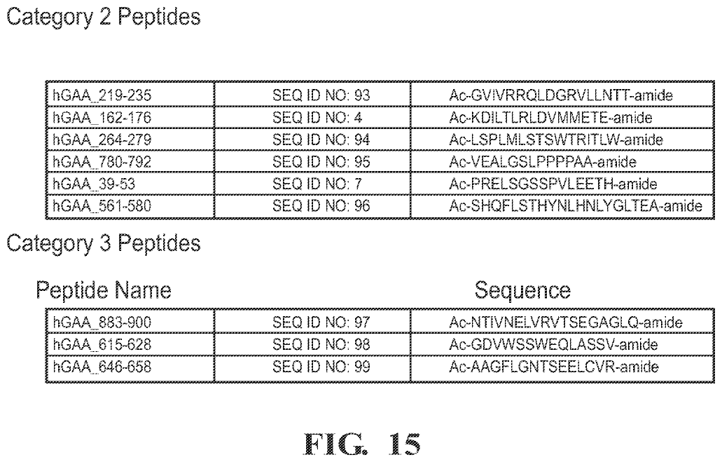

Following the initial Janus Matrix evaluation of the GAA full length enzyme (FIG. 13), eleven category 1 peptides were then synthesized (SEQ ID NOS: 5, 88-92, 75, 78, 2, 6, and 8, six category 2 peptides were also synthesized (SEQ ID NOS: 4, 7, and 93-96), as well as three category 3 peptides (SEQ ID NOS: 97-99) FIGS. 14 and 15.

An amino acid analysis of SEQ ID NOS: 1-6 was conducted by a third-party contractor (New England Peptide, Inc., Gardner, MA) confirming the predicted composition (data not shown). Mass Spectrum and Analytical HPLC analysis was performed by a second independent contractor (21^{St} Century Biochemicals, Inc., Marlboro, MA) further confirming the composition of the Tregitope (data not shown).

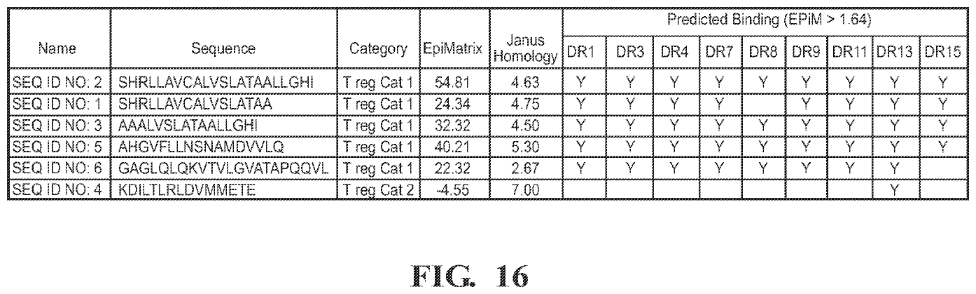

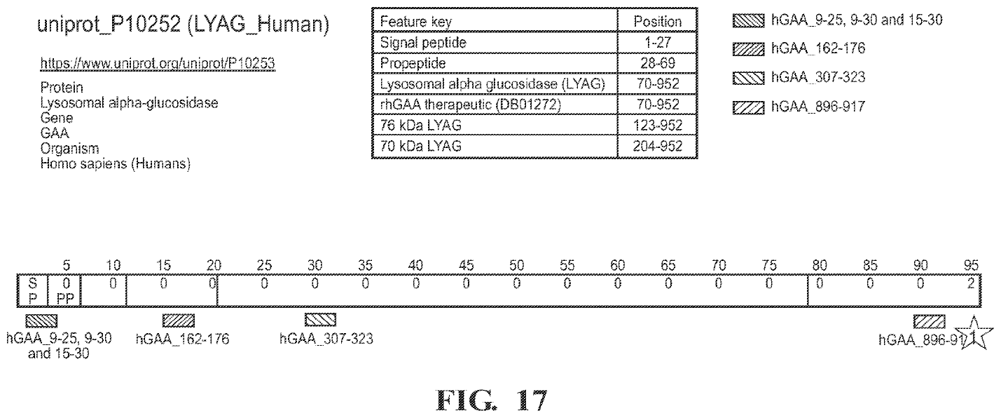

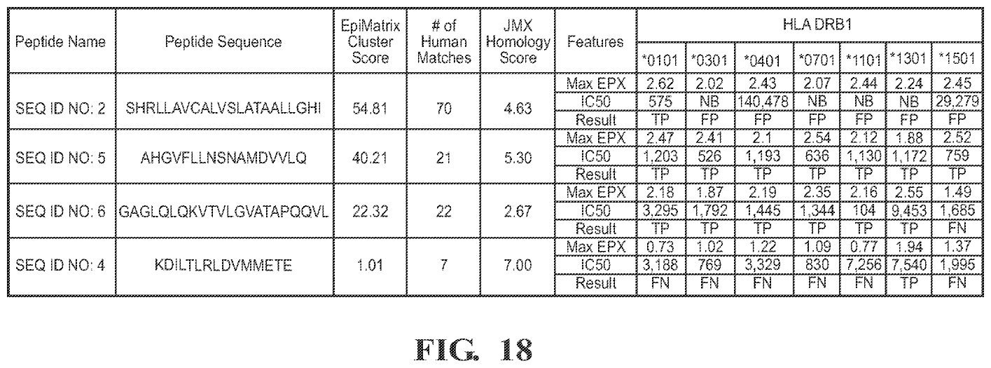

All 20 synthesized peptides were then tested for HLA binding and in a tetanus toxoid bystander suppression assay (TTBSA). The TTBSA identified six peptides for further analysis, including five from category 1 (SEQ ID NOS: 1, 2, 5, 6 and 100) and one from category 2 (SEQ ID NO: 4) (FIG. 23). However, it should be understood that SEQ ID NOS: 7 and 8 also demonstrated Tregitope suppressive activity (data not shown). SEQ ID NOS: 1, 2 and 100 are all associated with the signal peptide of GAA. FIG. 16. The alignment of the peptides with respect to full length GAA is depicted in FIG. 17.

HLA Binding Assay. Binding activity was analyzed at EpiVax (Providence, Rhode Island). The binding assay used (Steere A C et al., (2006), J Exp Med, 2003(4):961-73) yielded an indirect measure of peptide-MHC affinity. Soluble HLA molecules were loaded onto a 96-well plate with the unlabeled experimental Tregitopes and labeled control peptide. Once the binding mixture reached steady equilibrium (at 24 hours), the HLA-Tregitope complexes were captured on an ELISA plate coated with anti-human DR antibody and detected with a Europium-linked probe for the label (PerkinElmer, Waltham, MA). Time-resolved fluorescence measuring bound labeled control peptide is assessed by a SpectraMax® M5 unit (Spectramax, Radnor, PA). Binding of experimental Tregitopes was expressed as the percent inhibition of the labeled control peptide (experimental fluorescence/control fluorescence multiplied by 100). The percent inhibition values for each experimental Tregitope (across a range of molar concentrations) were used to calculate the concentration at which it inhibits 50% of the labeled control Tregitope's specific binding, i.e. the Tregitope's IC₅₀.

Selected experimental Tregitopes were solvated in DMSO. The diluted Tregitopes were then mixed with binding reagents in aqueous buffering solution, yielding a range of final concentrations from 100,000 nM down to 100 nM. Tregitopes were then assayed against a panel of nine common Class II HLA alleles: HLA-DRB1*0101, HLA-DRB1*0301, HLA-DRB1*0401, HLA-DRB1*0701, HLA-DRB1*0801, HLA-DRB1*0901, HLA-DRB1*1101, HLA-DRB1*1301 and HLA-DRB1*1501. From the percent inhibition of labeled control peptide at each concentration, IC₅₀ values were derived for each Tregitope/allele combination using linear regression analysis.

In this assay, the experimental Tregitopes are considered to bind with very high affinity if they inhibit 50% of control peptide binding at a concentration of 100 nM or less, high affinity if they inhibit 50% of control peptide binding at a concentration between 100 nM and 1,000 nM, and moderate affinity if they inhibit 50% of control peptide binding at a concentration between 1,000 nM and 10,000 nM. Low affinity peptides inhibit 50% of control peptide binding at concentrations between 10,000 nM and 100,000 nM. Peptides that fail to inhibit at least 50% of control peptide binding at any concentration below 100,000 nM and do not show a dose response are considered non-binders (NB).

### Example 2. Peptide Characterization by Binding to HLA Class II Molecules

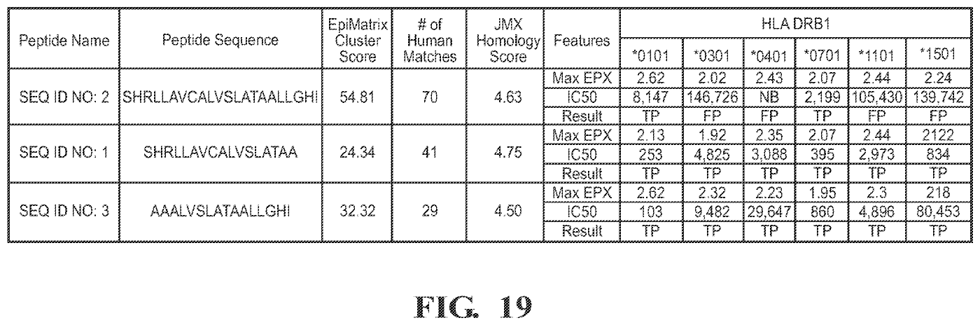

HLA binding for 4 of the peptides was assessed, with SEQ ID NO: 2 also encompassing SEQ ID NO: 1 and SEQ ID NO: 100. These data are presented in FIG. 18 SEQ ID NO: 100 was then redesigned to replace the amino terminal valine and cysteine residues with alanine (SEQ ID NO: 3) and the HLA binding for SEQ ID NOS: 1-3 was re-assessed (FIG. 19).

### (3) Methods for Assessing the Phenotype of Peptide-Exposed APC

Surface expression of Class II HLA (HLA-DR) and CD86 by professional antigen presenting cells (APCs) is one way APCs modulate T cell response. Expression of Class II HLA surface marker is down-regulated in response to Tregitopes, and in particular to, the control Tregitope 167 (21^{st} Century Biochemicals, Marlboro, MA). Additionally, reduced expression of surface marker CD86 correlates positively with enhanced T_{Reg} function (Zheng Y et al., J Immunol, 2004, 172(5):2778-84). In this assay, candidate Tregitopes, including the selected Tregitopes, may be tested for their ability to down-regulate the expression of Class II HLA and the co-stimulatory molecule CD86 on the surface of professional APCs, specifically dendritic cells.

Each of the six Tregitopes of SEQ ID NOS: 1-6 (as well as any Tregitope of the present disclosure, e.g., one or more polypeptides comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) may be individually tested for regulatory potential using a proprietary APC phenotyping assay previously developed at EpiVax (EpiVax, Providence, Rhode Island). Previously harvested and frozen PBMC may be thawed and suspended in chRPMI by conventional means. Under the direction and guidance of the Inventors from EpiVax, HLA typing may be conducted on small, extracted samples of cellular material, provided to EpiVax, by Hartford Hospital (Hartford, Connecticut). On assay day 0, 0.5×10⁶ cells may be extracted, screened for the presence of surface marker CD11c (a marker specific to dendritic cells) and analyzed for the presence of surface markers HLA-DR and CD86 by flow cytometry. The remaining cells may be plated (4.0×10⁶ cell per ml in chRPMI plus 800 ul media) and stimulated (50 μg/mL) with one of the six selected peptides or positive and negative controls including buffer only (negative control), Tregitope 167 (positive control, PAVLQSSGLYSLSLSSVVTVPSSSLGTQ, SEQ ID NO: 111) (21^{st} Century Biochemicals, Marlboro, MA), Flu-HA 306-318 (negative control) (21^{ST} Century Biochemicals, Marlboro, MA) and Ova 323-339 (negative control) (21^{st} Century Biochemicals, Marlboro, MA). Plated cells may be incubated for seven days at 37° C. On assay day 7, incubated cells may be screened by flow cytometry for the presence of surface marker CD11c. CD11c positive cells may be then analyzed for the presence of surface markers HLA-DR and CD86. The experimental peptides may be tested in samples drawn from five different human donors.

Prior to March 2015, all whole blood samples used in the experiments were sourced from healthy donors under IRB 07115 protocol (Clinical Partners, Johnston, RI). Leukocytes were isolated using a conventional ficoll separation gradient (Noble P B and Cutts J H, Can Vet J, 1967, 8(5):110-11). After April 2015, Leukocyte Reduction Filters were obtained from the Rhode Island Blood Center (Providence, RI) to filter the white blood cells from whole blood obtained from healthy donors. After the whole blood was run through the filters, the filters were flushed in the opposite direction to push collected white blood cells out of the filter. The white blood cells were then isolated using a conventional ficoll separation gradient. The collected white blood cells were thereafter frozen for future use. When needed for use in an assay, the frozen white blood cells may be thawed using conventional methods. For the GvHD studies discussed below, PBMCs were obtained from HemaCare, Van Nuys, CA and the experiments were performed at Lifespan Hospital (Providence, RI).

Exposure to putative Tregitopes on the phenotypes of dendritic cells may be measured by multiple means. First, for each experimental condition, dot-plots, contrasting surface expression of CD11c and HLA-DR, may be produced. Dot-plots of cells exposed to all control and experimental peptides may be overlaid onto dot-plots produced from control cells exposed to only the culture media. The overlay may provide an effective method to visually observe shifts in HLA-DR distribution between Tregitope stimulated and unstimulated CD11c-high cells. Observed shifts in the distribution of HLA-DR may be reported as a qualitative measure. Next, the change in intensity of HLA-DR expression for the CD11c-high segment of each dot-plot may be calculated using percent change in intensity of HLA-DR expression being equal to Mean Florescence Index (MFI) of HLA-DR expression for peptide exposed cells minus MFI of HLA-DR expression for media exposed cells divided by MFI of HLA-DR expression for media exposed cells, times 100 (^{HLA-DR}MFI_{peptide}−^{HLA-DR}MFI_{media}/^{HLA-DR}MFI_{media}*100). In this assay, a negative change in observed HLA-DR MFI and a positive change in percentage of HLA-DR-low cells present in the CD11c-high population may indicate reduced expression of HLA and a shift to a regulatory APC phenotype

A similar process may be used to assess the impact of select Tregitope exposure on surface expression of CD86; a costimulatory molecule known to promote T cell activation. First, for each experimental condition, dot-plots contrasting surface expression of CD11c and CD86 may be produced. Dot-plots of cells exposed to all control and experimental Tregitopes may be overlaid onto dots-plots produced from control cells exposed to only the culture media. The overlay provides an effective method to visually observe shifts in CD86 distribution between Tregitope stimulated and un-stimulated CD11c-high cells. Observed shifts in the distribution of CD86 may be reported as a qualitative measure. Next, the change in intensity of CD86-high expression for the CD11c-high segment of each dot-plot may be calculated. Percent change in intensity of CD86-high expression equals Mean Florescence Index (MFI) of CD86 expression for peptide exposed cells minus MFI of CD86-high expression for media exposed cells divided by MFI of CD86 expression for media exposed cells, times 100 (^{CD86-high}MFI_{peptide}−^{CD86-high}MFI_{media}/^{CD86-high} MFI_{media}*100). Next, the percent change in the percentage of CD86-low cells present among the CD11c high population may be calculated. Percent change in the percentage of CD86-high cells equals the percent of CD86-high for peptide exposed cells minus the percent of CD86-high for media exposed cells divided by percent of CD86-high for media exposed cells, times 100 (^{CD86-low}% I_{peptide}−^{CD86-low}%_{media}/^{CD86-low}%_{media}*100). In this assay, a negative change in observed CD86 MFI and a positive change in percentage of CD86-low cells present in the CD11c-high population may indicate reduced expression of CD86 and a shift to a regulatory APC phenotype.

### Example 3. Characterization of Peptide Exposed APC

Dendritic cell phenotyping assays may be performed on the selected Tregitopes according to the methods described previously. Dot-plots corresponding to each experimental condition tested in each of five human donors may then be obtained, such as a series of dot plots representing the surface expression of CD11 vs HLA-DR analyzed on assay day 7 across the donors in the presence of various peptide stimulants. A further series of dot-plots may represent the surface expression of CD11c vs CD86 on assay day 7 across the donors in the presence of various peptide stimulants. Exposure to claimed Tregitopes SEQ ID NOS: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) is expected to decrease expression of CD86 and/or HLA-DR in one or all subjects tested.

### (4) Methods for Assessing Peptide Effects on Proliferation of Regulatory T Cells

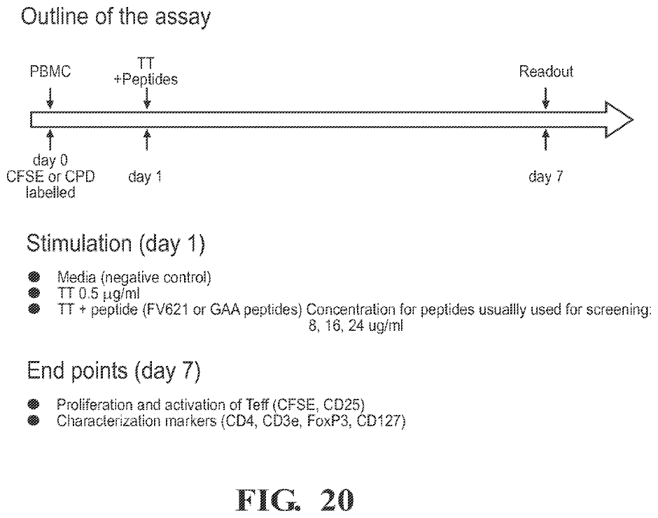

Previous studies performed by EpiVax (Providence, RI) demonstrated increased proliferation of regulatory T cells following exposure to known Tregitope including positive control FV621 (ILTIHFTGHSFIYGK (SEQ ID NO: 74), 21^{st} Century Biochemicals, Marlboro, MA). In this assay, candidate Tregitopes, including the Tregitopes of the instant disclosure, were tested for their ability to induce proliferation among CD4+CD25+FoxP3+ regulatory T cells. Previously harvested and frozen PBMC were thawed and suspended in conditioned chRPMI (3.3×10⁶ cells/mL) by conventional means. Cells were stained with CFSE (Cat #: 65-0850-84, Affymetrix, Santa Clara, CA) and plated at 300,000 cells per well. Plates were incubated overnight (37° C. in 5% CO₂). On assay day 1, SEQ ID NOS: 1-6 and FV621 (control peptide) were reconstituted in sterile DMSO yielding a final stock concentration of 20 mg/mL. Previous titration experiments performed at EpiVax (EpiVax, Providence, Rhode Island) have established that stimulation with 0.5 μg/ml Tetanus Toxoid (TT) (Astarte Biologics, Bothell, WA) elicits a measurable CD4+ effector memory T cells response in PBMC drawn from healthy control donors (Rhode Island Blood Center, Providence, RI). Tetanus Toxoid stock (100 μg/mL) (Astarte Biologics, Bothell, WA) was diluted in conditioned chRPMI yielding a working concentration of 1 ug/mL. Plated cells were then stimulated with either 100 μL of conditioned chRPMI (negative control), 100 μL Tetanus Toxoid solution (positive control) (Astarte Biologics, Bothell, WA), 100 μL of a dilution of 2991 μL Tetanus Toxoid solution plus 8 μg/mL Tregitope solution, 100 μL of a dilution of 2997 μL Tetanus Toxoid solution plus 16 μg/mL Tregitope solution, or 100 μL of a dilution of 6998.2 μL Tetanus Toxoid solution plus 24 μg/mL Tregitope solution. In parallel, control wells with identical number of the same cells were incubated with FV621 peptide solutions prepared similarly. All plates were then incubated for six additional days. On assay day five, 100 μL of supernatant was removed from each well and replaced with freshly conditioned chRPMI. An overview of the assay is depicted in FIG. 20.

### Example 4. SEQ ID NOS: 1-6 Induce a Population of Highly Proliferative, Activated Regulatory T Cells and Inhibit CD4 T Effector Cell Activation and Proliferation

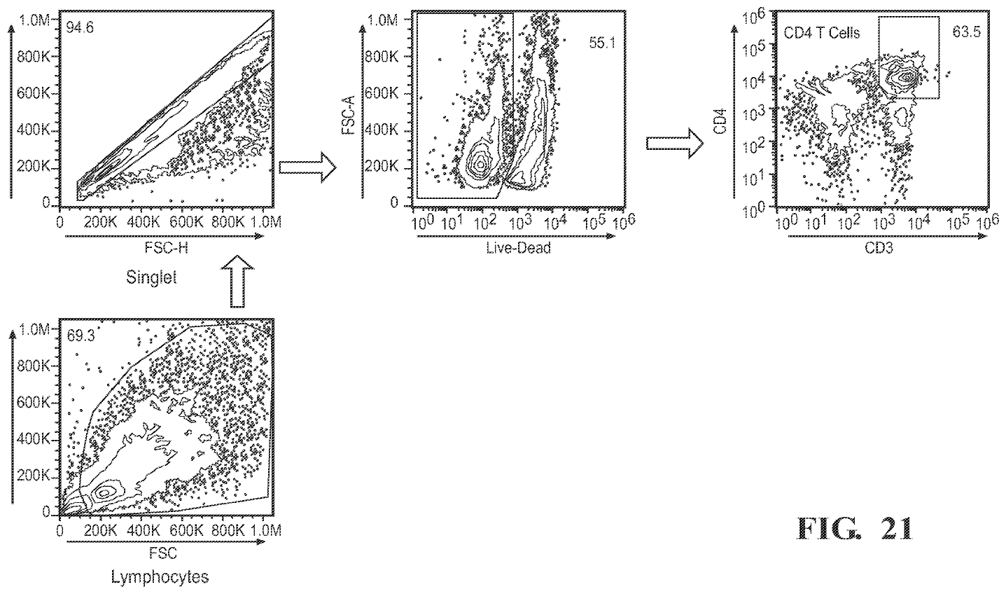

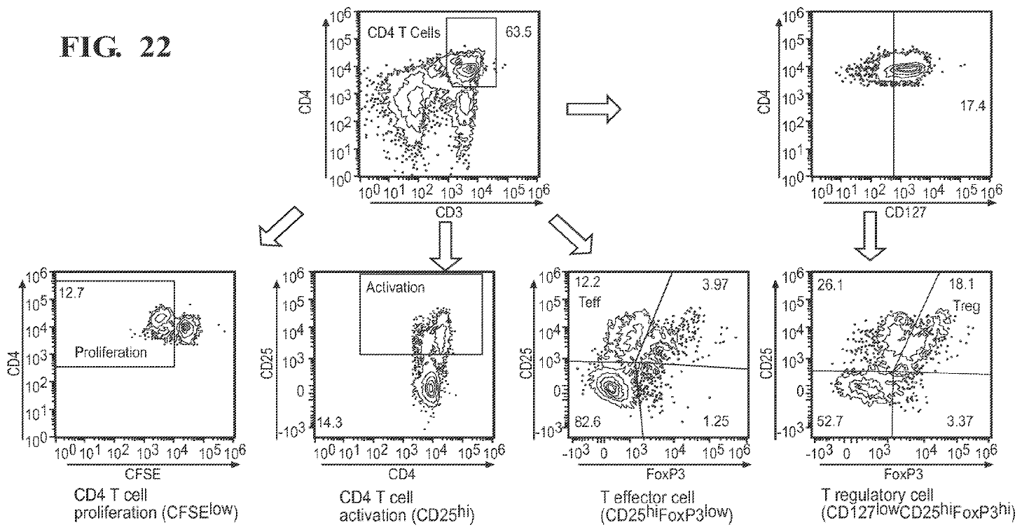

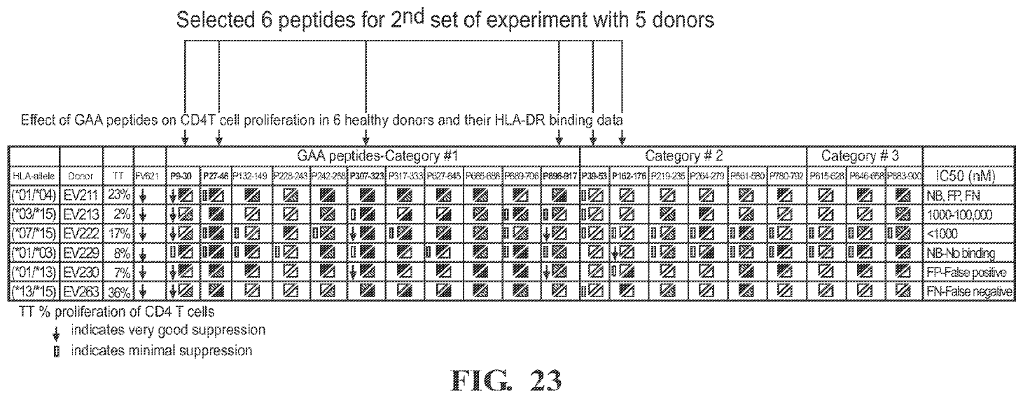

Regulatory T cell proliferation assays were performed on the 20 synthesized Tregitopes of the present disclosure with PBMCs from five donors. We selected highly activated regulatory T cells displaying elevated levels of FoxP3, CD25^{hi}, CFSE^{low}, and CD127^{low}. The gating strategy for highly activated regulatory T cells is shown in FIGS. 21 and 22 (which depict a representative result using Donor 223), in which CD4+ T cells are gated for FoxP3, CD25^{hi}, CD127^{low} and low CFSE (proliferation). FIG. 21 shows the results of the representative assay to obtain CD4+ cells, while FIG. 22 shows the results of the subsequent gating on the CD4+ cells.

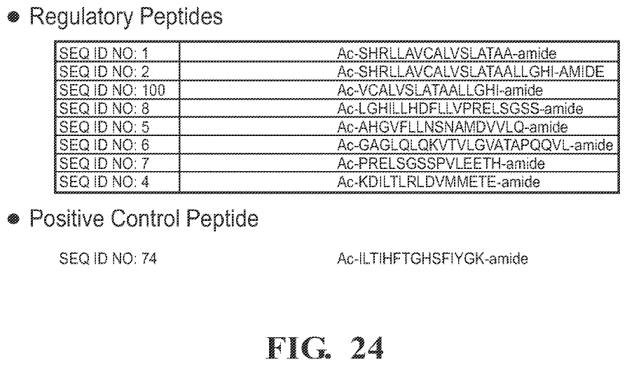

An overview of the results from the 20 synthesized peptides are depicted in FIG. 23. From this, six peptides were selected and an additional two were redesigned for further TTBSA testing. An overview of the 8 peptides and the positive control is presented in FIG. 24. Following further testing, all GAA-regulatory peptides (except SEQ ID NOS: 7 and 8) were shown to inhibit CD4 T cell proliferation (CFSE^{low} expression) activation (CD25^{hi} expression) and T effector cell activation (CD25^{hi}FoxP3^{low}) in a dose dependent manner but the T effector peptides did not have any effect on proliferation. T regulatory cell activation (CD127^{low}CD25^{hi}FoxP3^{hi}) was also observed to be increased in some donors. Further, the ratio of Treg:Teff was also increased by GAA-regulatory peptide stimulation.

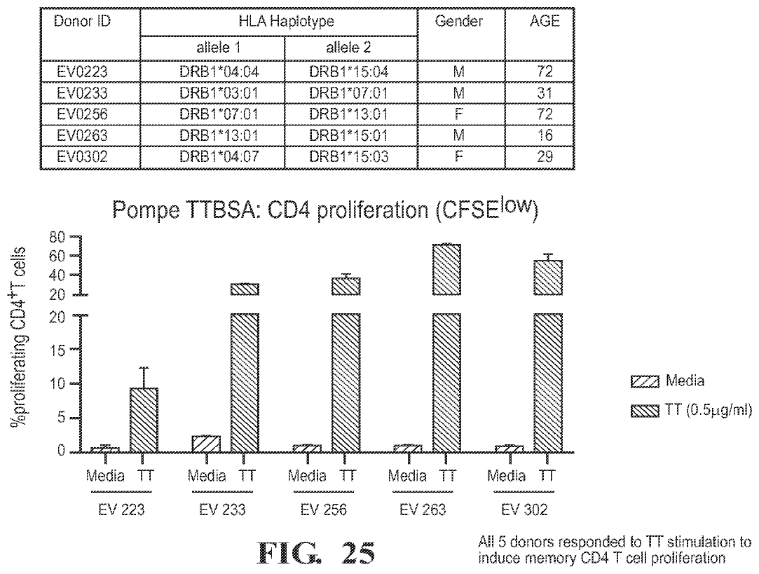

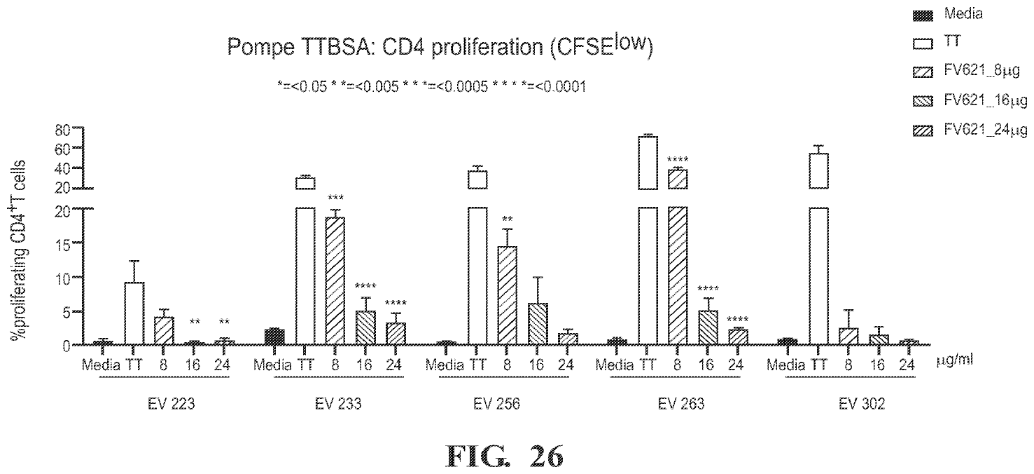

FIG. 25 shows a summary of the donor cells used in the secondary TTBSA testing and that the Tetanus Toxoid caused proliferation in all donor cells. FIG. 26 shows the effect of varying concentrations of the positive control to inhibit the CD4 proliferation.

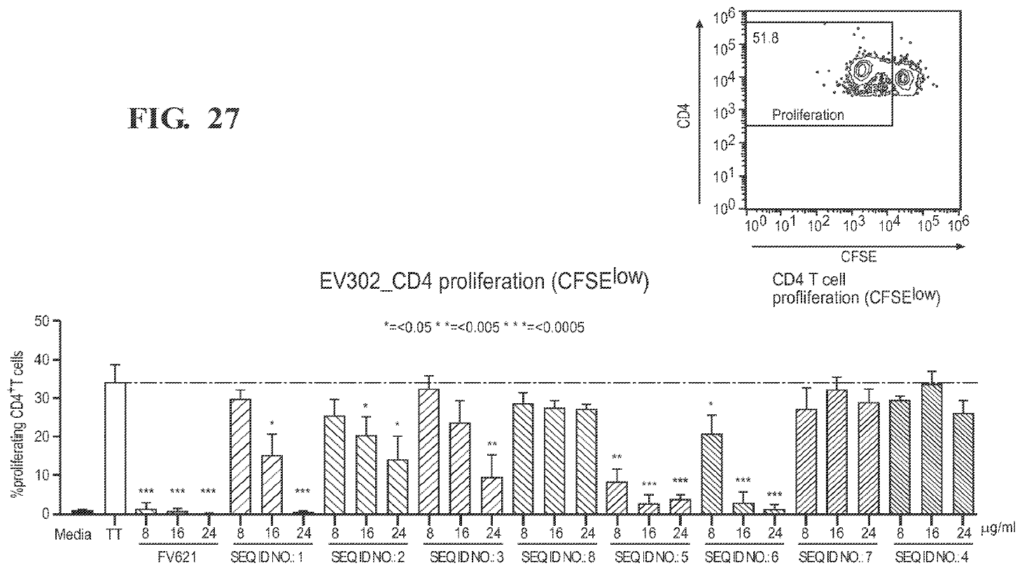

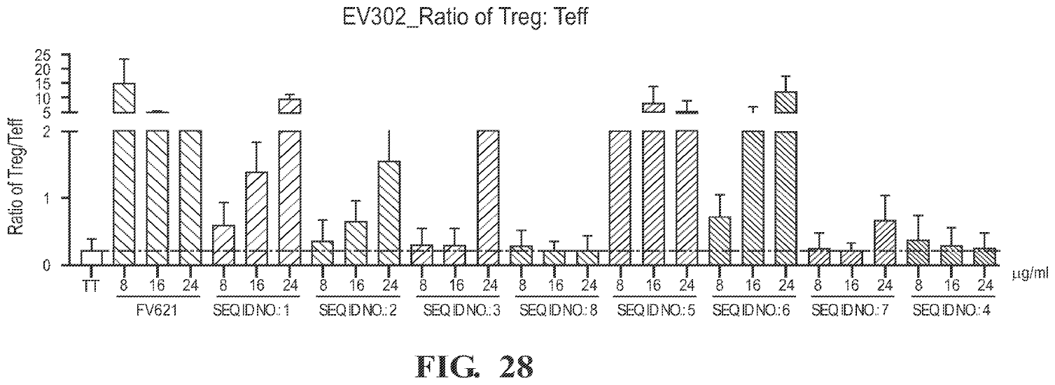

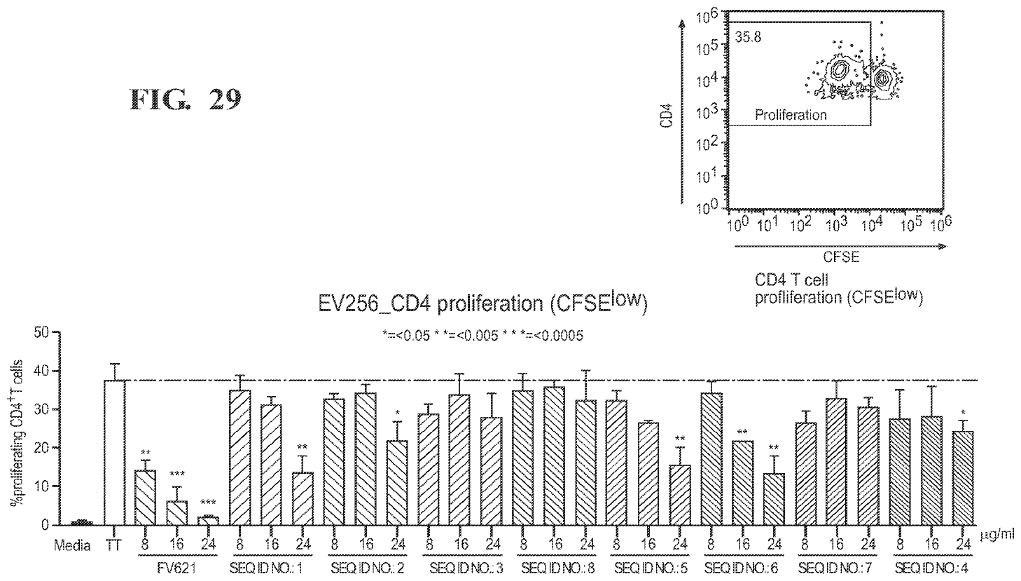

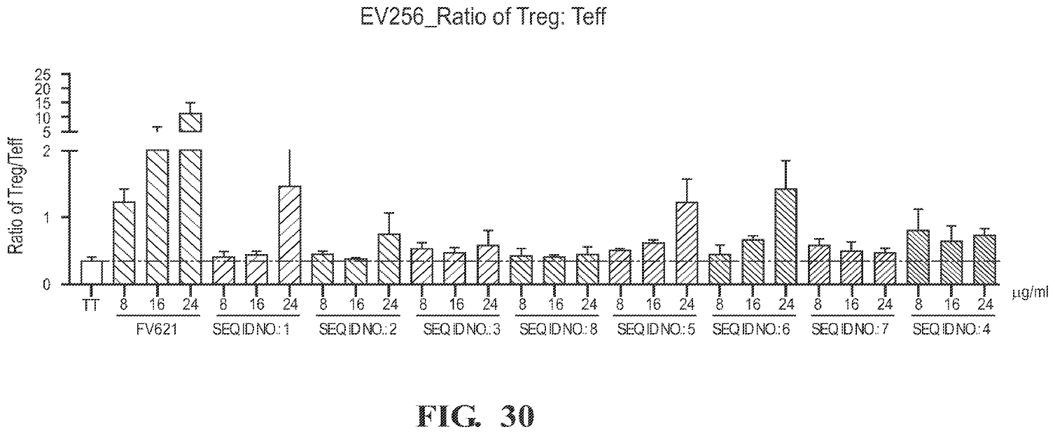

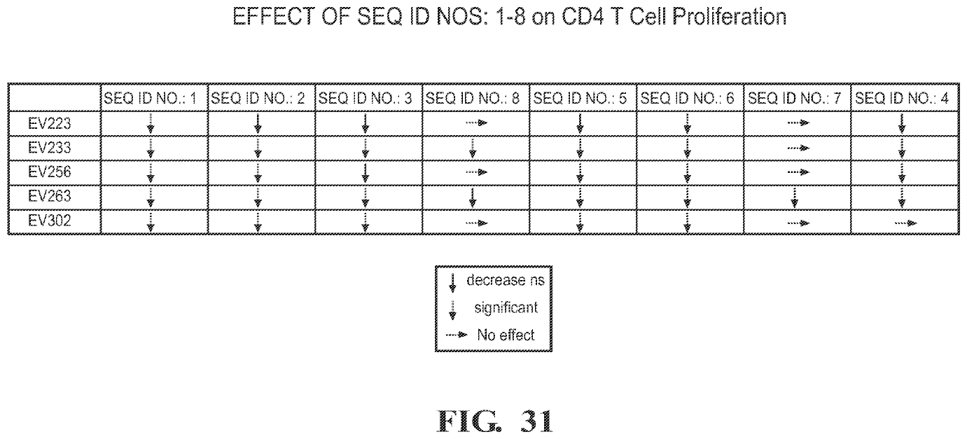

Representative comparisons for the eight tested peptides in two donors are shown in FIGS. 27-30, with FIGS. 27 and 29 showing the effects of the described concentrations of the tested Tregitopes on CD4 proliferation and FIGS. 28 and 30 showing the ratio of Treg to Teff produced in each assay. FIG. 31 provides an overview of the results obtained for all eight peptides. SEQ ID NOS: 1-6 showed good inhibitory activity in some if not all donors cells tested.

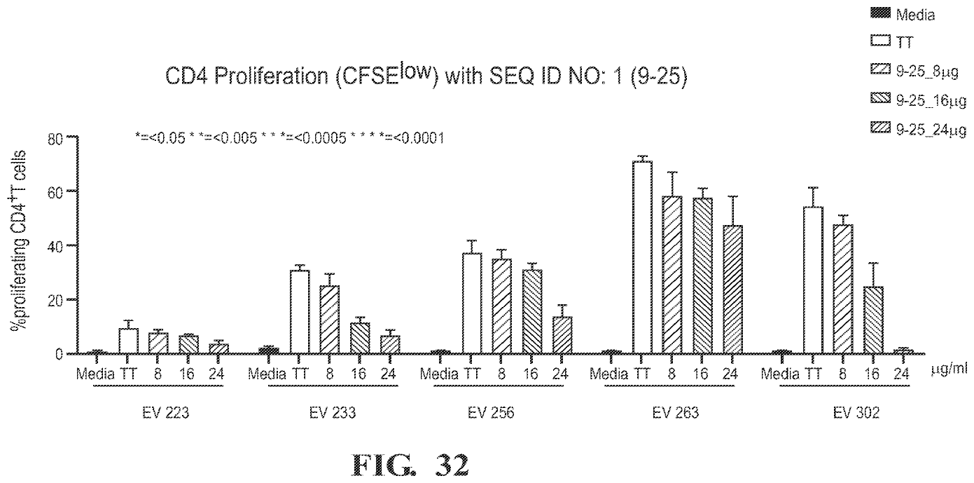

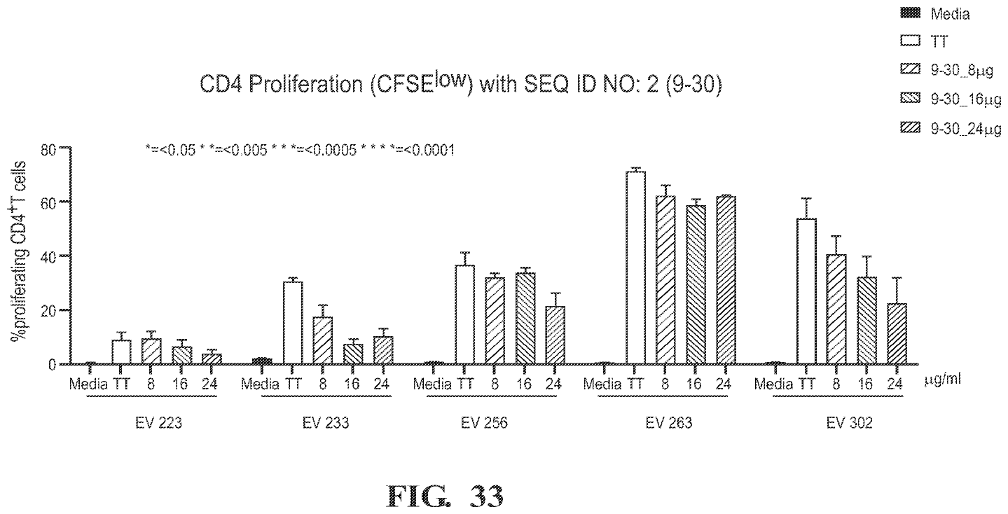

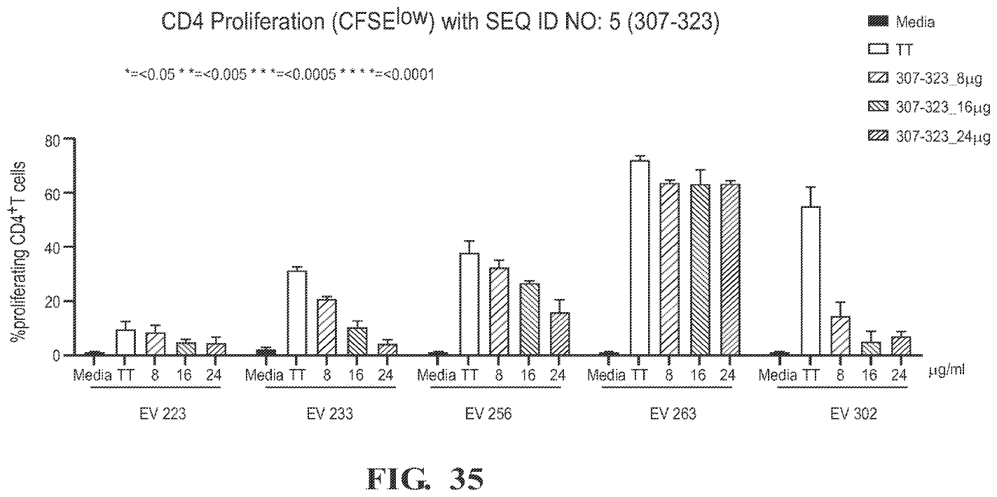

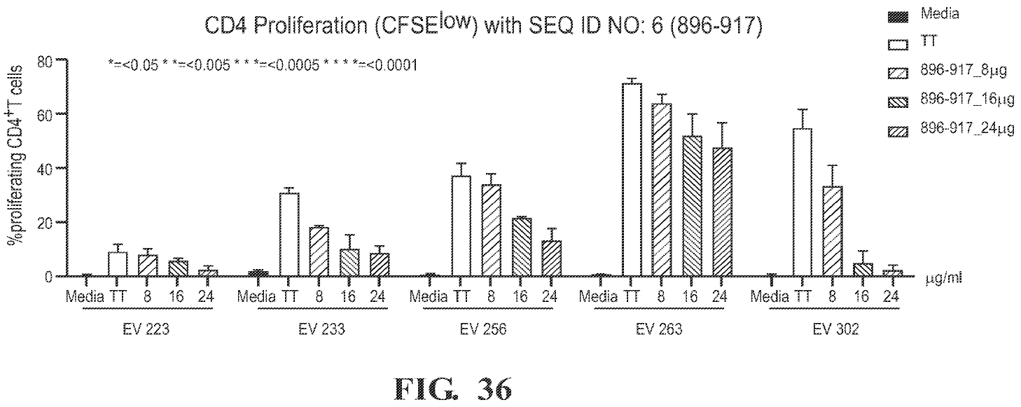

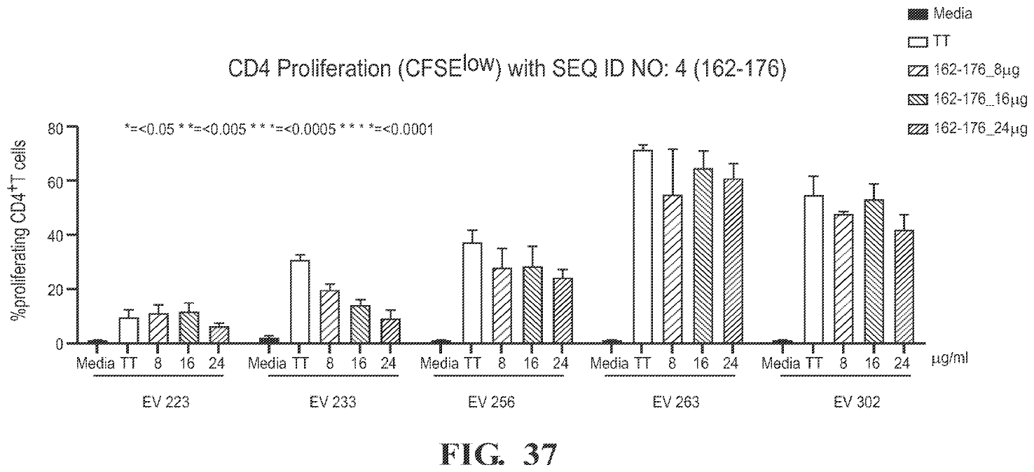

Does response was then evaluated in across all five donors with the identified 6 peptides that showed good inhibition of CD4 T cell proliferation. FIGS. 32-37 show that all six peptides (SEQ ID NOS: 1-6) were able to inhibit CD4 T cell proliferation across all donors. It is expected that a Tregitope of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73) will be able to inhibit CD4 T cell proliferation (CFSE^{low} expression) activation (CD25^{hi} expression) and T effector cell activation (CD25^{hi}FoxP3^{low}), and will be able to increase T regulatory cell activation (CD127^{low}CD25^{hi}FoxP3^{hi}) and/or proliferation in some donors in TTBSA testing.

### (5) Methods for Assessing Peptide Effects on Proliferation of CD4+ Effector T Cells

CD4+ effector memory T cells contained within PBMC cell populations can be induced to proliferate in response to stimulation with known T cell epitopes.

The purpose of this experiment is to establish the ability of a Tregitope of SEQ ID NOS: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) to suppress the proliferation of antigen stimulated CD4+ effector memory T cells by either direct (engagement and activation of T_{Reg}) or indirect (modulation of APC phenotype) means.

Previously harvested and frozen PBMC will be thawed and suspended in conditioned chRPMI (3.3×10⁶ cells/mL) by conventional means. Cells will be stained with CFSE (Cat #: 65-0850-84, Affymetrix, Santa Clara, CA) and plated at 300,000 cells per well. Plates will be incubated overnight (37° C. in 5% CO₂). On assay day 1, a Tregitope of SEQ ID NOS: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) and control peptide will be reconstituted in sterile DMSO yielding a final stock concentration of 20 mg/mL. Previous titration experiments performed at EpiVax (EpiVax, Providence, Rhode Island) have established that stimulation with 0.5 μg/ml Tetanus Toxoid (TT) (Astarte Biologics, Bothell, WA) elicits a measurable CD4+ effector memory T cells response in PBMC drawn from healthy control donors (Rhode Island Blood Center, Providence, RI). Tetanus Toxoid stock (100 μg/mL) (Astarte Biologics, Bothell, WA) will be diluted in conditioned chRPMI yielding a working concentration of 1 ug/mL. Plated cells will then be stimulated with either 100 μL of conditioned chRPMI (negative control), 100 μL Tetanus Toxoid solution (positive control) (Astarte Biologics, Bothell, WA), 100 μL of a dilution of 2991 μL Tetanus Toxoid solution plus 9 μL Tregitope solution, 100 μL of a dilution of 2997 μL Tetanus Toxoid solution plus 3 μL Tregitope solution, or 100 μL of a dilution of 6998.2 μL Tetanus Toxoid solution plus 1.8 μL Tregitope solution. In parallel, control wells with identical number of the same cells were incubated with control peptide solutions prepared as described for Tregitope solution. All plates will then be incubated for six additional days. On assay day five, 100 μL of supernatant will be removed from each well and replaced with freshly conditioned chRPMI.

On assay day seven, cells will be removed from incubation. Cells will be labeled for live/dead discrimination, for surface markers CD127, CCR7, CD4, CD45RA, and CD25 and for intracellular FoxP3. Stained cells are further prepared for FACS analysis by conventional means. Cells are first gated to eliminate aggregates and dead cells. Live cells are gated for CD4 T cells and all subsequent analysis is done on this population. The activated Teffector population is identified as the CD4⁺/CD25-high/FoxP3-intermediate (CD4⁺/CD25^{hi}/FoxP3^{int}) (FIG. 8A). In a parallel analysis of this identified T effector cell population, it can be shown that proliferation of this major population also corresponds to a CD45RA-low and CCR7-low effector memory T cells The major proliferations population corresponds to the T effector memory phenotype (CD45RA-low/CCR7-low).

Proliferation of CD4⁺/CD25-high (CD4⁺/CD25^{hi}) T cells is estimated from the dilution of the CFSE stain (Cat #: 65-0850-84, Affymetrix, Santa Clara, CA) and % proliferation determined by the CFSE-low (CFSE^{lo}) population (FIG. 8B).

### Example 5A. Peptide SEQ ID NO: 1 Suppressed Proliferation and Activation of CD4+ Effector T Cells

The change in activation and proliferation of CD4+ effector cells when the proliferation stimulant (Tetanus Toxoid) is co-delivered with a Tregitope of the instant disclosure, e.g., a Tregitope of SEQ ID NOS: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) is measured and the proliferative response of CD4+ T cells, comprised mainly of T effector memory cells, was characterized.

T cell proliferation assays will be performed on the Tregitopes of the present disclosure according to the methods described previously. Dot plots corresponding to each experimental condition tested for activation and proliferation will be presented. It is expected that a Tregitope of the instant disclosure, e.g. a Tregitope of SEQ ID NOS: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) will strongly suppressed a population of activated effector CD4⁺ T cells (CD4⁺/CD25-high/FoxP3-intermediate, shown as CD4⁺/CD25^{hi}/FoxP3^{int}) reacting to Tetanus Toxoid a dose-dependent manner, while control peptide will have no appreciable effect. Tetanus Toxoid stimulates a population of activated (CD25 high) CD4 T cells to proliferate (CFSE low), with approximately 90% of the activated cells also proliferating (data not shown). This population of highly activated cells is expected to be actively suppressed by Tregitopes of the present disclosure in a dose-dependent manner.

Further, gating on CD4⁺ and CD4− live cells will demonstrate that the inhibitory effect of the Tregitopes of the present disclosure on cell proliferation is more pronounced on the CD4+ population than on the CD4− population.

### (6) Methods for Assessing Peptide Effects on CD8+ Effector T Cells.

CD8+ effector memory T cells contained within PBMC cell populations can be induced to proliferate in response to stimulation with known class I T cell epitopes. SEQ ID NOS: 1-6 may bind multiple HLA alleles and induce a regulatory phenotype in exposed APC (Clinical Partners, Johnston, RI) (the gating strategy employed allows for the identification of the APC fraction in PBMC collected from the whole blood donors). The results of such as assay may establish the ability of SEQ ID NOS: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) to suppress the proliferation of antigen stimulated CD8⁺ T effector memory T cells by either direct (engagement and activation of T_{Reg}) or indirect (modulation of APC phenotype) means.

T cell proliferation assays may be performed on the Tregitopes of the present disclosure according to the methods described previously. PBMCs from two healthy donors may be thawed and suspended in conditioned chRPMI (3.3×10⁶ cells/mL) by conventional means. Cells may be stained with CFSE (Cat #: 65-0850-84, Affymetrix, Santa Clara, CA) and plated at 300,000 cells per well. Plates may then be incubated overnight (37° C. in 5% CO₂). On assay day 1, SEQ ID NOS: 1-6 may be re-constituted in sterile DMSO to yield a final stock concentration of 20 mg/mL. Intermediate solutions of SEQ ID NOS: 1-6 at twice the final concentration in chRPMI may be prepared as described previously. Final concentration of SEQ ID NOS: 1-6 may be tested from 2.5, 5, 10 and 20 ug/ml. As a CD8+ stimulating antigen, the CEF peptide pool which consists of 23 MHC class I restricted viral epitopes derived from human cytomegalovirus, Epstein-Barr virus and influenza virus may be used. CEF peptides may be added to the wells with cells and media (control) or SEQ ID NOS: 1-6 at 0, 1, 2 or 4 ug/ml. All plates may be incubated for six additional days. On assay day 5, 100 uL of supernatant may be removed from each well and replaced with freshly conditioned chRPMI.

Conventional methods may be used to stain cells for live/dead marker, extracellular markers CD4, CD8α and CD25, CD127, CD45RA and CCR7, and intracellular marker FoxP3. After FACS analysis, cells may be gated to eliminate aggregates and dead cells. On the live cells population, CD8α and CD4 cells may be gated separately and each population then analyzed for proliferation (CFSE low population) or activation (CD25-high/FoxP3 low/intermediate, shown as FoxP3int_lo CD25hi) as explained previously.

### Example 6. Suppression of Proliferation of CD8+ Effector T Cells

The potential inhibition of CD8⁺ T cell response by SEQ ID NO: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) when PBMC from healthy donors are stimulated with CEF peptides mixture may be tested. For example, SEQ ID NOS: 1-6 (as well as one or more Tregitopes of the present disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) may strongly inhibits the CD8+ T cell proliferative response to CEF peptides, as well as activation of CD8+ cells. The percentage of proliferating CD8+ T cells (CFSE low) and the percentage of activated CD8+ T effector cells (CD25^{hi}FoxP3^{int/lo}) may be expected to decrease with increasing concentrations of SEQ ID NOS: 1-6, demonstrating that the Tregitopes also have an inhibitory effect on the CD8+ T cell population.

### (7) Methods for Assessing Peptide Effects on Immune Response (GvHD)

Bone marrow transplant is a procedure whereby unhealthy bone marrow is replaced with donated healthy bone marrow. Bone marrow transplants can be used to treat patients with life-threatening blood cancers like leukemia (Vincente D et al., (2007), Bone Marrow Transplant, 40(4):349-54), diseases which result in bone marrow failure like aplastic anemia (Champlin R E et al., (2007), Blood, 109(10):4582-5), and other immune system or genetic diseases (Chinen J and Bucley R H, (2010), J Allergy Clin Immunol, 125(2 Suppl 2):S324-35). Graft versus host disease (GvHD) is known as a major complication in bone marrow transplantation and is characterized by immediate and high mortality after onset (Lee S J et al., (2003), Biol Blood Marrow Transplant, 9(4):215-33). In GvHD, severe tissue damage is caused by donor lymphocytes as they make their way from transplanted donor tissue to HLA-mismatched recipient tissues. Symptoms include severe damage in various organs such as skin, lungs, liver and intestines caused by infusion in the recipient (Goker H et al., (2001), Exp Hematol, 29(3):259-77).

It was previously observed by EpiVax (Providence, RI) that transplantation of human peripheral blood mononuclear cells (PBMCs) (obtained from leukopaks (Hemacare, Van Nuys, CA)) into an immune deficient mouse causes a GvHD-like syndrome resulting in death by 20-50 days. In this model, T cells contained within the transplanted PBMC infiltrate the host mouse's skin, liver, intestine, lungs and kidneys causing severe damage and ultimately death. Immunodeficient mouse strain NOD-scid IL-2Rγ^{null} (NSG—Jax stock #005557) (The Jackson Laboratory, Bar Harbor, ME) mice and transplants of human PBMC may be used to assess the impact of SEQ ID NOS: 1-6 on the progression of GvHD. On assay day-1, mice may be grouped by weight into matched treatment and control groups (Table 9) and then irradiated with 100 cGy from an X-ray irradiator source (Lifespan Hospital, Providence, RI). After 6 hours of irradiation, mice subjects may receive 10 million hPBMCs IV via the tail vein. The mice in Group 8 may receive irradiation, but no PBMCs. Starting on assay day 0 and continuing through assay day 25, subject mice may be dosed according to schedule outlined in Table 9.

Clinical observations, including weight loss, posture, activity, and appearance of hair coat and skin, may be made three times per week. A subject mouse may be euthanized if it exhibits a >20% weight loss from the starting date or exhibits a combination of the following clinical signs: (i) a 10-20% weight loss from the starting date (ii) coldness to touch (iii) lethargy with a hunched posture and scruffy coat.

TABLE 9
Experimental groups and dosing schedule for GVHD study

| | | | | Dose | Dosing |
|---|---|---|---|---|---|
| Group | Mice | PBMC | Test Articles | amount | frequency |
| 1 | 3 | + | PBS (disease | 300 μl | Days 0, 2, 4, 7, |
| | | | control) | PBS | 10, 15, 20, 25 |
| 2 | 10 | + | PBS + DMSO | 0.5% | Days 0, 2, 4, 7, |
| | | | (vehicle) | DMSO | 10, 15, 20, 25 |
| 6 | 10 | + | SEQ ID NO: | 20 μg | Days 0, 2, 4, 7, |
| | | | 1/2/3/4/5/6 | | 10, 15, 20, 25 |
| 7 | 7 | + | IVIG (positive | 50 mg | Days 0, 7, 14, |
| | | | control) | | 21, 28 |
| 8 | 3 | − | None (control | NA | |
| | | | group) | | |

### Example 7. SEQ ID NOS: 1-6 May Inhibit the Development of GvHD in Xenogenic GvHD Model

The transplantation of human lymphocytes into immunodeficient mice and subsequent treatment with one or more Tregitopes of the instant disclosure (e.g., SEQ ID NOS: 1-6 (either alone or in combination), or one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73) may enable the assessment of the instant disclosed Tregitopes on immune function in this in vivo model. For example, SEQ ID NO: 1-6 may suppress T-cell activation thus slowing the progression of the disease. The main evaluation criteria used to evaluate will be survival of the test subjects. A delay in the development of GvHD for the group treated with SEQ ID NO: 1-6 may be observed as suggested by a Kaplan-Meiers Survival Curve.

### Example 8. Generation of a GAA-Tregitope Construct

Fusion of one or more Tregitopes of the instant disclosure (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) with an immunogenic protein can lead to the induction of peripheral tolerance of the immunogenic protein. rhGAA is immunogenic in people receiving ERT to treat Pompe disease. In one exemplary method of producing such constructs, chimeric constructs comprised of the coding sequence of GAA and Tregitope are produced (Sambrook et al., Molecular Cloning: A Laboratory Manual, 2 ed., Cold Spring Harbor Laboratory Press, (1989)). Briefly, the GAA coding region fused at the carboxy-terminus and/or amino-terminus to a Tregitope is generated by annealing overlapping oligos and sub-cloned into an expression plasmid. A Tregitope may also be inserted into GAA SEQ ID NO: 101, e.g. by mutagenesis (i.e., site-directed mutagenesis), particularly if a particular patient has a mutated or missing corresponding section in their own expressed GAA. Similarly, natural variants of SEQ ID NO: 101 can be utilized in the fusion or chimeric proteins or polypeptides of the instant disclosure, including the following variations: S46P, C103G, C103R, C108G, C127F, R190H, Y191C, L208P, P217L, G219RR224P, R224Q, R224W, T234K, T234R, A237V, S251L, S254L, E262K, P266S, P285R, P285S, L291F, L291P, Y292C, G293R, L299R, H308L, H308P, G309R, L312R, N316I, M318K, M318T, P324L, W330G, G335E, G335R, P347R, L355P, P361L, C374R, R375L, G377R, P397L, Q401R, W402R, D404N, L405P, M408V, D419V, 431-433 missing, R437C, R437H, A445P, Y455F, PP457H, P457L, 459 missing, G478R, W481R, P482R, G483V, A486P, D489N, M519T, M519V, E521K, E521Q, P522A, P522S, S523Y, F525Y, S529V, P545L, G549R, L552P, 1557F, C558S, S566P, H568L, N570K, H572Q, Y575C, Y575S, G576R, E579K, R585M, R594H, R594P, S599Y, R6000, R600H, S601L, T602A, 607-612 missing, G607D, A610P, H612Y, H612Y, T614K, G615R, S619R, S627P, N635K, G638V, G638W, L641V, G643R, D645E, D645H, C647W, G648D, G648S, R660H, R672Q, R672T, R672W, 675 missing, R702C, R702L, L705P, R725W, T737N, Q743K, W746G, W746S, Y766C, P768R, R819P, A880D, L901Q, 903 missing, P913R, V916F, N925NGVPVSN, L935P, V949D and combinations thereof. The plasmids are transfected into DG44 CHO cells and stable transfectants selected. The chimeric protein is purified over an immunoaffinity column and evaluated for tolergenicity. Tables 10-12 illustrates exemplary embodiments of such proteins (e.g., a chimeric protein) using SEQ ID NO: 3 with SEQ ID NO: 101. It should be understood in the art that any one of SEQ ID NOS: 1, 2, 4-6 and 9-53 (as well as one or more Tregitopes of the present disclosure as disclosed herein (e.g., one or more polypeptides comprising, consisting, or consisting essentially of an amino acid sequence of SEQ ID NOS. 1-73 (and/or fragments or variants thereof), and optionally 1 to 12 additional amino acids distributed in any ratio on the N terminus and/or C-terminus of the polypeptide of SEQ ID NOS. 1-73)) can be similarly fused or inserted into SEQ ID NO: 101, or the derivatives thereof.

TABLE 10
GAA-Tregitope: SEQ ID NO: 102 (Tregitope bold)

MGVRHPPCSH RLLAVCALVS LATAALLGHI LLHDFLLVPR
ELSGSSPVLE ETHPAHQQGA SRPGPRDAQA HPGRPRAVPT
QCDVPPNSRF DCAPDKAITQ EQCEARGCCY IPAKQGLQGA
QMGQPWCFFP PSYPSYKLEN LSSSEMGYTA TLTRTTPTFF
PKDILTLRLD VMMETENRLH FTIKDPANRR YEVPLETPHV
HSRAPSPLYS VEFSEEPFGV IVRRQLDGRV LLNTTVAPLF
FADQFLQLST SLPSQYITGL AEHLSPLMLS TSWTRITLWN
RDLAPTPGAN LYGSHPFYLA LEDGGSAHGV FLLNSNAMDV
VLQPSPALSW RSTGGILDVY IFLGPEPKSV VQQYLDVVGY
PFMPPYWGLG FHLCRWGYSS TAITRQVVEN MTRAHFPLDV
QWNDLDYMDS RRDFTFNKDG FRDFPAMVQE LHQGGRRYMM
IVDPAISSSG PAGSYRPYDE GLRRGVFITN ETGQPLIGKV
WPGSTAFPDF TNPTALAWWE DMVAEFHDQV PFDGMWIDMN
EPSNFIRGSE DGCPNNELEN PPYVPGVVGG TLQAATICAS
SHQFLSTHYN LHNLYGLTEA IASHRALVKA RGTRPFVISR
STFAGHGRYA GHWTGDVWSS WEQLASSVPE ILQFNLLGVP
LVGADVCGFL GNTSEELCVR WTQLGAFYPF MRNHNSLLSL
PQEPYSFSEP AQQAMRKALT LRYALLPHLY TLFHQAHVAG
ETVARPLFLE FPKDSSTWTV DHQLLWGEAL LITPVLQAGK
AEVTGYFPLG TWYDLQTVPV EALGSLPPPP AAPREPAIHS
EGQWVTLPAP LDTINVHLRA GYIIPLQGPG LTTTESRQQP
MALAVALTKG GEARGELFWD DGESLEVLER GAYTQVIFLA
RNNTIVNELV RVTSEGAGLQ LQKVTVLGVA TAPQQVLSNG
VPVSNFTYSP DTKVLDICVS LLMGEQFLVS WCAAALVSLA
TAALLGHI

TABLE 11
GAA-Tregitope: SEQ ID NO: 103 (Tregitope bold)

AAALVSLATA ALLGHIMGVR HPPCSHRLLA VCALVSLATA
ALLGHILLHD FLLVPRELSG SSPVLEETHP AHQQGA
SRPGPRDAQA HPGRPRAVPT QCDVPPNSRF DCAPDKAITQ
EQCEARGCCY IPAKQGLQGA QMGQPWCFFP PSYPSYKLEN
LSSSEMGYTA TLTRTTPTFF PKDILTLRLD VMMETENRLH
FTIKDPANRR YEVPLETPHV HSRAPSPLYS VEFSEEPFGV
IVRRQLDGRV LLNTTVAPLF FADQFLQLST SLPSQYITGL
AEHLSPLMLS TSWTRITLWN RDLAPTPGAN LYGSHPFYLA
LEDGGSAHGV FLLNSNAMDV VLQPSPALSW RSTGGILDVY
IFLGPEPKSV VQQYLDVVGY PFMPPYWGLG FHLCRWGYSS
TAITRQVVEN MTRAHFPLDV QWNDLDYMDS RRDFTFNKDG
FRDFPAMVQE LHQGGRRYMM IVDPAISSSG PAGSYRPYDE
GLRRGVFITN ETGQPLIGKV WPGSTAFPDF TNPTALAWWE
DMVAEFHDQV PFDGMWIDMN EPSNFIRGSE DGCPNNELEN
PPYVPGVVGG TLQAATICAS SHQFLSTHYN LHNLYGLTEA
IASHRALVKA RGTRPFVISR STFAGHGRYA GHWTGDVWSS
WEQLASSVPE ILQFNLLGVP LVGADVCGFL GNTSEELCVR
WTQLGAFYPF MRNHNSLLSL PQEPYSFSEP AQQAMRKALT
LRYALLPHLY TLFHQAHVAG ETVARPLFLE FPKDSSTWTV
DHQLLWGEAL LITPVLQAGK AEVTGYFPLG TWYDLQTVPV
EALGSLPPPP AAPREPAIHS EGQWVTLPAP LDTINVHLRA
GYIIPLQGPG LTTTESRQQP MALAVALTKG GEARGELFWD
DGESLEVLER GAYTQVIFLA RNNTIVNELV RVTSEGAGLQ
LQKVTVLGVA TAPQQVLSNG VPVSNFTYSP DTKVLDICVS
LLMGEQFLVS WC

TABLE 12
GAA-Tregitope: SEQ ID NO: 104 (Tregitope bold)

MGVRHPPCSH RLLAAAALVS LATAALLGHI LLHDFLLVPR
ELSGSSPVLE ETHPAHQQGA SRPGPRDAQA HPGRPRAVPT
QCDVPPNSRF DCAPDKAITQ EQCEARGCCY IPAKQGLQGA
QMGQPWCFFP PSYPSYKLEN LSSSEMGYTA TLTRTTPTFF
PKDILTLRLD VMMETENRLH FTIKDPANRR YEVPLETPHV
HSRAPSPLYS VEFSEEPFGV IVRRQLDGRV LLNTTVAPLF
FADQFLQLST SLPSQYITGL AEHLSPLMLS TSWTRITLWN
RDLAPTPGAN LYGSHPFYLA LEDGGSAHGV FLLNSNAMDV
VLQPSPALSW RSTGGILDVY IFLGPEPKSV VQQYLDVVGY
PFMPPYWGLG FHLCRWGYSS TAITRQVVEN MTRAHFPLDV
QWNDLDYMDS RRDFTFNKDG FRDFPAMVQE LHQGGRRYMM
IVDPAISSSG PAGSYRPYDE GLRRGVFITN ETGQPLIGKV
WPGSTAFPDF TNPTALAWWE DMVAEFHDQV PFDGMWIDMN
EPSNFIRGSE DGCPNNELEN PPYVPGVVGG TLQAATICAS
SHQFLSTHYN LHNLYGLTEA IASHRALVKA RGTRPFVISR
STFAGHGRYA GHWTGDVWSS WEQLASSVPE ILQFNLLGVP
LVGADVCGFL GNTSEELCVR WTQLGAFYPF MRNHNSLLSL
PQEPYSFSEP AQQAMRKALT LRYALLPHLY TLFHQAHVAG
ETVARPLFLE FPKDSSTWTV DHQLLWGEAL LITPVLQAGK
AEVTGYFPLG TWYDLQTVPV EALGSLPPPP AAPREPAIHS
EGQWVTLPAP LDTINVHLRA GYIIPLQGPG LTTTESRQQP
MALAVALTKG GEARGELFWD DGESLEVLER GAYTQVIFLA
RNNTIVNELV RVTSEGAGLQ LQKVTVLGVA TAPQQVLSNG
VPVSNFTYSP DTKVLDICVS LLMGEQFLVS WC

### Example 9. Generation of a GAA-Multi-Tregitope Construct

Multiple Tregitopes can be present in highly immunogenic proteins to promote adaptive tolerance. In one exemplary method of producing such constructs, chimeric constructs comprised of the coding sequence of GAA and multiple Tregitope(s) are produced (Sambrook et al., Molecular Cloning: A Laboratory Manual, 2 ed., Cold Spring Harbor Laboratory Press, (1989)). Briefly, the GAA coding region fused at the carboxy-terminus and/or to an amino-terminus to Tregitopes is generated by annealing overlapping oligos and sub-cloned into an expression plasmid. Tregitopes may also be inserted into GAA, e.g. by mutagenesis (i.e., site-directed mutagenesis). The plasmids are transfected into DG44 CHO cells and stable transfectants selected. The chimeric protein is purified over an immunoaffinity column and evaluated for tolergenicity. Tables 9 illustrates an exemplary embodiments of such proteins, using carboxy terminal fused SEQ ID NOS: 3 and 5 (e.g., a chimeric protein).

TABLE 11
GAA-Multi-Tregitope: SEQ ID NO: 105(Tregitope
bold)

MGVRHPPCSH RLLAVCALVS LATAALLGHI LLHDFLLVPR
ELSGSSPVLE ETHPAHQQGA SRPGPRDAQA HPGRPRAVPT
QCDVPPNSRF DCAPDKAITQ EQCEARGCCY IPAKQGLQGA
QMGQPWCFFP PSYPSYKLEN LSSSEMGYTA TLTRTTPTFF
PKDILTLRLD VMMETENRLH FTIKDPANRR YEVPLETPHV
HSRAPSPLYS VEFSEEPFGV IVRRQLDGRV LLNTTVAPLF
FADQFLQLST SLPSQYITGL AEHLSPLMLS TSWTRITLWN
RDLAPTPGAN LYGSHPFYLA LEDGGSAHGV FLLNSNAMDV
VLQPSPALSW RSTGGILDVY IFLGPEPKSV VQQYLDVVGY
PFMPPYWGLG FHLCRWGYSS TAITRQVVEN MTRAHFPLDV
QWNDLDYMDS RRDFTFNKDG FRDFPAMVQE LHQGGRRYMM
IVDPAISSSG PAGSYRPYDE GLRRGVFITN ETGQPLIGKV
WPGSTAFPDF TNPTALAWWE DMVAEFHDQV PFDGMWIDMN
EPSNFIRGSE DGCPNNELEN PPYVPGVVGG TLQAATICAS
SHQFLSTHYN LHNLYGLTEA IASHRALVKA RGTRPFVISR
STFAGHGRYA GHWTGDVWSS WEQLASSVPE ILQFNLLGVP
LVGADVCGFL GNTSEELCVR WTQLGAFYPF MRNHNSLLSL
PQEPYSFSEP AQQAMRKALT LRYALLPHLY TLFHQAHVAG
ETVARPLFLE FPKDSSTWTV DHQLLWGEAL LITPVLQAGK
AEVTGYFPLG TWYDLQTVPV EALGSLPPPP AAPREPAIHS
EGQWVTLPAP LDTINVHLRA GYIIPLQGPG LTTTESRQQP
MALAVALTKG GEARGELFWD DGESLEVLER GAYTQVIFLA
RNNTIVNELV RVTSEGAGLQ LQKVTVLGVA TAPQQVLSNG
VPVSNFTYSP DTKVLDICVS LLMGEQFLVS WCAAALVSLA
TAALLGHIAH GVFLLNSNAM DVVLQ

### (10) Generation of Tregitope-Blood Component Conjugates

Fusion of a Tregitope with a blood component conjugate, such as albumin, can be useful as a carrier protein for Tregitope payload. Tregitope-blood component conjugates can extend the half-life of Tregitopes in vivo, protect Tregitopes from rapid proteolytic degradation, protect Tregitopes from rapid clearance from circulation and/or rapid kidney excretion, allow for wide distribution of Tregitope-blood component conjugates throughout the body of a subject, aid in delivery of Tregitopes to appropriate immune cells (such as macrophages and APCs), allow the Tregitopes to be processed by the endocytic pathway of certain immune cells (such as macrophages and APCs), and aid in the presentation of Tregitopes as an antigen by said immune cells.

Tregitope-blood component conjugates may be formed by modifying a Tregitope peptide of the instant disclosure (e.g., but not limited to, a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) by attaching a reactive moiety to the Tregitope peptide to create a modified Tregitope peptide, then forming a bond between reactive moiety of the modified Tregitope peptide with a reactive functionality on a blood component, as disclosed in U.S. Pat. Nos. 6,849,714, 6,887,470, 7,256,253, and 7,307,148. Albumin is a preferred blood component because it contains an Fc neonatal binding domain that will carry the Tregitope-albumin conjugate into the appropriate cells, such as macrophages and APCs. Further, albumin contains a cysteine at amino acid 34 (Cys³⁴) (the location of the amino acid in the amino acid sequence of human serine albumin), containing a free thiol with a pKa of approximately 5, which may serve as a preferred reactive functionality of albumin. Cys³⁴ of albumin is capable of forming a stable thioester bond with maleimidopropionamido (MPA), which is a preferred reactive moiety of a modified Tregitope peptide. The stable thioester bond between albumin and the Tregitope peptide modified with MPA cannot be cleaved under physiological conditions.

The Tregitope peptide may be as disclosed herein, and in certain aspects is preferably selected from SEQ ID NOS: 1-73, or a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73. One or more lysines may be present on the N-terminus of the Tregitope peptide, such as added onto to the N-terminus of peptides selected from SEQ. ID NOS: 1-73. A linker, such as a polyethyleneglycol linker (e.g., PEG2 or PEG12), is present between the one or more lysines and the Tregitope sequence, or at the N-terminus of a Tregitope sequence. In aspects, a lysosomal cleavage site, such as a Cathepsin B site, optionally consisting (sequentially from N-terminus to C-terminus) of valine and citrulline, is present between the PEG2 moiety and the Tregitope sequence. A maleimide-based chemistry may be used to covalently link the modified Tregitope peptide to a blood component, preferably serum albumin, in a 1:1 molar ratio. Linking the modified Tregitope peptide to a blood component may be performed in vivo or ex vivo.

Cathepsin B is the first described member of the family of lysosomal cysteine proteases. Cathepsin B possesses both endopeptidase and exopeptidase activities, in the latter case acting as a peptidyldipeptidase. Cathepsin B was been included in the Tregitope peptide design to facilitate the proper cleavage of the Tregitope from Albumin once it is in the lysosomal compartment in the antigen presenting cells. The Valine-Citrulline is a cathepsin B cleavage site that has been previously used successfully and has been FDA approved in Antibody Drug conjugate (e.g., monomethyl auristatin E (MMAE) conjugate in the drug brentuximab vedotin). Our interest in incorporating the site is to provide cleavage sites that would allow the proper cleavage of the Tregitope from the human serum albumin for efficient MHC class II presentation once it is in the APC. EpiVax sought to determine whether the incorporation of the cathepsin B site is essential to the design of the Tregitope compound or composition.

### Example 10. Generation of a Tregitope-Albumin Conjugate by Ex Vivo Conjugation

Standard Fmoc (9-fluorenylmethoxycarbonyl) solid phase peptide synthesis chemistry can be used for peptide synthesis. Synthesis can be performed on Intavis™ MultiPep™ automated peptide synthesizers. Amino acids can be added stepwise to the growing peptide chain (C-terminus to N-terminus; right to left), while attached to an insoluble polystyrene resin support. Amino acid building blocks, protected at their amino terminus by an Fmoc group, can be coupled to the growing chain after activation of the carboxylic acid terminus via one or more condensation reagents (e.g., Hexafluorophosphate Azabenzotriazole Tetramethyl Uronium (HATU), O—(1H-6-Chlorobenzotriazole-1-yl)-1,1,3,3-tetramethyluronium hexafluorophosphate (HCTU)). The reaction by-products at each addition can be removed by solvent washing (6×, Dimethylformamide (DMF)). Following each coupling and capping step, the Fmoc can be removed via piperidine deprotection of the peptide resin (performed 2×; 20% in DMF volume/volume with 0.1 M HOBt to suppress Asp dehydration), the resin can be washed with DMF 6×, and the next amino acid added. A Cathepsin B cleavage site can be incorporated at the N-terminus of the Tregitope sequence.

For a PEG2 construct (“PEG2” or “P2”), after the desired Tregitope peptide is completed a PEG2 moiety can be added to the N-terminus, followed by the addition of 4 lysines to the N-terminus. The PEG2 and Lysines can be incorporated to provide a potential docking area for the cathepsin B. Additionally, the PEG2 and lysines (via the primary amine on the lysine side-chain) would increase the solubility of the final construct. The composition of the PEG2 construct is shown in Table 10 (below).

TABLE 12
PEG2 construct composition
HSA (Cys 34)-Maleimide linkage-KKKK-Peg2-Val-Cit
(CatB cleavage site) _Tregitope_Nle

For a PEG12 construct (“PEG12” or “P12”), two additions of a PEG6 can be added after the Tregitope peptide synthesis. In this case, no lysines will be added. Increasing the PEG length also provides a docking region for Cathepsin B and improves the solubility of the Tregitope. The composition of the PEG2 construct is shown Table 11 (below).

TABLE 13
PEG12 construct composition
HSA (Cys 34)-Maleimide linkage-Peg12-Val-Cit
(CatB cleavage site) _Tregitope_Nle

Subsequently, a small amount of the peptide constructs can be removed from the resin and the peptide sample cleaved and deprotected by treatment with trifluoroacetic acid (TFA. 92.5% v/v) in the presence of TIS (triisopropylsilane, 5%) and water (2.5%) to scavenge side-chain protecting groups. Each crude, linear, peptide (˜3-5 mg) can be purified by preparative reversed phased HPLC (Gilson) using a 20 mm×50 mm YMC C18, 5 um, Hydrosphere column. The peptides can be purified to >90% purity (determined via analytical HPLC) and the mass verified utilizing an ABI-SCIEX QSTAR XL Pro Qo-TOF mass spectrometer prior to the Cathepsin B evaluation. The remaining peptides (PEG2-Tregitope and PEG12-Tregitope) can be left on the resin for the addition of 3-maleimidopropionic acid (MPA) at a later time.

Recombinant human cathepsin B (catalog 953-CY of R&D Systems™) can be used to evaluate the cleavage of the Val-Cit site engineered into the Tregitope peptide. The activity assay protocol can be used according to the R&D Systems™'s recommendations with final assay conditions of 0.01 ug rhCathepsin B and 10 uM of peptide substrate. After incubation of Cathepsin B with purified peptides (at RT for 15 min). The peptide can be evaluated by mass spec using the Qstar XL Pro™. It can be determined that the PEG2 peptide did not have successful cleavage, and further modification of the Cathepsin B protocol did not produce successful cleavage. For the PEG12 product, successful cleavage can be demonstrated.

After evaluation of the cleavage of the Val-Cit site by Cathepsin B, the reactive moiety of 3-maleimidopropionic acid (MPA) can be added to the N-terminus of the PEG2 and PEG12 peptides. Similar, to the amino acid building blocks, the MPA is protected by an Fmoc group, and coupled to the growing chain after activation of the carboxylic acid terminus. The final MPA-Tregitope constructs can be removed from the resin and the peptide sample can be cleaved and deprotected by treatment with trifluoroacetic acid (TFA. 92.5% v/v) in the presence of TIS (triisopropylsilane, 5%) and water (2.5%). Each crude, linear, peptide (˜20 mg) w can be as purified by preparative reversed phased HPLC (Gilson™) using a 20 mm×50 mm YMC C18, 5 μm, Hydrosphere column. The MPA-peptides can be purified to >90% purity (determined via analytical HPLC) and the mass verified utilizing an ABI-SCIEX QSTAR XL Pro™ Qo-TOF mass spectrometer, as shown in FIGS. 26-29. A total of 15 mg of the MPA-P2 and MPA-P12 Tregitopes can be used in the subsequent conjugation to rHSA (Albucult-Novozyme™) to construct the final preformed HSA-Tregitope conjugate.

Ellman's Reagent (5,5′-dithio-bis-[2-nitrobenzoic acid]) can be used to estimate sulfhydryl groups in a sample by comparing to a standard curve of a sulfhydryl-containing compound such as cysteine. Ellman's test can be performed on rHSA (Sigma™, Albucult®) at multiple concentrations to ensure the accuracy of the analysis. Ellman's reagent (Sigma™), rHSA from Sigma® lot RF-009 can be evaluated for free cysteine that would be available for conjugation with the maleimide. We estimated that 78% of the rHSA had free cysteine available, as shown in Table 12 (below).

TABLE 14
Estimation of free cysteine in rHSA samples

| Grams | | O.D. | | | Moles huHSA per | % free |
|---|---|---|---|---|---|---|
| rHSA | Moles huHSA | 412 | Concentration | Moles | mole free cysteine | cysteine |
| 0.001 | 1.50376E−08 | 0.059 | 4.1696E−06 | 1.16749E−08 | 1.29 | 77.64 |
| 0.002 | 3.00752E−08 | 0.12 | 8.4806E−06 | 2.37456E−08 | 1.27 | 78.95 |

Peptide can be solubilized in dH20, rHSA added (15 mg/ml) and 100 mM Phosphate buffer added to give a final pH of 8. The peptide is added in a 10× molar excess to the HSA. Peptide/HSA can be incubated at room temperature for 2 h followed by incubation at 4° C. for approximately 24-30 hours.

After the conjugation step, the HSA-conjugate can be then dialyzed into PBS (pH 7.0) first at room temperature for 2 hours, followed by 2 changes to fresh PBS at 4° C. for 18-24 h. This process removes excess peptide from the HSA and HSA-Tregitope conjugate preparation.

The Ellman's test can be performed on each conjugate to demonstrate conjugation of the peptide via the rHSA free Cysteine, and determine the efficiency of conjugation in the reaction. The HSA-conjugation preparation does not remove the reduced HSA (mercaptalbumin), inherent in the preexisting preparation (˜22% of the HAS pre-conjugation). The remaining unreacted HSA can be determined to be 14% for the HSA-MPA_P2-Tregitope construct, meaning after conjugation with the maleimide-Tregitope 14% of the free cysteine remained. Thus, ˜64% of total rHSA preparation can be reacted with the MPA_P2-Tregitope peptide.

### (11) Methods for Assessing Effect of Tregitope-Blood Component Conjugates on Immune Cells

A maleimide-based chemistry may be used to covalently link a Tregitope (e.g., but not limited to, a peptide or polypeptide comprising, consisting, or consisting essentially of an amino acid sequence of SEQ ID NOS: 1-73 (and/or fragments or variants thereof), and optionally 1 to 12 additional amino acids distributed in any ratio on the N terminus and/or C-terminus of the polypeptide of SEQ ID NOS: 1-73) payload to recombinant HAS (rHSA) in a 1:1 stoichiometry. Maleimido-propionamido (MPA) forms a stable thiol ester conjugate with the available free Cys34 in HSA. HSA leverages the neonatal receptor (FcRn) recycling pathway, increasing the half-life of any conjugated payload, and potentially decreasing the need for repeat dosing. rHSA is also known to deliver conjugated payloads to the lymph nodes and is endocytosed by dendritic cells and other antigen presenting cells that express FcRn.

EpiVax designed an rHSA-Tregitope conjugate to contain cleavage sites between the Tregitopes. The cleavage sites are specific for an early endosomal protease, which enable the Tregitopes to be liberated from the rHSA molecule, increasing the efficiency of MHC class II presentation on the cell surface. The long and substantiated history of this FDA-Approved rHSA conjugation chemistry approach, as well as its successful manufacturing history support its selection for delivery of our T1 D payload.

Once Tregitope-blood component conjugates are formed, for example as described in Example 14 and subsection 9 of the examples section, as well as the detailed description, the Tregitope-blood component conjugates may be evaluated for their effectiveness in inhibiting effector T-cells and activating regulatory T-cells and their proliferation, for example in comparison with Tregitope peptides alone. Further, the Tregitope-blood component conjugates may be evaluated for their capacity to induce immune tolerance against certain antigens

### Example 11. Evaluation of the Inhibitory Effect of Tregitope-Albumin Delivery Vehicle

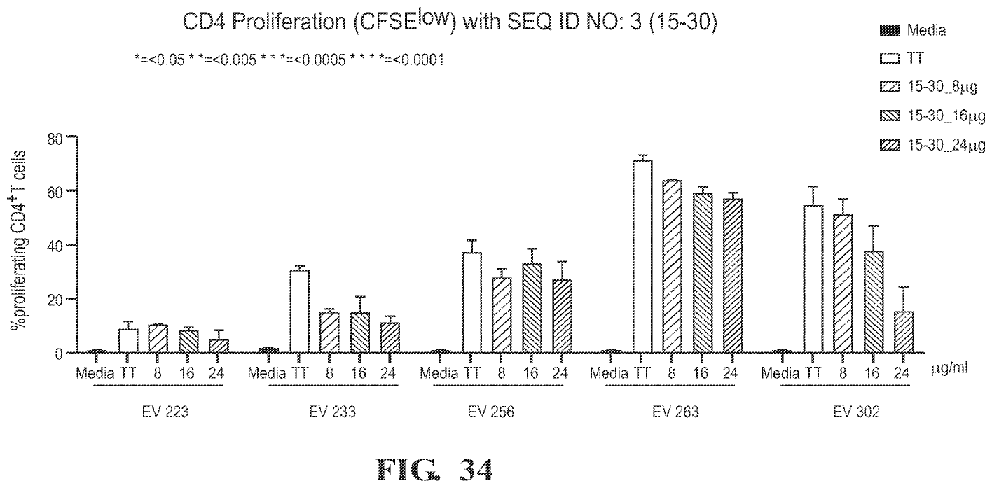

To determine the inhibitory effect of the Tregitope delivery vehicle, healthy donor PBMCs are used in a tetanus toxoid bystander suppression assay (TTBSA), and analysis is done on CD4 T-cell proliferation, activation of T cells, frequencies of T effector and T regulatory cells to determine the ratio of Treg/Teff, as is displayed in FIG. 34.

So as to optimize the best combination of Tregitopes for translation to the clinic, the effect of combinations of Tregitopes for their ability to synergistically suppress effector T-cell responses in vitro is analyzed. To facilitate these comparisons, a high throughput in vitro assay is developed using human donor peripheral blood mononuclear cells (PBMCs). This assay, referred to as the Tetanus Toxoid Bystander Suppression Assay, takes advantage of the ability of Tregs to suppress T memory cells specific to Tetanus that are elicited in individuals with a history of Tetanus toxoid (TT) vaccination.

At day 0, PBMCs are incubated and stained with Carboxyfluorescein succinimidyl ester (CFSE) dye. At day 1, cells are stimulated with by adding media, Tetanus Toxoid, and either: 8, 6, or 24 μg/mL of a Tregtiope; or 10, 40, or 100 μg/mL of a Tregtiope-albumin conjugate. Tetanus Toxoid is used at a final concentration of 0.5 μg/ml, where the concentration is methodically titrated and optimized to measure the inhibitory capacity of Tregitopes. Negative controls, including media-only, are included. At day 7, L/D cell population marker, extracellular stain, and intracellular stain are added to the cells. At day 8, a readout is taken. Cell sorting assays for analysis of activation markets (e.g., CFSE, CD25) and cell population markets (e.g., L/D, CD2c, CD4, and FoxP3) are performed.

Incubation of donor PBMCs with TT stimulates expansion of T effector cells. Tregitopes are added to PBMC in vitro with TT, and activate CD25^{hi}FoxP3^{hi} regulatory T cells suppressing expansion of TT-specific T effector cells. Tregitopes significantly inhibit the proliferation (as is measured by CFSE dilution) and activation (as is measured by CD25 expression) of CD4+ T effector cells in a dose dependent manner, and also slightly expand Tregs (CD25⁺/FoxP3⁺/CD127^{lo}), which is suggested by an increase in the ratio of Treg/T_{eff} cells. A reduction of effector T cell proliferation is a direct consequence of the activation of T regulatory cells and/or the conversion of TT-specific T effector to Treg, for example as is supported by the induction of Treg in vivo.

Using the TTBSA, each of a number of available Tregitopes individually and in pairwise combinations is examined for their potential to suppress CD4+ T cell proliferation. The most promising IgG-Tregitope peptides are selected for further testing. A certain Tregitope, Tregitope A, is the single Tregitope has the most suppressive activity in the TTBSA as compared to the other single Tregitopes. Combining Tregitope A with Tregitope C, an even greater suppressive effect on TT-specific T cell proliferation is observed. Conjugating A+C to rHSA improves their efficacy in vitro.

Thus, using TTBSA, it is shown that HSA-Tregitope conjugates inhibit CD 4 T-cell proliferation and activation, and increase the ratio of Treg cells to T_{eff} cells.

### Example 12. Evaluation of the Effectiveness of Preformed Conjugate HSA-Tregitope Therapeutics and Maleimide-Tregitope Peptide Therapeutics

The effect on the response to OVA immunization of preformed conjugate HSA-Tregitope therapeutics and a free-maleimide-Tregitope peptide is evaluated. The latter free-maleimide peptide forms a conjugation in vivo after injection via the reactive maleimide group to the free-Cys34 of the subject's endogenous HSA. 5 mgs of the MPA-P2 and MPA-P12 is used as free-MPA-Tregitope, with the unconjugated HSA in the sample being accounted for by calculating the molar ratio of conjugated to unconjugated HSA.

Mice (female C57BL/6) are immunized s.c. with 50 mg ovalbumin (OVA) on day 0 (CFA) and day 14 (IFA). The preformed HSA conjugate treatments is administered with the OVA in CFA on day 0. Test groups include OVA/HSA-P2-high and OCA/HSA-P2-low. Per injection OVA is 50 g, and HSA at 800 μg, and HSA-P2H(high) conjugation is at 825 μg (˜20 μg Tregitope). HSA-P2L(low) conjugation is at 100 μg (˜3.7 μg Tregitope). Four control groups include PBS only, PBS/OVA, HSA/OVA, and Tregitope/OVA. A last arm is included to evaluate the utility of the free-maleimide Tregitope peptide and is administered by IV into tail vein. There are five mice per group.

Mice are sacrificed on Day 17. Upon sacrifice, cardiac bleeds and spleens are harvested for each animal. IFNγ/IL2 fluorospot assays, IFNγ/IL17 fluorospot assays, CD4 T cell proliferation, and T cell characterization are performed on the splenocytes stimulated with OVA. PHA is used as a positive control stimulation for spleen cell assays. All of the wells in PHA stimulation are confluent. An acceptance criteria is used wherein SFC (spot forming cells) after stimulation must be greater than 50 spots/106 over negative control (media wells) and must also have a stimulation index greater than 2. According to both the IFNγ/IL2 fluorospot and IFNγ/IL17 fluorospot assays, IFNγ production is inhibited by treatment, and the HSA-only control group is inhibited less compared than the treatment groups.

For T-cell proliferation and characterization assays, splenocyte samples are evaluated for induction of FoxP3 expression in TCR Tg cells and for the suppression of OVA specific T cell proliferation (in response to OVA peptide in vitro) by CFSE dilution. To detect FoxP3⁺ Tregs, a single-cell suspension of draining lymph nodes is incubated with 2.4G2 mAb (anti-CD16/32, ATCC) for 15 minutes to block FcR then is stained with anti CD3, CD4, CD25 and anti-clonotypic KJ1-26 for 40 minutes at 4° C. KJ1-26 is specific for clonotypic TCR expressed by DO11.10 transgenic mice. Cells are then be permeabilized and stained for FoxP3 nuclear expression and acquired on a Thermo Attune N×T Autosampler™ for FACS analysis. The CD4⁺CD25⁺FoxP3⁺KJ1-26⁺ live cell gate population is established to determine the number and proportion of OVA-Specific T regulatory cells compared to PBS or HSA alone.

Antigen-specific T cell proliferation is evaluated by CFSE dilution. Draining lymph nodes are harvested, are stained with cell proliferation dye CFSE, and a single-cell suspension is prepared at 2×10⁶ cells/mL. Cells are added to 96-well plates at 100 μL per well in the presence of 10 μg/ml concentration of OVA 323-339 (New England Peptide, Gardner, MA, USA). Cells are stimulated for 72 hours and harvested for staining with CD3a, CD4, CD8, CD54RA, CCR7, CD25, CD127, IFNγ HLA-II, CD69, CD154, IL-17, IL-21 for 40 minutes at 4° C. Cells are be fixed, permeabilized and stained for FoxP3 expression and analyzed by flow cytometry. An increase of OVA-specific KJ1-26⁺CD4⁺CD25⁺FoxP3⁺ adaptive (converted) T regulatory cells in mice treated with free maleimide-Tregitopes and HSA-Tregitope conjugates as compared to mice treated with rHSA is observed. free maleimide-Tregitopes and HSA-Tregitope conjugates more effectively reduces OVA-specific proliferation of KJ1-26⁺CD4⁺ T effector cells as compared to rHSA alone.

Anti-OVA antibodies in serum from the bleeds harvested on day 17 are evaluated in serum by ELISA, including a serial dilution plot and a standard ELISA to determine antibody concentrations. Mice treated with HSA-conjugates and free maleimide have lower serum antibody titers compared to no treatment, as indicated by absorbance at different dilutions, as well as comparison of absorbance over a standard curve.

### EQUIVALENTS

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains, and as fall within the scope of the appended claims.

1. A polypeptide consisting of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

2. A polypeptide consisting essentially of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

3. A polypeptide comprising an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

4. A nucleic acid encoding a polypeptide consisting of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

5. A nucleic acid encoding a polypeptide consisting essentially of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

6. A nucleic acid encoding a polypeptide comprising an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

7. A vector comprising the nucleic acid according to any one of claims 4-6.

8. A cell comprising the vector according to claim 7.

9. A method for suppressing an immune response in a subject in need thereof, the method comprising administering to the subject a therapeutically effective amount of one or more isolated regulatory T-cell epitopes selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

10. The method according to claim 9, wherein the immune response is a result of treatment with at least one or more therapeutic treatments with at least one therapeutic protein, treatment with a vaccine or treatment with at least one antigen.

11. The method according to claim 9, wherein said regulatory T-cell epitope is administered to isolated dendritic cells ex vivo, and said dendritic cells are the re-introduced to the subject.

12. The method according to claim 9, wherein the administration of the regulatory T-cell epitope shifts one or more antigen presenting cells to a regulatory phenotype.

13. The method according to claim 9, wherein the administration of the regulatory T-cell epitope shifts one or more dendritic cells to a regulatory phenotype.

14. The method according to claim 13, wherein the regulatory phenotype is characterized by a decrease in CD11c and HLA-DR expression in the dendritic cells or other antigen presenting cells.

15. The method according to claim 9, wherein the administration of the regulatory T-cell epitope shifts one or more T cells to a regulatory phenotype.

16. The method according to claim 15, wherein the administration of the regulatory T-cell epitope shifts one or more CD4⁺ T cells to a regulatory phenotype.

17. The method according to claim 15, wherein the administration of the regulatory T-cell epitope shifts one or more CD8⁺ T cells to a regulatory phenotype.

18. The method according to claim 15, wherein the administration of the regulatory T-cell epitope shifts one or more B cells to a regulatory phenotype.

19. The method according to claim 9, wherein the administration of the one or more regulatory T-cell epitopes activates CD4⁺/CD25⁺/FoxP3+ regulatory T-cells.

20. The method according to claim 9, wherein the administration of the one or more regulatory T-cell epitopes suppresses activation of CD4⁺ T-cells.

21. The method according to claim 9, wherein the administration of the one or more regulatory T-cell epitopes suppresses activation or proliferation of CD4⁺ and/or CD8⁺ T-cells.

22. The method according to claim 9, wherein the administration of the one or more regulatory T-cell epitopes suppresses activation or proliferation of B-cells.

23. The method according to claim 9, wherein the administration of the one or more regulatory T-cell epitopes suppresses an immune response selected from the group consisting of an innate immune response, an adaptive immune response, an effector T cell response, a memory T cell response, a helper T cell response, a B cell response, a ηκT cell response, or any combination thereof.

24. A composition comprising an effective amount of one or more isolated regulatory T cell epitopes and/or fragments and variants thereof according to claim any one of claims 1-3 and one or more immune stimulating T-cell epitope polypeptides, wherein said composition suppresses the immune response activated by said immune stimulating T-cell epitope polypeptide.

25. The composition according to claim 24, wherein said one or more immune stimulating T-cell epitope polypeptides is one or more therapeutic protein, treatment with a vaccine or treatment with at least one antigen.

26. A pharmaceutical composition comprising one or more isolated regulatory T cell epitope (Tregitope) effective to suppress an immune response in a human, wherein at least one isolated regulatory T cell epitope comprises an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

27. The pharmaceutical composition according to claim 26, wherein the at least one regulatory T cell epitope peptide consists of an amino acid sequence selected from the group consisting of SEQ ID NOS: 1-6.

28. A pharmaceutical composition comprising the Tregitope compound or composition according to claim 26 and a pharmaceutically acceptable carrier.

29. A method for stimulating regulatory T-cells in a subject in need thereof, the method comprising administering to the subject a therapeutically effective amount of a composition of claim 26.

30. A method for suppressing an immune response in a subject in need thereof, the method comprising administering to the subject a therapeutically effective amount of a composition of claim 26.

31. A method for suppressing an antigen-specific immune response in a subject in need thereof, the method comprising administering to the subject a therapeutically effective amount of a composition of claim 26.

32. The method according to claim 31, wherein the subject suffers from an allergy, an autoimmune disease, Pompe disease, a transplant related disorder, an enzyme or protein deficiency disorder, a hemostatic disorder, cancer, infertility; and a viral, bacterial or parasitic infection or a blood clotting disorder.

33. The method according to claim 31, wherein the immune response is a result of one or more therapeutic treatments selected from the group consisting of treatment with at least one therapeutic protein, treatment with a vaccine, and treatment with at least one antigen.

34. The method according to claim 31, wherein the administration of the pharmaceutical Tregitope compound or composition shifts one or more antigen presenting cells to a regulatory phenotype.

35. The method according to claim 31, wherein the administration of the pharmaceutical Tregitope compound or composition shifts one or more dendritic cells to a regulatory phenotype.

36. The method according to claim 35, wherein the regulatory phenotype is characterized by a decrease in CD11c and HLA-DR expression in the dendritic cells or other antigen presenting cells.

37. A method for suppressing an immune response in a subject in need thereof, the method comprising administering to the subject a therapeutically effective amount of a Tregitope compound or composition comprising one or more isolated polypeptides, wherein at least one isolated polypeptide consists of an consists of an amino acid sequence selected from the group consisting of SEQ ID NOS: 1-6.

38. The method according to claim 37, wherein the administration of the Tregitope compound or composition activates CD4⁺/CD25⁺/FoxP3⁺ regulatory T-cells.

39. The method according to claim 38, wherein the administration of the Tregitope compound or composition suppresses activation of CD4⁺ T-cells.

40. The method according to claim 38, wherein the administration of the Tregitope compound or composition suppresses activation or proliferation of CD4⁺ effector T-cells and/or CD8⁺ effector T-cells.

41. The method according to claim 38, wherein the administration of the Tregitope compound or composition suppresses activation or proliferation of B-cells.

42. The method according to claim 38, wherein the subject suffers from an allergy, an autoimmune disease, a transplant related disorder, an enzyme or protein deficiency disorder, or a blood clotting disorder.

43. The method according to claim 38, wherein the immune response is a result of one or more therapeutic treatments select from the group consisting of, treatment with at least one therapeutic protein, treatment with a vaccine, and treatment with at least one antigen.

44. A kit for suppressing an immune response in a subject, wherein the kit comprises a composition according to claim 26.

45. The kit according to claim 44, further comprising an effective amount of an antigen or allergen.

46. A method for expanding a population of regulatory T cells of a patient, comprising:
(a) providing a biological sample obtained from a subject; and
(b) isolating regulatory T-cells from the biological sample; and contacting the isolated regulatory T-cells with an effective amount of a Tregitope compound or composition of claim 26 under conditions wherein the T-regulatory cells increase in number to yield an expanded regulatory T-cell composition, thereby expanding the regulatory T-cells in the biological sample; and
(c) returning said increased number of regulatory T cells to said patient.

47. A method for stimulating regulatory T cells in a biological sample, comprising:
(a) providing a biological sample obtained from a subject;
(b) isolating regulatory T-cells from the biological sample; and contacting the isolated regulatory T-cells with an effective amount of a composition of claim 26 under conditions wherein the T-regulatory cells are stimulated to alter one or more biological function, thereby stimulating the regulatory T-cells in the biological sample.

48. A composition according to claim 26, wherein the regulatory T cell epitope is either covalently bound, non-covalently bound or in admixture with a specific target antigen for use in the diminution of immune response against the target antigen.

49. The composition according to claim 48, wherein the suppressive effect is mediated by a natural T_{Reg} or an adaptive T_{Reg} or a viral homolog of the natural T_{Reg}.

50. The composition according to claim 49, wherein any of effector T cells, helper T cells, or B cells are subject to the suppressive effect of the regulatory T cell epitope.

51. A polypeptide composition comprising one or more T-cell epitope polypeptides linked to a heterologous polypeptide, wherein the T-cell epitope polypeptide consists of an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

52. The polypeptide composition of claim 51, wherein the T-cell epitope polypeptide is linked to the N-terminus of the heterologous polypeptide.

53. The polypeptide composition of claim 51, wherein the T-cell epitope polypeptide is linked to the C-terminus of the heterologous polypeptide.

54. The polypeptide composition of claim 51, wherein the heterologous polypeptide comprises a biologically active molecule and wherein the biologically active molecule is selected from the group consisting of an immunogenic molecule, a T-cell epitope, a viral protein, and a bacterial protein.

55. The chimeric polypeptide composition of claim 51, wherein the heterologous polypeptide is operatively linked to the T-cell epitope polypeptide.

56. A method of inducing regulatory T-cells to suppress immune response in a subject comprising administrating to the subject a therapeutically effective amount of a polypeptide composition, wherein the polypeptide composition comprises one or more T-cell epitope polypeptides linked to a heterologous polypeptide, wherein the T-cell epitope polypeptide comprises an amino acid sequence selected from the group consisting of SEQ ID NOS: 1-73, and/or fragments and variants thereof, and optionally 1 to 12 additional amino acids distributed in any ratio on the N terminus and/or C-terminus of the polypeptide of SEQ ID NOS. 1-73.

57. The method of claim 56, wherein the T-cell epitope polypeptide is fused to the N-terminus of the heterologous polypeptide.

58. The method of claim 56, wherein the T-cell epitope polypeptide is fused to the C-terminus of the heterologous polypeptide.

59. The method of claim 56, wherein the heterologous polypeptide comprises a biologically active molecule and wherein the biologically active molecule is selected from the group consisting of an immunogenic molecule, a T-cell epitope, a viral protein, and a bacterial protein.

60. The method of claim 56, wherein the polypeptide composition further comprises an effective amount of one or more antigens and/or allergens.

61. The method of claim 56, wherein the immune suppressive effect is mediated by natural regulatory T-cells.

62. The method of claim 56, wherein the immune suppressive effect is mediated by adaptive regulatory T-cells.

63. The method of claim 56, wherein the T-cell epitope composition suppresses an effector T-cell response.

64. The method of claim 56, wherein the T-cell epitope composition suppresses a helper T-cell response.

68. The method of claim 56, wherein the T-cell epitope composition suppresses a B-cell response.

69. The method of claim 56, wherein the T-cell epitope composition suppresses a cytokine secretion of effector T-cells.

70. A method for reducing the immunogenicity of a human GAG/LYAG molecule or GAA replacement protein or supplement, comprising insertion of one or more regulatory T cell epitopes into the GAG/LYAG molecule or GAG replacement protein or supplement, wherein the one or more regulatory T cell epitopes are selected from the group consisting of: SEQ ID NOS: 1-73.

74. The method according to any one or claims 51 or 52, wherein the inserted regulatory T cell epitopes is not located in its natural position within the human GAG/LYAG molecule or GAG replacement protein or supplement or wherein the human GAG/LYAG molecule or GAG replacement protein or supplement thereof is missing such a Tregitope prior to said step of insertion.

75. A Tregitope-blood component conjugate comprising: a blood component linked to a modified polypeptide, said modified polypeptide having a reactive moiety attached thereto and said modified polypeptide comprising one or more regulatory T cell epitope, wherein said one or more regulatory T cell epitopes consists of one or more amino acid sequences selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

76. The Tregitope-blood component conjugate of claim 75, wherein said one or more regulatory T cell epitopes consists essentially of one or more amino acid sequences selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

77. The Tregitope-blood component conjugate of claim 75, wherein said one or more regulatory T cell epitopes comprises of one or more amino acid sequences selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

78. The Tregitope-blood component conjugate of any one of claims 75-77, wherein said blood component is albumin.

79. The Tregitope-blood component conjugate of any one of claims 75-78, wherein said blood component is human serum albumin.

80. The Tregitope-blood component conjugate of any one of claims 75-79, wherein the reactive moiety is attached to the amino terminal amino acid of the modified polypeptide.

81. The Tregitope-blood component conjugate of any one of claims 75-80, wherein the reactive moiety is attached to the carboxy terminal amino acid of the modified polypeptide.

82. The Tregitope-blood component conjugate of any one of claims 75-81, wherein the reactive moiety is attached to an amino acid positioned between the amino terminal amino acid and the carboxy terminal amino acid of the modified polypeptide.

83. The Tregitope-blood component conjugate of any one of claims 75-82, wherein the reactive moiety is a succinimidyl or maleimido group.

84. The Tregitope-blood component conjugate of any one of claims 75-83, wherein the reactive moiety is a 3-maleimidopropionic acid moiety.

85. The Tregitope-blood component conjugate of any one of claims 75-84, wherein the conjugation between the blood component and the modified polypeptide is a maleimide linkage.

86. A modified polypeptide, said modified polypeptide having a reactive moiety attached thereto and said modified polypeptide comprising one or more regulatory T cell epitopes consisting of one or more amino acid sequences selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

87. The modified polypeptide of claim 86, wherein said one or more regulatory T cell epitopes consists essentially of one or more amino acid sequences selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

88. The modified polypeptide of claim 87, wherein said one or more regulatory T cell epitopes comprises of one or more amino acid sequences selected from the group consisting of SEQ ID NOS: 1-14 and 74-116, and/or fragments and variants thereof, and optionally 1 to 12 additional amino acids distributed in any ratio on the N terminus and/or C-terminus of the polypeptide of SEQ ID NOS. 1-14 and 74-116.

89. The modified polypeptide of any one of claims 87-88, wherein the reactive moiety is attached to the amino terminal amino acid of the modified polypeptide.

90. The modified polypeptide of any one of claims 87-89, wherein the reactive moiety is attached to the carboxy terminal amino acid of the modified polypeptide.

91. The modified polypeptide of any one of claims 87-90, wherein the reactive moiety is attached to an amino acid positioned between the amino terminal amino acid and the carboxy terminal amino acid of the modified polypeptide.

92. The modified polypeptide of any one of claims 87-91, wherein the reactive moiety is a succinimidyl or maleimido group.

93. The modified polypeptide of any one of claims 87-92, wherein the reactive moiety is a 3-maleimidoproprionic acid moiety.
